(19) United States
(12) Patent Application Publication
(10) Pub. No.: US 2025/0264483 A1
(43) Pub. Date: Aug. 21, 2025

(54) IDENTIFICATION OF PROTHROMBOTIC CONDITIONS

(71) Applicant: Sydney Local Health District, Camperdown NSW (AU)

(72) Inventors: Christine Lee, Concord, New South Wales (AU); Vivien Chen, Concord, New South Wales (AU)

(21) Appl. No.: 18/033,107

(22) PCT Filed: Oct. 22, 2021
(86) PCT No.: PCT/AU2021/051233

§ 371 Date: Apr. 21, 2023

(30) Foreign Application Priority Data
Oct. 22, 2020 (AU) 2020903828
Jun. 30, 2021 (AU) 2021901983

(51) Int. Cl. G01N 33/86; G01N 15/14; G01N 33/50; G01N 33/68

(52) U.S. Cl. CPC G01N 33/86; G01N 15/14; G01N 33/5091; G01N 33/6893; G01N 2500/10; G01N 2800/226; G01N 2800/24

(57) ABSTRACT

Disclosed herein is a method of diagnosing an immune-mediated or inflammatory prothrombotic condition in a subject, the method comprising: a. Combining a sample of plasma obtained from the subject with a sample of whole blood obtained from a healthy donor to obtain a combined sample, b. Contacting the combined sample with at least one agonist to obtain a test sample, c. Determining the proportion of procoagulant platelets in the test sample and in at least one control sample, said determining comprising: i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and d. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition.

### CROSS-REFERENCE TO RELATED APPLICATIONS

The present application is a U.S. national-stage application of PCT/AU2021/051233, filed Oct. 21, 2021, which claims priority to Australian provisional application No's. 2020903828 and 2021901983, the entire disclosures of which are incorporated herein by cross-reference.

### FIELD

The present invention relates to methods of identifying whether a subject is suffering from, or at risk of suffering from, a prothrombotic condition.

### BACKGROUND

Hemostasis is the physiological response to vascular injury to control blood loss. Vascular injury initiates rapid activation and aggregation of platelets in the process of primary hemostasis, resulting in an unstable platelet plug. Secondary hemostasis involves activation of the plasma borne coagulation factors and is required to form a fibrin meshwork to stabilise the platelet plug. When this process occurs in excess, or at the wrong site, thrombosis occurs. Thrombosis is the pathological process of vascular occlusion by excess blood clot. It can occur in the arterial or venous systems, leading to clinical outcomes including myocardial infarction, stroke, deep venous thrombosis and as such, thrombosis remains the most common cause of death in industrialised nations.

Activated platelets have a dual role in hemostasis and thrombosis. They aggregate to form the platelet plug and also provide the surface for the assembly of the coagulation factors. A subset of activated platelets, which have been termed ‘procoagulant platelets’, promote coagulation by providing a procoagulant surface for the assembly and propagation of coagulation factors leading to the generation of thrombin. Generated through the cyclophilin-D dependent necrosis pathway, they contribute to thrombosis rather than platelet clearance. There is evidence that disturbances in this subset of platelets may be correlated to clinical outcomes in certain conditions. Excess procoagulant activity can tip the balance from physiological hemostasis to pathological thrombosis which means that platelet procoagulant activity is a profoundly important concept. The inventors have previously developed a flow cytometry assay which is able to determine the proportion of procoagulant platelets in a whole blood sample on the basis of co-labelling with GSAO (4-(N—(S-glutathionylacetyl)amino)phenylarsonous acid) and P-selectin (described in PCT/AU2015/000638).

The generation of procoagulant platelets, as identified by the inventors' GSAO-based assay, has not previously been studied in immune-mediated or inflammatory prothrombotic conditions, nor used diagnostically.

In certain conditions, thrombotic events may occur due to the action of inflammatory or immune mediators on platelets. Examples of such conditions include heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers.

There is a need for improved methods of identifying such conditions and/or of identifying whether a subject suffering from such a condition is at risk of a thrombotic event.

### Heparin-Induced Thrombocytopenia

One example of an immune-mediated or inflammatory prothrombotic condition is heparin-induced thrombocytopenia (HIT). HIT is a prothrombotic adverse drug reaction to the commonly used anticoagulant heparin, or its derivatives. HIT is associated with high rates of morbidity and mortality, with thrombotic complications affecting over 50% of untreated patients. HIT occurs in up to 3% of patients receiving unfractionated heparin, and 0.2% of patients on low-molecular-weight heparin.

Key to the pathogenesis of HIT is the formation of platelet-activating immunoglobulin G (IgG) antibodies recognising complexes of the platelet alpha granule protein, platelet factor 4 (PF4), and heparin. These HIT antibodies, together with PF4-heparin complexes, form immune complexes, which are able to bind and cross-link the low-affinity IgG receptor, FcγRIIa, on platelets, monocytes and neutrophils, causing platelet activation and aggregation through the immunoreceptor tyrosine-based activation motif (ITAM). Thrombocytopenia and thrombosis occur as a result, augmented by increased platelet clearance, the release of platelet-derived microparticles, and thrombin produced on the surface of activated monocytes.

Both the diagnosis and treatment of HIT remain clinical challenges. Using existing techniques, a confirmed HIT diagnosis requires laboratory findings in addition to clinical features, the gold standard functional laboratory test being the serotonin release assay (SRA). This platelet functional test measures the release of radiolabeled-serotonin from washed platelets at both therapeutic (0.1-0.3 U/mL) and high (100 U/mL) doses of heparin, with a positive test characterised by greater than 20% release at therapeutic dose that is inhibited at high heparin concentration. The basis of the high dose heparin confirmation step relates to the molar stoichiometry of PF4 and heparin, under which if either is in excess, complexes do not form and FcγRIIa is not activated. The limitations associated with the SRA are well documented—including requirements for washed pedigree donor platelets and radioactive-labeled serotonin. These features limit its use to the rare reference laboratory, for example a single laboratory in Australia. These restrictions mean that a definitive diagnosis of HIT is often delayed, and treatment decisions must often be made prior to confirmation of either diagnosis or exclusion of HIT.

There is therefore specifically a need for an improved method for identification of HIT, for example a method which may distinguish HIT from thrombocytopenia not induced by the administration of heparin.

In a previous study, Tutwiler and colleagues (Blood, 2016, 127(4):464-472) used Annexin V as a marker of procoagulant “coated” platelets in the KKO (a murine monoclonal antibody that mimics heparin-induced thrombocytopenia antibodies) and PF4 HIT phenotype model. However, the procoagulant platelet population identified by the inventors' GSAO-based assay is different to the “coated” platelet subpopulation identified by Annexin V labelling. Furthermore, the Tutwiler study did not investigate actual HIT patient samples, but a HIT phenotype model. As a phenotype model, the Tutwiler study is limited to observing the effects of KKO only, which is a single monoclonal antibody. Studies utilising actual HIT patient samples would be affected by the presence of a variety of different plasma factors.

### Vaccine-Induced Immune Thrombotic Thrombocytopenia (VITT)

Another example of an immune-mediated or inflammatory prothrombotic condition is vaccine-induced immune thrombotic thrombocytopenia (VITT). A syndrome of vaccine-induced immune thrombotic thrombocytopenia (VITT) has been described in a proportion of patients vaccinated against severe acute respiratory distress syndrome coronavirus 2 (SARS-CoV-2). VITT is a severe prothrombotic complication of adenoviral vaccines including ChAdOx1 nCoV-19 (AstraZeneca/Vaxzevria) vaccine and Johnson & Johnson/Janssen JNJ-78436735 COVID-19 vaccine. The putative mechanism is analogous to heparin-induced thrombocytopenia (HIT) which involves formation of pathological antibodies against platelet factor 4 (PF4) that activate platelets via the low-affinity IgG receptor FcγRIIa to drive thrombosis and the associated thrombocytopenia. Functional assays are important in the VITT diagnostic pathway as not all detectable PF4 antibodies are pathogenic. VITT-related thrombosis can be severe and on occasion, fatal. The mortality rate is significantly better if patients are recognised early, and alternative therapy is commenced including infusion of intravenous immunoglobulin and commencement of non-heparin anticoagulants. Hence, accurate and timely diagnosis is critical.

Mass vaccination is imperative in the global battle against COVID-19 but rare thrombotic events associated with SARS-CoV-2 vaccination raise concerns on the safety of adenoviral vector vaccines. Early and accurate diagnosis of the potentially fatal VITT syndrome has far-reaching implications both from a public health perspective and for clinical management as early intervention significantly improves patient outcomes. There is a need for an improved method for identification of VITT, for example a method which may distinguish VITT from thrombocytopenia not induced by the administration of a vaccine.

Detection of procoagulant platelets using a combination of P-selectin (platelet activation marker) and Annexin V (phosphatidylserine marker) in presence of PF4 has been proposed as a diagnostic assay for VITT (Althaus K, Möller P, Uzun G, et al. Antibody-mediated procoagulant platelets in SARS-CoV-2-vaccination associated immune thrombotic thrombocytopenia. Haematologica. 2021; 106(8):2170-2179). However, this assay has the disadvantage of requiring washed platelets. It is also noted that the combined Annexin V/P-selectin markers do not identify functionally procoagulant platelets, i.e. do not identify the same population of platelets as the assay of the present invention. Another diagnostic assay for VITT is PF4-induced flow cytometry-based platelet activation by Handtke and colleagues (Handtke S, Wolff M, Zaninetti C, et al. A flow cytometric assay to detect platelet-activating antibodies in VITT after ChAdOx1 nCov-19 vaccination. Blood. 2021; 137(26):3656-3659), which is performed on hirudinated donor whole blood with addition of exogenous PF4 with P-selectin MFI as an output. It is noted that this assay does not measure procoagulant platelets. Further, the use of exogenous PF4, which features in both of the above assays, has a number of disadvantages such as the variability introduced by the varying oligomerization state of PF4 which is influenced by its method of preparation, and its high cost.

### Anti-Phospholipid Syndrome

Another example of an immune-mediated or inflammatory prothrombotic condition is anti-phospholipid syndrome (APS). APS is an autoimmune disease in which the body generates so-called ‘anti-phospholipid antibodies’, which includes antibodies against cardiolipin, beta-2 glycoprotein 1, and the lupus anticoagulant. These substances activate platelets and white cells, leading to activation of the coagulation system and may lead to thrombosis.

Subjects suffering from APS may be generally symptom free, but periodically, or in certain circumstances, be at increased risk of suffering a thrombotic event. When a subject suffering from anti-phospholipid syndrome is at increased risk of suffering a thrombotic event, they require treatment for APS, such as anticoagulation treatment, anti-platelet agents, intravenous immunoglobulin, or hydroxychloroquine, and on occasion, immunosuppression (such as steroids and chemotherapy such as cyclophosphamide) or plasma exchange, to be administered or escalated. It is important to be able to identify when such treatment is required, but also when such treatment is not required, as the treatment may result in dangerous side effects such as increased bleeding and infection rates.

### SUMMARY OF THE INVENTION

In a first aspect of the invention, there is provided a method of diagnosing an immune-mediated or inflammatory prothrombotic condition in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition.

The following options may be used in conjunction with the first aspect of the invention, either individually or in any combination.

The assay control sample may be obtained by contacting said test sample or said at least one control sample with GSCA and a control for the alpha granule detection agent. The alpha granule detection agent may be an antibody and the control for the alpha granule detection agent is an antibody isotype control.

The increased proportion of procoagulant platelets in the test sample compared to the at least one control sample which indicates that the subject is suffering from the immune-mediated or inflammatory prothrombotic condition may be an increase of at least about 1.1-fold.

Determining the uptake of GSAO and surface expression of alpha granule protein of platelets in step c.i. may be carried out by flow cytometry.

The alpha granule detection agent may be selected from the group consisting of an anti-P-selectin antibody, and an anti-TLT-1 antibody. The alpha granule detection agent may be an anti-P-selectin antibody.

The healthy donor may be a FcγRIIa high responder. The test sample may contain less than 100 μL of whole blood obtained from the healthy donor.

The test sample may not contain exogeneous PF4.

The at least one agonist may be selected from the group consisting of CRP-xL, SFLLRN, ADP, thrombin, and combinations thereof.

The immune-mediated or inflammatory prothrombotic condition may be selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers.

Where the immune-mediated or inflammatory prothrombotic condition is selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers, the at least one control sample may comprise a healthy control sample, which comprises healthy donor whole blood contacted with the agonist, and does not comprise subject plasma. In this case the increased proportion of procoagulant platelets in the test sample compared to the healthy control sample which indicates that the subject is suffering from the prothrombotic condition is an increase of at least about 1.1-fold.

### Diagnosis of Heparin-Induced Thrombocytopenia (HIT)

The immune-mediated or inflammatory prothrombotic condition may be heparin-induced thrombocytopenia (HIT). In this case step b. may further comprise contacting the test sample with therapeutic concentration heparin and the at least one control sample may comprise a no heparin control sample and a high heparin control sample, wherein the no heparin control sample comprises the combined sample, the agonist, and no heparin, and wherein the high heparin control sample comprises the combined sample, the agonist, and high concentration heparin. The therapeutic concentration of heparin may be between about 0.1 and about 0.7 U/mL and the high concentration of heparin may be about 50 to 200 U/mL.

The at least one agonist may be selected from the group consisting of CRP-xL, SFLLRN, ADP, and combinations thereof.

The at least one agonist may be SFLLRN. In this case the proportion of procoagulant platelets in the test sample contacted with therapeutic concentration heparin compared to the no heparin control sample which indicates that the subject is suffering from HIT is an increase of between about 2 and 10-fold, and the proportion of procoagulant platelets in the test sample contacted with therapeutic concentration heparin compared to the high heparin control sample which indicates that the subject is suffering from HIT is an increase of between about 2 and 20-fold.

The method of the first aspect of the invention may be capable of differentiating a subject suffering from HIT from a subject suffering from acute thrombocytopenia which is not heparin-induced.

### Diagnosis of Vaccine-Induced Immune Thrombotic Thrombocytopenia (VITT)

The immune-mediated or inflammatory prothrombotic condition may be vaccine-induced immune thrombotic thrombocytopenia (VITT).

The subject may have been administered a vaccine. The vaccine may be a recombinant adenoviral vector vaccine, particularly a recombinant adenoviral vector encoding the spike protein antigen of SARS-CoV-2. The vaccine is selected from the AstraZeneca AZD1222 (Vaxzevria) vaccine, the Johnson & Johnson/Janssen JNJ-78436735 vaccine, the CanSino Biologics AD5-nCOV vaccine, and the Gamaleya Research Institute of Epidemiology and Microbiology Gam-COVID-Vac vaccine.

The at least one agonist may be selected from the group consisting of SFLLRN and thrombin.

The at least one agonist may be SFLLRN. In this case step b. may further comprise contacting the test sample with therapeutic concentration heparin to obtain a therapeutic heparin sample, and further comprise contacting the test sample with high concentration heparin to obtain a high heparin sample, and step d. may further comprise determining the proportion of procoagulant platelets in the therapeutic heparin sample and the high heparin sample. The at least one control sample may be a healthy control sample, comprising healthy donor whole blood contacted with the at least one agonist, and does not comprise subject plasma. The therapeutic concentration of heparin may be between about 0.1 and about 0.7 U/m, and the high concentration of heparin may be about 50 to 200 U/mL.

In this case, compared to the healthy control sample:
(i) an increase in the proportion of procoagulant platelets in the test sample of greater than 2-fold, a change in the proportion of procoagulant platelets in the therapeutic heparin sample of up to 1.5-fold, and a change in the proportion of procoagulant platelets in the high heparin sample of up to 1.5-fold, indicates that the subject is suffering from classical VITT;
(ii) an increase in the proportion of procoagulant platelets in the test sample of greater than 1-fold, an increase in the proportion of procoagulant platelets in the therapeutic heparin sample of greater than 2-fold, and a change in the proportion of procoagulant platelets in the high heparin sample of up to 1.5-fold, indicates that the subject is suffering from heparin-enhancing VITT; and
(iii) a change in the proportion of procoagulant platelets in the test sample of up to 1.5-fold, a change in the proportion of procoagulant platelets in the therapeutic heparin sample of up to 1.5-fold, and a change in the proportion of procoagulant platelets in the high heparin sample of up to 1.5-fold, indicates that the subject is not suffering from VITT,
wherein the healthy control sample comprises healthy donor whole blood contacted with the at least one agonist, and does not comprise subject plasma.

The test sample may be further contacted with the monoclonal antibody IV.3. In this case:
(iv) a decrease in the proportion of procoagulant platelets in the test sample further contacted with the monoclonal antibody IV.3 to 0.7-fold or greater indicates that the subject is suffering from classical VITT or heparin-enhancing VITT.

Alternatively, the at least one agonist may be thrombin. In this case, the at least one control sample may be a healthy control sample, comprising healthy donor whole blood contacted with the at least one agonist, and does not comprise subject plasma and the increased proportion of procoagulant platelets in the test sample compared to the healthy control sample which indicates that the subject is suffering from VITT is an increase of at least about 2-fold.

The method of the first aspect of the invention may be capable of differentiating a subject suffering from VITT from a subject suffering from acute thrombocytopenia which is not vaccine-induced.

### Diagnosis of Antiphospholipid Syndrome

The prothrombotic condition may be antiphospholipid syndrome.

In this case, the at least one control sample may comprise a healthy control sample, which comprises healthy donor whole blood contacted with the agonist, and does not comprise subject plasma.

The agonist may be thrombin and the increased proportion of procoagulant platelets in the test sample compared to the healthy control sample which indicates that the subject is suffering from the prothrombotic condition may be an increase of at least about 1.1-fold.

In a second aspect of the invention there is provided a method of treatment of an immune-mediated or inflammatory prothrombotic condition in a subject, the method comprising:
a. Determining if the subject is suffering from the immune-mediated or inflammatory prothrombotic condition, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition,
b. Administering a treatment for the immune-mediated or inflammatory prothrombotic condition to the subject, only if the subject is determined to be suffering from the immune-mediated or inflammatory prothrombotic condition.

In a third aspect of the invention there is provided a method of treatment of an immune-mediated or inflammatory prothrombotic condition in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for the immune-mediated or inflammatory prothrombotic condition, only if the subject is at risk of experiencing a thrombotic event.

In a fourth aspect of the invention, there is provided a method of selecting a therapy for treatment of an immune-mediated or inflammatory prothrombotic condition in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of the immune-mediated or inflammatory prothrombotic condition if the proportion of procoagulant platelets in the therapy test sample is lower than the proportion of procoagulant platelets in the test sample.

In a fifth aspect of the invention, there is provided a method of determining the duration of treatment of an immune-mediated or inflammatory prothrombotic condition in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of the immune-mediated or inflammatory prothrombotic condition only if the subject exhibits an increased proportion of procoagulant platelets, or
c. Ceasing administration of treatment of the immune-mediated or inflammatory prothrombotic condition if the subject does not exhibit an increased proportion of procoagulant platelets.

In a sixth aspect of the invention, there is provided a method of reducing side effects associated with a treatment of an immune-mediated or inflammatory prothrombotic condition in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for the immune-mediated or inflammatory prothrombotic condition to the subject, only if the subject is at risk of experiencing a thrombotic event, or
c. Not administering or not escalating the treatment for the immune-mediated or inflammatory prothrombotic condition to the subject, if the subject is not at risk of experiencing a thrombotic event.

The following options may be used in conjunction with the second to sixth aspects of the invention, either individually or in any combination.

The assay control sample may be obtained by contacting said test sample or said at least one control sample with GSCA and a control for the alpha granule detection agent. The alpha granule detection agent may be an antibody and the control for the alpha granule detection agent is an antibody isotype control.

The increased proportion of procoagulant platelets in the test sample compared to the at least one control sample which indicates that the subject is suffering from the immune-mediated or inflammatory prothrombotic condition may be an increase of at least about 1.1-fold.

Determining the uptake of GSAO and surface expression of alpha granule protein of platelets in step c.i. may be carried out by flow cytometry.

The alpha granule detection agent may be selected from the group consisting of an anti-P-selectin antibody, and an anti-TLT-1 antibody. The alpha granule detection agent may be an anti-P-selectin antibody.

The healthy donor may be a FcγRIIa high responder. The test sample may contain less than 100 μL of whole blood obtained from the healthy donor.

The test sample may not contain exogeneous PF4.

The at least one agonist may be selected from the group consisting of CRP-xL, SFLLRN, ADP, thrombin, and combinations thereof.

The immune-mediated or inflammatory prothrombotic condition may be selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers.

Where the immune-mediated or inflammatory prothrombotic condition is selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers, the at least one control sample may comprise a healthy control sample, which comprises healthy donor whole blood contacted with the agonist, and does not comprise subject plasma. In this case the increased proportion of procoagulant platelets in the test sample compared to the healthy control sample which indicates that the subject is suffering from the prothrombotic condition is an increase of at least about 1.1-fold.

Treatment Etc. Of Heparin-Induced Thrombocytopenia (HIT)

The immune-mediated or inflammatory prothrombotic condition may be heparin-induced thrombocytopenia (HIT). In this case step b. may further comprise contacting the test sample with therapeutic concentration heparin and the at least one control sample may comprise a no heparin control sample and a high heparin control sample, wherein the no heparin control sample comprises the combined sample, the agonist, and no heparin, and wherein the high heparin control sample comprises the combined sample, the agonist, and high concentration heparin. The therapeutic concentration of heparin may be between about 0.1 and about 0.7 U/mL and the high concentration of heparin may be about 50 to 200 U/mL.

The at least one agonist may be selected from the group consisting of CRP-xL, SFLLRN, ADP, and combinations thereof.

The at least one agonist may be SFLLRN. In this case the proportion of procoagulant platelets in the test sample contacted with therapeutic concentration heparin compared to the no heparin control sample which indicates that the subject is suffering from HIT is an increase of between about 2 and 10-fold, and the proportion of procoagulant platelets in the test sample contacted with therapeutic concentration heparin compared to the high heparin control sample which indicates that the subject is suffering from HIT is an increase of between about 2 and 20-fold.

In the method of the second or third aspects of the invention, administering a treatment for HIT may comprise ceasing the administration of heparin and commencing administration of a non-heparin anticoagulant.

The method of the second or third aspects of the invention may further comprise:
c. continuing the administration of heparin, only if the subject is determined to not be suffering from HIT.
   Treatment Etc. Of Vaccine-Induced Immune Thrombotic Thrombocytopenia (VITT)

The immune-mediated or inflammatory prothrombotic condition may be vaccine-induced immune thrombotic thrombocytopenia (VITT).

The subject may have been administered a vaccine. The vaccine may be a recombinant adenoviral vector vaccine, particularly a recombinant adenoviral vector encoding the spike protein antigen of SARS-CoV-2. The vaccine is selected from the AstraZeneca AZD1222 (Vaxzevria) vaccine, the Johnson & Johnson/Janssen JNJ-78436735 vaccine, the CanSino Biologics AD5-nCOV vaccine, and the Gamaleya Research Institute of Epidemiology and Microbiology Gam-COVID-Vac vaccine.

The at least one agonist may be selected from the group consisting of SFLLRN and thrombin.

The at least one agonist may be SFLLRN. In this case step b. may further comprise contacting the test sample with therapeutic concentration heparin to obtain a therapeutic heparin sample, and further comprise contacting the test sample with high concentration heparin to obtain a high heparin sample, and step d. may further comprise determining the proportion of procoagulant platelets in the therapeutic heparin sample and the high heparin sample. The at least one control sample may be a healthy control sample, comprising healthy donor whole blood contacted with the at least one agonist, and does not comprise subject plasma. The therapeutic concentration of heparin may be between about 0.1 and about 0.7 U/m, and the high concentration of heparin may be about 50 to 200 U/mL.

In this case, compared to the healthy control sample:
(i) an increase in the proportion of procoagulant platelets in the test sample of greater than 2-fold, a change in the proportion of procoagulant platelets in the therapeutic heparin sample of up to 1.5-fold, and a change in the proportion of procoagulant platelets in the high heparin sample of up to 1.5-fold, indicates that the subject is suffering from classical VITT;
(ii) an increase in the proportion of procoagulant platelets in the test sample of greater than 1-fold, an increase in the proportion of procoagulant platelets in the therapeutic heparin sample of greater than 2-fold, and a change in the proportion of procoagulant platelets in the high heparin sample of up to 1.5-fold, indicates that the subject is suffering from heparin-enhancing VITT; and
(iii) a change in the proportion of procoagulant platelets in the test sample of up to 1.5-fold, a change in the proportion of procoagulant platelets in the therapeutic heparin sample of up to 1.5-fold, and a change in the proportion of procoagulant platelets in the high heparin sample of up to 1.5-fold, indicates that the subject is not suffering from VITT,
wherein the healthy control sample comprises healthy donor whole blood contacted with the at least one agonist, and does not comprise subject plasma.

The test sample may be further contacted with the monoclonal antibody IV.3. In this case:
(iv) a decrease in the proportion of procoagulant platelets in the test sample further contacted with the monoclonal antibody IV.3 to 0.7-fold or greater indicates that the subject is suffering from classical VITT or heparin-enhancing VITT.

Alternatively, the at least one agonist may be thrombin. In this case, the at least one control sample may be a healthy control sample, comprising healthy donor whole blood contacted with the at least one agonist, and does not comprise subject plasma and the increased proportion of procoagulant platelets in the test sample compared to the healthy control sample which indicates that the subject is suffering from VITT is an increase of at least about 2-fold.

In the method of the second or third aspects of the invention, administering a treatment for VITT may comprise:
(i) ceasing the administration of heparin if the subject has been administered heparin, and commencing administration of a non-heparin anticoagulant; and/or
(ii) commencement of immunoglobulin therapy (IVIg); and/or
(iii) commencement of immunosuppressive therapy; and/or
(iv) commencement of plasma exchange.

The method of the second or third aspects of the invention may further comprise:
c. administration of heparin, only if the subject is determined to not be suffering from VITT.

In the method of the fourth aspect of the invention, where the prothrombotic condition is VITT, the therapy may be selected from the group consisting of plasma exchange, non-heparin anticoagulants and immunoglobulin (IVIg). The non-heparin coagulant may be fondaparinux.

Treatment Etc. Of Antiphospholipid Syndrome

The prothrombotic condition may be antiphospholipid syndrome.

In this case, the at least one control sample may comprise a healthy control sample, which comprises healthy donor whole blood contacted with the agonist, and does not comprise subject plasma.

The agonist may be thrombin and the increased proportion of procoagulant platelets in the test sample compared to the healthy control sample which indicates that the subject is suffering from the prothrombotic condition may be an increase of at least about 1.1-fold.

The method of the first to sixth aspects of the invention may be carried out in vitro.

### BRIEF DESCRIPTION OF DRAWINGS

FIG. 1. Plasma samples from SRA-positive (HIT+) patients sensitized healthy donor platelets to form procoagulant platelets when treated with strong platelet agonists. Whole blood from healthy donors was treated with plasma samples from either HIT immunoassay-negative (HIT−) or HIT+ patients, and a combination of thrombin (1 U/mL) and CRP-xL (2 μg/mL) for 10 min, then stained for flow cytometry analysis of procoagulant platelets. FIG. 1A: Flow cytometry contour plots of platelets from a healthy donor treated with thrombin and CRP-xL alone (no plasma), or in the presence of HIT− or HIT+ plasma. Procoagulant platelets were defined as GSAO+/CD62P+ platelets (outlined in red). Percentage of all platelet events is shown in each quadrant. FIG. 1B: The proportion of procoagulant platelets from healthy donors (n=9-11) increased significantly upon exposure to HIT− (n=5) or HIT+ (n=4) plasma when treated with thrombin and CRP-xL, compared to no plasma (nil). Mixed-effects analysis was performed with Tukey's correction for multiple comparisons. Error bars indicate mean±SEM. *P<0.05, ***P<0.001, ****P<0.0001. CRP-xL, crosslinked collagen-related peptide; GSAO, [4-(N—(S-glutathionylacetyl)amino)phenylarsonous acid]; SRA, serotonin release assay.

FIG. 2. Plasma samples from HIT patients do not increase the proportion of procoagulant platelets from healthy donors when treated with either platelet agonists or heparin, alone. (A) The proportion of procoagulant platelets in whole blood samples from healthy donors (n=6-13) did not change significantly upon exposure to HIT− (n=5) or HIT+ (n=4) plasma under resting conditions or treatment with platelet agonists (SFLLRN 5 μM, ADP 20 μM, CRP-xL 2 μg/mL). Mixed-effects analysis was performed with Tukey's correction for multiple comparisons. (B) In the absence of stimulation with platelet agonist, the addition of low-dose (0.5 U/mL) or high-dose (100 U/mL) heparin to platelets from healthy donors (n=6-7) exposed to HIT− (n=3) or HIT+ (n=4) plasma did not alter the basal proportion of procoagulant platelets observed in the absence of patient plasma. One-way ANOVA was performed with Dunnett's correction for multiple comparisons to basal level of procoagulant platelets. Error bars indicate mean±SEM. ns, not significant. ADP, adenosine diphosphate; CRP-xL, crosslinked collagen-related peptide; Hep, heparin; SFLLRN, thrombin receptor-activating peptide.

FIG. 3. Plasma samples from SRA-positive (HIT+) but not HIT immunoassay-negative (HIT−) patients increased the procoagulant platelet response in donor platelets treated with platelet agonists in a heparin-dependent manner and requires active FcγRIIa. Whole blood from healthy donors (n=6-10) were treated with plasma samples from either HIT− (n=5, A, C, E) or HIT+ (n=3-4, B, D, F) patients, in the absence of heparin, or addition of low-dose (0.5 U/mL) or high-dose (100 U/mL) heparin and platelet agonists: (A-B) thrombin receptor-activating peptide, SFLLRN (5 μM) or (C-D) adenosine diphosphate, ADP (20 μM) or (E-F) crosslinked collagen-related peptide, CRP-xL (2 μg/mL) for 10 min before staining for flow cytometry analysis of procoagulant platelets. Repeated measures ANOVA was performed with Tukey's correction for multiple comparisons. (G) Donor platelets (n=6-7) in whole blood were pre-treated with the FcγRIIa function-blocking monoclonal antibody, IV.3 (10 μg/mL) for 15 min before exposure to SRA-positive HIT plasma (n=4) and platelet agonists. In the presence of low-dose heparin (0.5 U/mL) and platelet agonists: CRP-xL (2 μg/mL), SFLLRN (5 μM) or ADP (20 μM), pre-treatment with IV.3 abrogated the procoagulant platelet proportions compared to vehicle control. Paired t-test was performed comparing vehicle to IV.3 treatment for each agonist. Procoagulant platelet percentages were defined by the proportion of GSAO+/CD62P+ platelet events. Error bars indicate mean±SEM. *P<0.05, **P<0.01, ns, not significant. Hep, heparin; SRA, serotonin release assay.

FIG. 4. Procoagulant platelet formation in the presence of platelet agonists, low-dose heparin and patient plasma has potential to differentiate thrombocytopenia secondary to HIT from other etiologies. Receiver operating characteristic (ROC) curve analysis was performed to evaluate the diagnostic potential of procoagulant platelet formation in healthy donors (n=6-11) induced by plasma from patients suspected of HIT in the presence of low-dose heparin (0.5 U/mL) and receptor-specific platelet agonists: (A) thrombin receptor-activating peptide, SFLLRN (5 μM), (B) adenosine diphosphate, ADP (20 μM) or (C) crosslinked collagen-related peptide, CRP-xL (2 μg/mL). Plasma samples were collected from either HIT immunoassay-negative patients (HIT−) or SRA-confirmed HIT patients (HIT+). Error bars indicate mean SEM. AUC, area under the curve; SRA, serotonin release assay

FIG. 5. Validation of procoagulant platelet assay using plasma samples from 64 patients suspected of HIT. (A) Whole blood from healthy donors was treated with 5 μM SFLLRN and therapeutic-concentration (0.5 U/mL) or high-dose (100 U/mL) heparin in the presence of plasma from immunoassay-negative or clinically not HIT patients (true negative, black circles; n=41) or SRA-confirmed and clinically verified HIT patients (true positives, orange circles; n=18). Blue circles represent plasma samples that were immunoassay-positive but SRA-negative and clinically adjudicated as not HIT (n=3). The half-orange and half-blue circle represents a patient who was immunoassay-positive and clinically verified as HIT but SRA-negative (n=1) while the orange open circle represents a false negative (n=1) on the procoagulant platelet assay. A positive result is defined by a procoagulant platelet percentage of greater than 7.6% as determined in FIG. 4A. The assay was performed while blinded to the diagnosis of the patients. (B) Procoagulant platelet response of healthy donors to clinically adjudicated HIT+ plasma (n=21) in the presence of 5 μM SFLLRN and 0.5 U/mL heparin, and (C) anti-PF4/heparin antibody levels of 11 HIT+ plasma, measured on AcuStar HIT-IgG_{(PF4-H)} chemiluminescence immunoassay was compared between patients with and without thrombotic outcomes based on review of clinical notes while blinded to assay results. Unpaired t-test was performed. Error bars indicate mean±SEM. ns, not significant, *P<0.05. SFLLRN, thrombin receptor-activating peptide; Hep, heparin; SRA, serotonin release assay.

FIG. 6. Plasma samples from SRA-positive (HIT+) patients sensitized healthy donor platelets to form procoagulant platelets when treated with thrombin in a manner that is partially dependent on FcγRIIa. Whole blood from healthy donors were treated with plasma samples from either HIT immunoassay-negative (HIT−) or HIT+ patients, and thrombin alone or a combination of thrombin and CRP-xL for 10 min, then stained for flow cytometry analysis of procoagulant platelets. (A-C) The proportion of procoagulant platelets from healthy donors (n=27) exposed to HIT− (n=24) or HIT+ (n=8) plasma when treated with thrombin (1 U/mL) or with the addition of FcγRIIa function-blocking monoclonal antibody, IV.3 (10 μg/mL). (D-F) The proportion of procoagulant platelets from healthy donors (n=41) exposed to HIT− (n=36) or HIT+ (n=15) plasma when treated with thrombin (1 U/mL) and CRP-xL (2 μg/mL) or with the addition of IV.3 (10 μg/mL). (A and D) Mixed-effects analysis was performed with Tukey's correction for multiple comparisons. (B-C and E-F) Repeated measures ANOVA was performed with Tukey's correction for multiple comparisons. Error bars indicate mean±SEM. *P<0.05, **P<0.01, ***P<0.001, ****P<0.0001, ns, not significant. CRP-xL, crosslinked collagen-related peptide; SRA, serotonin release assay.

FIG. 7. Procoagulant platelet formation in the presence of T1CRP2 is increased in patients having a variety of immune-mediated or inflammatory prothrombotic conditions. Data points marked P1 represent a patient suffering from anti-phospholipid syndrome. Data points marked P2 represent a patient suffering from toxic epidermal necrolysis secondary to chemotherapy.

FIG. 8. Plasma samples from VITT-positive patients sensitised healthy donor platelets to form procoagulant platelets when treated with platelet agonists. Whole blood from healthy donors was treated with plasma samples from VITT positive, HIT-like VITT (heparin-enhancing VITT) or VITT negative patients and 5 μM SFLLRN for 10 min, then stained for flow cytometry analysis of procoagulant platelets. FIG. 8A: Flow cytometry contour plots of platelets from a healthy donor treated with 5 μM SFLLRN alone (no plasma), or in the presence of VITT positive, HIT-like VITT (heparin-enhancing VITT) or VITT negative plasma. Procoagulant platelets were defined as GSAO+/CD62P+ platelets (outlined in bold/red). Percentage of all platelet events is shown in each quadrant. FIG. 8B: The proportion of procoagulant platelets from healthy donors (n=20) upon exposure to VITT positive (n=24) or HIT-like VITT (heparin-enhancing VITT) (n=2) plasma when treated with SFLLRN, compared to no plasma or VITT negative plasma (n=27). FIG. 8C: The proportion of procoagulant platelets from healthy donors (n=19) upon exposure to VITT positive (n=24), HIT-like VITT (heparin-enhancing VITT) (n=2) or VITT negative (n=26) plasma when treated with 1 U/mL thrombin. Kruskal-Wallis test was performed with Dunn's correction for multiple comparisons. Error bars indicate mean±SD. *P<0.05, ****P<0.0001.ns, not significant.. SFLLRN, thrombin receptor-activating peptide.

FIG. 9. Plasma from VITT patients sensitizes healthy donor platelets to become procoagulant. (A) Schematic diagram and representative flow cytometry plots depicting platelet P-selectin expression (CD62P) and GSAO uptake on (i) unstimulated donor platelets and upon stimulation with (ii) 5 μM SFLLRN alone or (iii) a synergistic combination of SFLLRN and plasma containing VITT anti-PF4 antibodies or (iv) plasma from a patient with bacterial sepsis. Procoagulant platelets are defined as GSAO+/CD62P+ platelet events (red quadrant). (B) Confocal imaging of healthy donor platelet rich plasma exposed to 5 μM SFLLRN alone or SFLLRN and VITT plasma. Platelets are identified by CD41a antibody (cyan, top panel) while P-selectin (CD62P in yellow, middle panel) marks activated platelets. GSAO uptake is shown in yellow (bottom panel). Procoagulant platelets are characterized by ballooning morphology and GSAO uptake (bottom right panel). (C) Procoagulant platelet flow cytometry was performed using healthy donor whole blood treated with platelet agonist 5 μM SFLLRN (n=43) and incubated with plasma from healthy individuals (n=32), ChAdOx-1 nCoV-2 vaccinated patients with thrombocytopenia and thrombosis but without detectable anti-PF4 antibodies (VITT neg, n=20), vaccinated patients who were not thrombocytopenic with detectable anti-PF4 antibodies (ELISA false pos, n=4) or clinically confirmed SRA-positive VITT patients with thrombocytopenia, thrombosis and detectable anti-PF4 antibodies (VITT pos, n=23). Procoagulant platelet percentages were defined by the proportion of GSAO+/CD62P+ platelet events. Kruskal-Wallis test with Dunn's correction for multiple comparisons was performed. Error bars indicate mean±SD. *p<0.05, ****p<0.0001.

FIG. 10. Procoagulant platelet response induced by VITT plasma is suppressed by heparin, is antibody mediated and corresponded with clinical response to therapy. Healthy donor whole blood was treated with platelet agonist 5 μM SFLLRN and plasma from (A) VITT (n=23) or (B) HIT (n=8) patients in presence of low- (0.5 U/mL) or high-dose (100 U/mL) unfractionated heparin and assessed for procoagulant platelet formation by flow cytometry. Blue lines represent VITT patients that generated a heparin-enhancing procoagulant platelet response at low-dose heparin. Friedman test with Dunn's correction for multiple comparisons was performed. Donor blood was pre-treated with (C) an FcγRIIa-blocking antibody IV.3 (10 μg/mL) or (D) intravenous immunoglobulin (IVIg, 10 mg/mL) for 15 min prior to exposure to VITT plasma and 5 μM SFLLRN. Wilcoxon matched-pairs signed rank test was performed. (E) Procoagulant platelet response induced by VITT plasma (n=4) collected pre-intravenous immunoglobulin (IVIg) treatment was compared with the procoagulant response within 5 days post-IVIg therapy. The reduction in the procoagulant platelet response corresponded to the suppressive effect of exogenous IVIg (10 mg/mL) on the pre-IVIg sample. Results are presented as fold-change in procoagulant platelet proportion relative to no plasma control. Each line represents a unique patient. (F) Plasma samples from 6 VITT patients collected prior to initiation of fondaparinux treatment was tested in presence of fondaparinux (1.2 μg/mL), in vitro.. Procoagulant platelet response is normalized to no fondaparinux control. Four patients were subsequently found to be clinically responsive to fondaparinux while two patients were fondaparinux-resistant. Healthy donor whole blood was treated with platelet agonist 5 μM SFLLRN and VITT plasma in presence of (G) ChAdOx1 nCoV-19 vaccine (AZD1222, 1:2000 (v/v), n=23) or (H) recombinant SARS-CoV-2 spike protein (HexaPro, 20 μg/mL, n=10) before flow cytometric assessment for procoagulant platelet formation. Wilcoxon matched-pairs signed rank test was performed. *p<0.05, **p<0.01, ****p<0.0001, ns, not significant.

FIG. 11. Diagnostic potential of procoagulant platelet flow cytometry assay in identifying VITT plasma. (A) Receiver operating characteristic (ROC) curve analysis was performed to evaluate the diagnostic potential of fold-increase compared to no plasma baseline in procoagulant platelet formation in healthy donors induced by plasma from confirmed VITT patients (n=23) who tested positive on both ELISA and SRA, and VITT-negative patients (n=24) who tested negative on both ELISA and SRA and ELISA false positive patients who are non-thrombocytopenic and SRA-negative. (B) ROC analysis of the fold-change in procoagulant platelet proportion in presence of an FcγRIIa-blocking antibody IV.3 (10 μg/mL) relative to SFLLRN alone. AUC, area under the curve. Representative patterns of procoagulant platelet response demonstrating (C) classical VITT, (D) heparin-enhancing VITT and (E) negative profile. Procoagulant platelet response of individual patients in the development cohort are shown in panels F, G and H. Dotted horizontal line represents no plasma baseline.

FIG. 12. PF4 enhanced procoagulant platelet flow cytometry. (A) Plasma samples from 6 VITT positive and 8 VITT negative patients previously tested on the standard flow cytometry assay or (B) 15 individuals classified as negative, inconclusive or equivocal on the standard assay was retested on the platelet factor 4 (PF4) enhanced assay. Blue line represents a patient with heparin-enhancing response. Donor whole blood was treated with platelet agonist 5 μM SFLLRN and patient plasma in presence of 25 μg/mL purified native human PF4, unfractionated heparin (0.5 U/mL or 100 U/mL) and/or FcγRIIa-blocking antibody IV.3 (10 μg/mL). Dotted horizontal line represents 1.7-fold increase above no plasma baseline determined in FIG. 11 as the optimal cut-off for VITT. The procoagulant platelet response induced by VITT positive plasma was correlated with (C) anti-PF4 antibody titre represented by ELISA optical density (OD) values (n=87), (D) patient platelet count at the time of testing (n=98) and (E) D-dimer levels represented by fold-change above upper limit of normal (n=96). All patients recorded a platelet count nadir of <150×10⁹/L. Spearman correlation analysis was performed.

FIG. 13. Plasma from APS patient increases healthy donor procoagulant platelet response to thrombin stimulation. Citrated whole blood samples from healthy donors were treated with plasma from APS patients (n=4-5) or autologous plasma (n=5-6) in presence of (A) SFLLRN, (B) thrombin or (C) thrombin plus CRP-xL. Procoagulant platelets were measured by flow cytometry. Unpaired t-test was performed. Horizontal bars represent mean. *p<0.05; ns, not significant. Change in procoagulant platelet response is expressed as the difference in procoagulant platelet proportions between samples containing patient or autologous plasma, and no plasma baseline. Plasma from patients with APS increased the procoagulant platelet proportions in healthy donor platelets in the presence of thrombin stimulation by 3.8 to 8.0% relative to no plasma, compared to 0.3-5.5% increase in procoagulant platelet proportions induced by autologous plasma. No significant change in procoagulant response between patient and autologous plasma was observed in presence of SFLLRN or thrombin plus CRP-xL stimulation.

FIG. 14. Plasma from a patient with APS collected during plasma exchange (PLEX) increases the procoagulant platelet response to thrombin stimulation in healthy donor platelets. This increase in procoagulant platelet response is reduced in healthy platelets exposed to patient plasma post plasma exchange (Post-PLEX). Citrated whole blood samples from a healthy donor were treated with or without plasma from an APS patient in presence of (A) SFLLRN (5 μM), (B) thrombin (1 U/mL) or (C) thrombin (1 U/mL) plus CRP-xL (2 μg/mL). Procoagulant platelets are defined as GSAO+/CD62P+ platelets as measured by flow cytometry. Plasma from a patient with APS collected during plasma exchange increases the procoagulant platelet response to thrombin and thrombin plus CRP-xL stimulation in healthy donor platelets by 4.5-fold and 2.7-fold, respectively. This increase in procoagulant platelet response is reduced by over 50% in healthy platelets exposed to patient plasma post plasma exchange. This reduction in procoagulant platelet response may reflect the lower levels of platelet-activating antibodies in the patient after therapeutic plasma exchange.

FIG. 15. Hypersensitivity to thrombin induced by APS patient plasma (collected from plasma exchange, PLEX) is not fully inhibited by a complement protein C5 inhibitor eculizumab. Citrated whole blood samples from a healthy donor were treated with plasma from an APS patient in presence of (A) SFLLRN (5 μM), (B) thrombin (1 U/mL) or (C) thrombin (1 U/mL) plus CRP-xL (2 μg/mL). In some experiments, donor blood was pre-treated with eculizumab (50 μg/mL) for 15 min at room temperature prior to exposure to patient plasma and thrombin stimulation. Procoagulant platelets are defined as GSAO+/CD62P+ platelets as measured by flow cytometry.

FIG. 16. Hypersensitivity to thrombin induced by APS patient plasma (collected post plasma exchange, Post-PLEX) is not fully inhibited by a complement protein C5 inhibitor eculizumab. Citrated whole blood samples from a healthy donor were treated with plasma from an APS patient in presence of (A) SFLLRN (5 μM), (B) thrombin (1 U/mL) or (C) thrombin (1 U/mL) plus CRP-xL (2 μg/mL). In some experiments, donor blood was pre-treated with eculizumab (50 μg/mL) for 15 min at room temperature prior to exposure to patient plasma and thrombin stimulation. Procoagulant platelets are defined as GSAO+/CD62P+ platelets as measured by flow cytometry.

### DEFINITIONS

As used in this application, the singular form “a”, “an” and “the” include plural references unless the context clearly dictates otherwise.

As used herein, the term “comprising” means “including.” Variations of the word “comprising”, such as “comprise” and “comprises,” have correspondingly varied meanings.

It will be understood that use the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a concentration of between 2 mg/mL and 10 mg/mL is inclusive of a concentration of 2 mg/mL and a concentration of 10 mg/mL.

The terms ‘subject’ and ‘patient’ may be used interchangeably and refer to a human or other mammal suffering from, at risk of suffering from, or suspected of suffering from, an immune-mediated or inflammatory prothrombotic condition.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

This specification refers to an increase in the proportion of procoagulant platelets by reference to ‘fold-change’. For example, the proportion of procoagulant platelets in the combined sample (healthy donor whole blood plus subject plasma) may be increased by 1.1-fold compared to the healthy control. This means that the proportion of procoagulant platelets in the combined sample is 110% of the proportion of procoagulant platelets in the healthy control sample. That is, the combined sample may have a proportion of procoagulant platelets of 55%, while the healthy control sample may have a proportion of procoagulant platelets of 50%, a 1.1-fold, or 110% increase. In another example, the proportion of procoagulant platelets in the combined sample (healthy donor whole blood plus subject plasma) may be reduced to 0.9-fold of the value of the healthy control. This means that the proportion of procoagulant platelets in the combined sample is 90% of the proportion of procoagulant platelets in the healthy control sample. That is, the combined sample may have a proportion of procoagulant platelets of 50%, while the healthy control sample may have a proportion of procoagulant platelets of 55%. For the avoidance of doubt, a ‘greater’ decrease refers to a larger reduction in the proportion of procoagulant platelets. For example, a decrease to 0.5-fold is a greater decrease than a decrease to 0.9-fold.

This specification refers to several different control or confirmatory samples. For convenience, these are defined below as follows:
Assay control sample—the sample to be measured contacted with GSCA (4-(N—((S-glutathionyl)acetyl)amino)benzoic acid) and a control for the alpha granule detection agent. For example, if the proportion of procoagulant platelets in the healthy control sample is to be measured, an assay control sample is prepared comprising the healthy control sample, GSCA and a control for the alpha granule detection agent. This is a control for the procoagulant platelet assay itself.
Healthy control sample—healthy donor whole blood contacted with agonist. The healthy donor control sample does not contain subject plasma, but may contain plasma from a healthy donor. This is a control for the diagnosis of prothrombotic conditions including antiphospholipid syndrome and VITT. In this specification the healthy control sample does not contain healthy donor autologous plasma unless stated otherwise.
No heparin control sample—combined sample (healthy donor whole blood and patient plasma) contacted with agonist and no heparin. This is a control for the diagnosis of HIT.
Therapeutic heparin sample—combined sample (healthy donor whole blood and patient plasma) contacted with agonist and therapeutic concentration heparin. This is a confirmatory sample for the diagnosis of HIT and VITT.
High heparin control sample—combined sample (healthy donor whole blood plus subject plasma) contacted with agonist and high concentration heparin. This is a confirmatory sample for the diagnosis of HIT and VITT.

### DESCRIPTION OF EMBODIMENTS

A prothrombotic condition is one in which a subject is at increased risk of developing thrombosis. Prothrombotic conditions may be immune-mediated or inflammatory, meaning that thrombosis is caused by the action, either direct or indirect, of inflammatory or immune mediators (such as cytokines or antibodies) on platelets. Such inflammatory or immune mediators are found in the blood plasma. The inventors have surprisingly found that contacting the plasma of a subject suffering from an immune-mediated or inflammatory prothrombotic condition, or a subject at risk of suffering from a thrombotic event due to an immune-mediated or inflammatory prothrombotic condition, with whole blood obtained from a healthy donor, causes the platelets in the healthy donor whole blood to become procoagulant.

The inventors have also surprisingly used this method to develop highly sensitive and specific diagnostic tests, and to develop new diagnostic methods for prothrombotic conditions, including immune-mediated or inflammatory prothrombotic conditions, on the basis of procoagulant platelet generation. As the methods developed by the inventors utilise flow cytometry to determine the generation of procoagulant platelets, the methods are rapid, simple, and can be performed using readily available instrumentation. Further, the methods do not require the use of washed or isolated platelets, as is the case for existing diagnostic methods for some prothrombotic conditions. The use of washed or isolated platelets requires their time-consuming and labour-intensive isolation from whole blood, thereby limiting utility. Isolation of platelets from whole blood also requires specific blood collection procedures, can result in the introduction of processing artefacts, and requires relatively large volumes of blood. Further, the use of whole blood allows procoagulant platelets to remain in a more physiological milieu, together with other components of the blood. This replicates native conditions more closely where platelets can productively interact with leukocytes and other constituents of whole blood.

The methods of the invention also do not require the addition of exogeneous PF4, which use of which has a number of disadvantages such as the variability introduced by the varying oligomerization state of PF4 which is influenced by method of preparation, and high cost. The inventors have surprisingly found that it is possible to prime platelets with an agonist at a level sufficient to release PF4, but insufficient to generate a significant procoagulant response in donor platelets, and this provides a platform in which the procoagulant response is dependent on presence of FcγRIIa dependent procoagulant antibodies in patient plasma, without requirement for additional PF4.

### 1. Diagnosis of Prothrombotic Conditions

In one aspect of the invention there is a method of diagnosing an immune-mediated or inflammatory prothrombotic condition in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition.

A prothrombotic condition is one in which a subject is at increased risk of developing thrombosis. The prothrombotic conditions may be immune-mediated or inflammatory, meaning that thrombosis is caused by the action, either direct or indirect, of inflammatory or immune mediators (such as cytokines or antibodies) on platelets.

Immune-mediated or inflammatory prothrombotic conditions which may be identified using methods of the invention include heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers. Preferably, the prothrombotic condition is selected from the group consisting of HIT, VITT, sepsis, anti-phospholipid syndrome, and viral pneumonia. Preferably, the prothrombotic condition is selected from the group consisting of HIT, VITT, sepsis, and anti-phospholipid syndrome. Preferably, the prothrombotic condition is HIT, VITT, or anti-phospholipid syndrome. Preferably, the immune-mediated or inflammatory prothrombotic condition is HIT. Preferably, the immune-mediated or inflammatory prothrombotic condition is VITT. Preferably, the immune-mediated or inflammatory prothrombotic condition is anti-phospholipid syndrome.

In order to perform the method of the invention, a sample of plasma is obtained from the subject. The plasma may be obtained using any known method. For example, the plasma may be citrated platelet-poor plasma. The plasma may be stored prior to use in the method. The plasma may be stored at reduced temperature prior to use in the method, for example at −80° C.

The plasma is then combined with a sample of whole blood obtained from a healthy donor to obtain a combined sample. The whole blood may be collected using any known method. For example, it may be collected from the antecubital fossa of healthy donors into 3.2% citrate tubes using a 21 G butterfly needle. Preferably, the initial 3 mL of blood may be discarded. A healthy donor is an individual who is not suffering from any immune-mediated prothrombotic condition, and not undergoing treatment with antiplatelet agents, anticoagulants, or non-steroidal anti-inflammatory drugs. The healthy donor must also not be pregnant.

If the immune-mediated or inflammatory prothrombotic condition is mediated by FcγRIIa then preferably, the healthy donor is a FcγRIIa high responder. If the immune-mediated or inflammatory prothrombotic condition is not mediated by FcγRIIa then the healthy donor may not be a FcγRIIa high responder. FcγRIIa responsiveness may be determined using light transmission aggregometry response to anti-CD9 monoclonal antibody ALB6 as described in Journal of Thrombosis and Haemostasis. 2016; 14(12):2548-2552. FcγRIIa high responders may be donors whose platelets respond to ALB6 stimulation with greater than 80% aggregation, with a time to initiation of aggregation of less than 180 seconds. For example HIT and VITT are both mediated by FcγRIIa and for diagnosis of these conditions the healthy donor is preferably a FcγRIIa high responder. APS is not mediated by FcγRIIa therefore the healthy donor may not be a FcγRIIa high responder.

The volume of whole blood required to perform the method of the invention is less than 100 μL. The volume of whole blood required to perform the method of the invention may be less than 75, 50, 25, 20, 10, or 5 μL. The volume of whole blood required to perform the method of the invention may be between 1-5 μL, or between 1-10, 1-20, 1-25, 1-50, or 1-100 μL. The volume of whole blood required to perform the method of the invention may be about 5, 10, 20, 25, 50, 75, or 100 μL.

The subject and the healthy donor may be human. Alternatively, the subject and the healthy donor may be non-human mammals. The subject may have been administered heparin, other anticoagulant or antiplatelet medications. The subject may be currently being administered heparin, other anticoagulant or antiplatelet medications.

The combined sample is then contacted with at least one agonist to provide the test sample. An agonist is a substance which can induce platelet activation. Examples of suitable agonists include cross-linked collagen-related peptide (CRP-xL), thrombin receptor-activating peptide (SFLLRN), adenosine diphosphate (ADP), and thrombin. CRP-xL may be collagen-related peptide cross-linked with N-succinimidyl 3-(2-pyridyldithio)propionate. Combinations of these agonists may also be used, such as a combination of CRP-xL and thrombin. In this specification, the combination of CRP-xL at 2 μg/mL and thrombin at 1 U/mL is referred to as T1CRP2. However, CRP-xL and thrombin may equally be combined at other concentrations. For example CRP-xL at any concentration between about 0.1 to 10 μg/mL, such as 0.1, 0.5, 1.0, 1.5, 2.0, 2.5, 3.0, 3.5, 4.0, 4.5, 5.0, 6.0, 7.0, 8.0, 9.0, or 10 μg/mL may be combined with thrombin at any concentration between about 0.1 U/mL to 5 U/mL, such as 0.1, 0.5, 1.0, 2.0, 2.5, 3.0, 3.5, 4.0, 4.5, or 5 U/mL.

The concentration of the agonist used will depend on the agonist selected. For example, a suitable concentration of CRP-xL is between about 0.5 to about 10 μg/mL, or about 2 μg/mL. A suitable concentration of thrombin is between about 0.1-10 U/mL, or about 1 U/mL. A suitable concentration of SFLLRN is between about 1 to about 10 μM, or about 5 μM. A suitable concentration of ADP is between about 0.5 to about 50 μM, or about 20 μM. A suitable concentration of CRP-xL combined with thrombin may include, for example, CRP-xL at any concentration between about 0.1 to 10 μg/mL, such as 0.1, 0.5, 1.0, 1.5, 2.0, 2.5, 3.0, 3.5, 4.0, 4.5, 5.0, 6.0, 7.0, 8.0, 9.0, or 10 μg/mL, optionally combined with thrombin at any concentration between about 0.1 U/mL to 5 U/mL, such as 0.1, 0.5, 1.0, 2.0, 2.5, 3.0, 3.5, 4.0, 4.5, or 5 U/mL. For example, CRP-xL at 2 μg/mL may be combined thrombin at 1 U/mL.

Certain agonists may provide improved sensitivity and/or specificity for identification of certain prothrombotic conditions. For example, when the prothrombotic condition is HIT, the agonist may be SFLLRN. For example, when the prothrombotic condition is VITT, the agonist may be SFLLRN or thrombin. When the prothrombotic condition is anti-phospholipid syndrome, the agonist may be thrombin.

The test sample may not contain any exogeneous PF4. In the method of the invention the agonist is provided at a level sufficient to release PF4, but insufficient to generate a significant procoagulant response in donor platelets, which provides a platform in which the procoagulant response is dependent on presence of FcγRIIa dependent procoagulant antibodies in patient plasma, without requirement for additional PF4.

After the combined sample is contacted with the appropriate agonist(s) to obtain the test sample, the proportion of procoagulant platelets in the test sample is determined. The proportion of procoagulant platelets in at least one control sample is also determined. Procoagulant platelets may be distinguished from other platelet subpopulations by their increased uptake of 4-(N—(S-glutathionylacetyl)amino)phenylarsonous acid (GSAO) and combined with increased surface expression of alpha granule protein, as indicated by increased uptake of or labelling with an alpha-granule detection agent, as discovered by the inventors and described in PCT/AU2015/000638 and J Thromb Haemost., 2018, 16(6):1198-1210. Typically, the GSAO is conjugated with a fluorescent label such as Alexa Fluor 647 or another suitable dye. Typically, the alpha-granule detection agent is also conjugated with an appropriate fluorescent label. However other types of labels, such as radiolabels or rare metal isotopes (e.g. lanthanides) may be used. The uptake of GSAO and the alpha-granule detection agent may conveniently be determined by flow cytometry. The alpha-granule detection agent is a substance which indicates increased surface expression of alpha granule protein, for example by increased uptake of the agent or labelling with the agent, which may be detected e.g. by fluorescence. The alpha-granule detection agent may be, for example, an anti-P-selectin antibody or an anti-TLT-1 antibody. Preferably the alpha-granule detection agent is an anti-P-selectin antibody. In an embodiment, the proportion of procoagulant platelets in the combined sample and in at least one control sample is determined by contacting the combined sample and the at least one control sample with GSAO and anti-P-selectin antibody, both of which are fluorescent-labelled, wherein platelets (cells which are CD41a+ and CD45−) having increased uptake of GSAO and increased binding of anti-P-selectin antibody are identified by flow cytometry.

The increased uptake of GSAO and increased surface expression of alpha granule protein in the sample being measured (e.g. the test sample or the at least one control sample, etc.) is determined in comparison to an assay control sample. The assay control sample provides a reference for the level of non-specific uptake of GSAO and alpha granule detection agent in the sample being measured. The assay control sample is obtained by contacting the sample to be measured (e.g. the combined sample, or the at least one control sample, etc.) with 4-(N—((S-glutathionyl) acetyl)amino)benzoic acid (GSCA) and a control for the alpha granule detection agent. As such, an assay control sample is prepared corresponding to each individual sample to be measured. GSCA is a control compound for GSAO (that is, it is identical to GSAO except the arsenoxide moiety is replaced with a carboxylic acid). If the alpha granule detection agent is an antibody, the corresponding control is an antibody isotype control. The GSCA and the control for the alpha granule detection agent are typically labelled, in the same manner as the GSAO and alpha granule detection agent. Platelets having increased uptake of GSAO and the alpha granule detection agent, compared to the uptake of GSCA and the control for the alpha granule detection agent in the assay control sample, are identified as procoagulant platelets. In a preferred embodiment, where the proportion of procoagulant platelets in the combined sample is determined by flow cytometry, appropriate fluorescence intensity thresholds (‘gates’) are set by the operator for each assay control sample, wherein if fluorescence levels exceed these gate levels, the platelet is counted as a procoagulant platelet. The fluorescence intensity values for the gates are dependent on the antibody label and will differ from one flow cytometer to another, as well as between individual runs and between different batches of labelled antibodies. A commonly used guideline is to set the gates such that 0.5% or less of events for the assay control sample fall outside each gate. These gates are then applied to the sample to be measured (e.g. test sample) contacted with GSAO and alpha granule detection agent to determine the proportion of procoagulant platelets in the sample to be measured. That is, platelets having levels of GSAO uptake and surface alpha granule protein both exceeding the gate levels, are considered to have both increased uptake of GSAO and increased surface expression of alpha granule protein compared to the assay control sample, such that they are identified as procoagulant platelets.

Once the proportion of procoagulant platelets in the combined sample and in the at least one control sample is determined, these values can be compared to determine whether the subject is suffering from a prothrombotic condition. An increased proportion of procoagulant platelets in the test sample compared to the at least one control sample indicates that the subject is suffering from a prothrombotic condition. The proportion of procoagulant platelets in the test sample may additionally be compared with the proportion of procoagulant platelets in a control sample comprising healthy donor whole blood and the agonist, as well as plasma from a healthy donor (healthy control sample including plasma from a healthy donor, i.e. autologous plasma).

The increased proportion of procoagulant platelets in the test sample compared to the at least one control sample which indicates that the subject is suffering from a prothrombotic condition may be an at least about 1.1-fold increase, or an at least about 1.15, 1.2, 1.25, 1.5, 2, 3, 4, 5, 6, 7, 8, 9, 10, 20, 30, 40, 50, 60, 70, 80, 90, or about 100-fold increase. The increase may be between about 1.1 to 100-fold, or between about 1.2, 1.25, 1.5, 2, 3, 4, 5, 6, 7, 8, 9, 10, 20, 30, 40, or 50 to 100-fold. The amount by which the proportion of procoagulant platelets in the test sample is increased in a subject suffering from a prothrombotic condition, compared to the proportion of procoagulant platelets in the healthy control sample, may vary depending on the agonist with which the combined sample is contacted. However, regardless of the agonist used, the increase which is indicative of the subject suffering from an immune-mediated or inflammatory prothrombotic condition is typically at least 1.1-fold.

The method of the invention may provide a highly sensitive and specific test for whether a subject is suffering from an immune-mediated or inflammatory prothrombotic condition. Determination of whether a subject is suffering from an immune-mediated or inflammatory prothrombotic condition using the method according to the invention may yield a Receiver Operator Curve (ROC) area under the curve (AUC) of 1, demonstrating 100% sensitivity and specificity. The method of the invention may yield a ROC AUC of at least about 0.8, or at least about 0.85, 0.9, 0.95, 0.98, or 0.99. The method of the invention may exhibit a sensitivity and specificity of at least about 80%, or at least about 85, 90, 95, 98, 99, or 100%.

The method of the invention is carried out in vitro.

### 1.1 Heparin-Induced Thrombocytopenia (HIT)

In one embodiment of the invention, there is provided a method of diagnosing heparin-induced thrombocytopenia (HIT) in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from HIT, wherein an increased proportion of procoagulant platelets in the test sample compared to the at least one control sample indicates that the subject is suffering from HIT.

In health, procoagulant platelet formation occurs after platelet stimulation with a combination of strong agonists: thrombin and stimulation of the major platelet collagen receptor, glycoprotein VI (GPVI). Little procoagulant platelet formation occurs with weaker agonists such as adenosine diphosphate (ADP). The inventors have surprisingly found that in the presence of plasma from a subject suffering from HIT, platelets from healthy donor blood displayed a heparin-dependent sensitisation to procoagulant platelet formation following stimulation by a range of receptor-specific platelet agonists traditionally considered weak agonists.

When the prothrombotic condition is HIT, the test sample may also be contacted with heparin. As discussed above, key to the pathogenesis of HIT is the formation of platelet-activating immunoglobulin G (IgG) antibodies recognising complexes of the platelet alpha granule protein, PF4, and heparin. The complex of PF4 and heparin only forms at a particular stoichiometry, and if either is in excess, the complex does not form and FcγRIIa is not activated (and procoagulant platelets are not generated). The presence of HIT may therefore be confirmed by contacting the test sample with a therapeutic concentration of heparin (which should form a complex with PF4, leading to FcγRIIa activation, and an increase in procoagulant platelets), and by separately contacting the test sample with a high concentration of heparin (which should not form a complex with PF4, leading to no increase in procoagulant platelets). Contacting the test sample with a high concentration of heparin also constitutes the high heparin control sample, discussed below. A therapeutic concentration of heparin is between about 0.1 and about 0.7 U/mL, or between about 0.3 and about 0.7 U/mL, or about 0.5 U/mL. A high concentration of heparin is about 50 to 200 U/mL, or about 100 U/mL.

Therefore, in a method for diagnosing HIT in a subject, the at least one control sample comprises a no heparin control sample and a high heparin control sample. Alternatively, in some embodiments, the at least one control sample comprises a no heparin control sample. Alternatively, in some embodiments the at least one control sample comprises a high heparin control sample. The no heparin control sample comprises, or consists of, the combined sample and the agonist (the test sample), and no heparin. The high heparin control sample comprises, or consists of, the combined sample and the agonist (the test sample), and high concentration heparin. A therapeutic concentration of heparin is between about 0.1 and about 0.7 U/mL, or between about 0.3 and about 0.7 U/mL, or about 0.5 U/mL. A high concentration of heparin is about 50 to 200 U/mL, or about 100 U/mL. Thus in a method of diagnosing HIT in a subject, the proportion of procoagulant platelets in (i) the test sample which is further contacted with therapeutic concentration heparin, (ii) the no heparin control sample, and (iii) the high heparin control sample is determined. An increased proportion of procoagulant platelets in the test sample which is further contacted with therapeutic concentration heparin, compared with the proportion of procoagulant platelets in both the no heparin control sample and the high heparin control sample, indicates that the subject is suffering from HIT.

The heparin-dependence of the proportion of procoagulant platelets in samples from subjects suffering from HIT allows this condition to be identified with particularly high sensitivity and specificity. Advantageously, the method is capable of differentiating a subject suffering from HIT from a subject suffering from acute thrombocytopenia which is not heparin-induced. In particular, wherein the agonist is SFLLRN the method of the invention may identify that a subject is suffering from HIT with a Receiver Operator Curve (ROC) area under the curve (AUC) of 1, demonstrating 100% sensitivity and specificity. This embodiment of the method of the invention may yield a ROC AUC of at least about 0.8, or at least about 0.85, 0.9, 0.95, 0.98, or 0.99. This embodiment of the method of the invention may exhibit a sensitivity and specificity of at least about 80%, or at least about 85, 90, 95, 98, 99, or 100%.

The increased proportion of procoagulant platelets in the test sample contacted with therapeutic concentration heparin compared to the no heparin control sample and the high heparin control sample which indicates that the subject is suffering from HIT may be an at least about 1.1-fold increase, or an at least about 1.15, 1.2, 1.25, 1.5, 2, 3, 4, 5, 6, 7, 8, 9, 10, 20, 30, 40, 50, 60, 70, 80, 90, or about 100-fold increase. The increase may be between about 1.1 to 100-fold, or between about 1.2, 1.25, 1.5, 2, 3, 4, 5, 6, 7, 8, 9, 10, 20, 30, 40, or 50 to 100-fold. That is, a diagnosis of HIT may be made when the proportion of procoagulant platelets in the test sample contacted with therapeutic concentration heparin is increased by at least 1.1-fold compared to the no heparin control sample, and the proportion of procoagulant platelets in the test sample contacted with therapeutic concentration heparin is also increased by at least 1.1-fold compared to the high heparin control sample,

The amount by which the proportion of procoagulant platelets in the test sample is increased in a subject suffering from HIT, compared to the proportion of procoagulant platelets in the no heparin and high heparin control samples, may vary depending on the agonist with which the combined sample is contacted. For example, if the agonist is CRP-xL, the proportion of procoagulant platelets in the test sample contacted with therapeutic concentration heparin compared to the no heparin control sample which indicates that the subject is suffering from HIT may be an at least about 1.1-fold increase, or an increase of between about 1.1 and 5-fold, the proportion of procoagulant platelets in the test sample contacted with therapeutic concentration heparin compared to the high heparin control sample which indicates that the subject is suffering from HIT may be an at least about 2-fold increase, or an increase of between about 1.5 and 5-fold.

If the agonist is SFLLRN, the proportion of procoagulant platelets in the test sample contacted with therapeutic concentration heparin compared to the no heparin control sample which indicates that the subject is suffering from HIT may be an at least about 3-fold increase, or an increase of between about 2 and 10-fold, and the proportion of procoagulant platelets in the test sample contacted with therapeutic concentration heparin compared to the high heparin control sample which indicates that the subject is suffering from HIT may be an at least about 7-fold increase, or an increase of between about 2 and 20-fold.

If the agonist is ADP, the proportion of procoagulant platelets in the test sample contacted with therapeutic concentration heparin compared to the no heparin control sample which indicates that the subject is suffering from HIT may be an at least about 3-fold increase, or an increase of between about 2 and 12-fold, and the proportion of procoagulant platelets in the test sample contacted with therapeutic concentration heparin compared to the high heparin control sample which indicates that the subject is suffering from HIT may be an at least about 4-fold increase, or an increase of between about 2 and 14-fold.

In the diagnosis of HIT, if the agonist is a combination of CRP-xL and thrombin, the at least one control sample is a healthy control sample and the test sample is not contacted with heparin. When the at least one agonist includes thrombin, it is not possible to measure the heparin-dependent formation of procoagulant platelets due to the ex vivo inhibition of thrombin by heparin. The healthy control sample comprises, or consists of, healthy donor whole blood and the agonist, and does not contain patient plasma. The healthy control sample may optionally also comprise plasma from a healthy donor (i.e. autologous plasma). The proportion of procoagulant platelets in the test sample compared to the healthy control sample which indicates that the subject is suffering from HIT may be an at least about 2-fold increase, or an increase of between about 1.1 and 10-fold.

However, regardless of the agonist used, the increase which is indicative of the subject suffering from a prothrombotic condition, such as HIT, is typically at least 1.1-fold.

Thus in one embodiment, there is provided a method of diagnosing HIT in a subject, the method comprising combining a sample of plasma obtained from the subject with a sample of whole blood obtained from a healthy donor, optionally a FcγRIIa high responder, to obtain a combined sample. The combined sample is then contacted with an agonist, which is preferably SFLLRN, to provide a test sample. The test sample is then contacted with therapeutic concentration heparin. The proportion of procoagulant platelets in the test sample contacted with therapeutic concentration heparin, as well as in a no heparin control sample and in a high heparin control sample, is determined by contacting each sample with GSAO and an anti-P-selectin antibody, wherein platelets in the combined sample which have both increased uptake of GSAO and increased expression of P-selectin compared to corresponding assay control samples are identified as procoagulant platelets. If the proportion of procoagulant platelets in the test sample contacted with therapeutic concentration heparin compared to the proportion of procoagulant platelets in the no heparin control sample and in the high heparin control sample is increased at least about 1.1-fold, or between about 1.1- and about 100-fold, then the subject is identified as suffering from HIT. More preferably, if the proportion of procoagulant platelets in the test sample contacted with therapeutic concentration heparin compared to proportion of procoagulant platelets in the no heparin control sample is increased at least about 3-fold, or between about 2 and 10-fold, and the proportion of procoagulant platelets in the test sample contacted with therapeutic concentration heparin compared the proportion of procoagulant platelets in the high heparin control sample is increased at least 7-fold, or between about 2 and 20-fold, then the subject is identified as suffering from HIT. In this embodiment, the corresponding assay control samples are obtained by contacting the test sample plus therapeutic concentration heparin, no heparin control sample, and high heparin control sample, with GSCA and an antibody isotype control for the P-selectin antibody. The no heparin control sample comprises the combined sample and SFLLRN (the test sample), and no heparin, and the high heparin control sample comprises the combined sample and SFLLRN (the test sample), and high concentration heparin. The therapeutic concentration of heparin is between about 0.1 and about 0.7 U/mL, preferably 0.5 U/mL and the high concentration of heparin is between about 50 to 200 U/mL, preferably 100 U/mL.

### 1.2 Vaccine-Induced Thrombotic Thrombocytopenia (VITT)

In one embodiment of the invention, there is provided a method of diagnosing vaccine-induced thrombotic thrombocytopenia (VITT) in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from VITT, wherein an increased proportion of procoagulant platelets in the test sample compared to the at least one control sample indicates that the subject is suffering from VITT.

In this embodiment, the subject may have been administered a vaccine and is suspected of suffering from VITT. The vaccine may be recombinant adenoviral vector vaccine, for example a recombinant adenoviral vector encoding the spike protein antigen of SARS-CoV-2. The vaccine may be, for example, the AstraZeneca AZD1222 vaccine against COVID-19 (trade name Vaxzevria), the Johnson & Johnson/Janssen JNJ-78436735 vaccine against COVID-19, the CanSino Biologics AD5-nCOV vaccine against COVID-19 (trade name Convidecia), or the Gamaleya Research Institute of Epidemiology and Microbiology Gam-COVID-Vac vaccine against COVID-19 (trade name Sputnik V). In one embodiment, the vaccine is the AstraZeneca AZD1222 vaccine against COVID-19 (trade name Vaxzevria).

Certain agonists may provide improved sensitivity and/or specificity for identification of VITT. For example, the combination of SFLLRN with either heparin (at a therapeutic and/or a high concentration), thrombin, AZD1222 vaccine, or the SARS-CoV-2 spike protein. In particular, comparison of the proportion of procoagulant platelets in samples contacted with different agonists allows for improved sensitivity and/or specificity for identification of VITT.

In an exemplary embodiment, where AZD1222 vaccine is the antagonist, it may be diluted 1 in 2000. In another exemplary embodiment, where SARS-CoV-2 spike protein is the antagonist it may be provided be at a concentration 20 μg/mL.

In another embodiment, suitable agonists include thrombin receptor-activating peptide (SFLLRN) and thrombin. The test sample may comprise SFLLRN at a concentration between about 0.1 to 10 μM, or between about 0.1 to 0.5, 0.1 to 1, 0.1 to 2, 0.1 to 3, 0.1 to 4, 0.1 to 5, 0.1 to 6, 0.1 to 7, 0.1 to 8, 0.1 to 9, 0.5 to 1, 0.5 to 2, 0.5 to 3, 0.5 to 4, 0.5 to 5, 0.5 to 6, 0.5 to 7, 0.5 to 8, 0.5 to 9, 0.5 to 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 μM. The test sample may comprise SFLLRN at a concentration of about 0.1 μM, or about 0.5, 1, 2, 3, 4, 5, 6, 7, 8, 9, or about 10 μM. The test sample may comprise thrombin at a concentration between about 0.1 to 5 U/mL, or between about 0.1 to 0.5, 0.1 to 1, 0.1 to 2, 0.1 to 3, 0.1 to 4, 0.5 to 1, 0.5 to 2, 0.5 to 3, 0.5 to 4, 0.5 to 5, 1 to 2, 1 to 3, 1 to 4, 1 to 5, 2 to 3, 2 to 4, 2 to 5, 3 to 4, 3 to 5, or 4 to 5 U/mL. The test sample may comprise thrombin at a concentration of about 0.1 U/mL, or about 0.5, 1, 2, 3, 4, or about 5 U/mL.

When the prothrombotic condition is VITT, the test sample may also be contacted with heparin. The inventors have found that plasma samples from confirmed (ELISA-positive and SRA-positive) VITT patients induced an FcγRIIa dependent procoagulant platelet response in healthy donor platelets that is suppressible by high dose heparin. The inclusion of low-dose heparin was found to diminish the procoagulant platelet response induced by VITT plasma in most but not all patients. Without wishing to be bound by theory, the inventors consider that the addition of heparin presumably competes with VITT antibodies for PF4 binding, resulting in an abrogated platelet-activating effect with complete abrogation at high-dose heparin.

### 1.2.1 SFLLRN Agonist

When the agonist is SFLLRN, the proportion of procoagulant platelets is determined in the test sample, a healthy control sample, a therapeutic heparin sample, and a high heparin sample. As previously defined, the test sample comprises, or consists of, the combined sample and the agonist. The healthy control sample comprises, or consists of, healthy donor whole blood contacted with the agonist, but does not contain subject plasma. The therapeutic heparin sample comprises, or consists of, the combined sample (healthy donor whole blood and patient plasma) contacted with the agonist and therapeutic concentration heparin. The high heparin sample comprises, or consists of, the combined sample (healthy donor whole blood and patient plasma) contacted with the agonist and high concentration heparin. A therapeutic concentration of heparin is between about 0.1 and about 0.7 U/mL, or between about 0.3 and about 0.7 U/mL, or about 0.5 U/mL. A high concentration of heparin is about 50 to 200 U/mL, or about 100 U/mL.

Once the proportion of procoagulant platelets in the test sample, healthy control sample, therapeutic heparin sample, and high heparin sample has been determined, these values can be compared to determine whether the subject is suffering from VITT. An increased proportion of procoagulant platelets in the test sample, therapeutic heparin sample, and high heparin sample, compared to the healthy control sample, indicates that the subject is suffering from VITT.

The increased proportion of procoagulant platelets in the test sample, low heparin sample, and high heparin sample, compared to the healthy sample which indicates that the subject is suffering from VITT may be an at least about 1.1-fold increase, or an at least about 1.15, 1.2, 1.25, 1.5, 2, 3, 4, 5, 6, 7, 8, 9, 10, 20, 30, 40, 50, 60, 70, 80, 90, or about 100-fold increase. The increase may be between about 1.1 to 100-fold, or between about 1.2, 1.25, 1.5, 2, 3, 4, 5, 6, 7, 8, 9, 10, 20, 30, 40, or 50 to 100-fold. The amount by which the proportion of procoagulant platelets in the test sample, low heparin sample, and high heparin sample is increased in a subject suffering from VITT, compared to the proportion of procoagulant platelets in the healthy control sample, may vary depending on the agonist with which the combined sample is contacted. However, regardless of the agonist used, the increase which is indicative of the subject suffering from VITT is typically at least 1.1-fold.

Observation of differences in the increased proportion of procoagulant platelets in the test sample, low heparin sample, and high heparin sample, compared to the healthy control sample enables the method of the invention to distinguish classical VITT, heparin-enhancing VITT, and a negative result (i.e. subject is not suffering from VITT). Heparin-enhancing VITT is likely to be exacerbated by exposure to heparin or low molecular weight heparin and patients with heparin-enhancing VITT should avoid heparin drugs lifelong. Heparins should also be avoided in classical VITT, however with further clinical follow up patients with classical VITT may not need to avoid heparin in future. The smaller increase in procoagulant platelet proportion in the therapeutic heparin sample and the high heparin sample compared to the test sample allows the method of the invention to identify classical VITT. A larger increase in procoagulant platelet proportion in the therapeutic heparin sample compared to the test sample or the high heparin sample allows the method of the invention to identify heparin-enhancing VITT. Advantageously, the method is capable of differentiating a subject suffering from VITT with platelet-activating antibodies from a subject suffering from acute thrombocytopenia or thrombosis which is not associated with a vaccine.

Additionally, the test sample may be further contacted with the monoclonal antibody IV.3. Comparison of the proportion of procoagulant platelets in the test sample, compared to the test sample contacted with IV.3 can improve confidence that the subject is suffering from VITT. A decrease in the proportion of procoagulant platelets in the test sample contacted with IV.3 compared to the test sample (without IV.3) confirms that the subject is suffering from VITT. The decrease in the proportion of procoagulant platelets may be a decrease to about 0.9-fold, or greater, i.e. to about 0.8, 0.7, 0.6, 0.5, 0.4, 0.3, 0.2, 0.1, or 0.05-fold.

Additionally, the test sample may be further contacted with IVIg. Comparison of the proportion of procoagulant platelets in the test sample, compared to the test sample contacted with IVIg can improve confidence that the subject is suffering from VITT. A decrease in the proportion of procoagulant platelets in the test sample contacted with IVIg compared to the test sample (without IVIg) confirms that the subject is suffering from VITT. The decrease in the proportion of procoagulant platelets may be a decrease to about 0.9-fold, or greater, i.e. to about 0.8, 0.7, 0.6, 0.5, 0.4, 0.3, 0.2, 0.1, or 0.05-fold.

Specifically, the following comparisons may be made when the agonist is SFLLRN. When the compared to the healthy control sample, an increase in the proportion of procoagulant platelets in the test sample of greater than 1.7-fold, a change in the proportion of procoagulant platelets in the therapeutic heparin sample of up to 1.5-fold, and a change in the proportion of procoagulant platelets in the high heparin sample of up to 1.5-fold, indicates that the subject is suffering from classical VITT. Compared to the healthy control sample, an increase in the proportion of procoagulant platelets in the test sample of greater than 1-fold, an increase in the proportion of procoagulant platelets in the therapeutic heparin sample of greater than 1.7-fold, and a change in the proportion of procoagulant platelets in the high heparin sample of up to 1.5-fold, indicates that the subject is suffering from heparin-enhancing VITT. Compared to the healthy control sample, a change in the proportion of procoagulant platelets in the test sample of up to 1.5-fold, a change in the proportion of procoagulant platelets in the therapeutic heparin sample of up to 1.5-fold, and a change in the proportion of procoagulant platelets in the high heparin sample of up to 1.5-fold, indicates that the subject is not suffering from VITT.

When the agonist is SFLLRN and the test sample is further contacted with IV.3, and when compared to the test sample, a decrease in the proportion of procoagulant platelets in the test sample further contacted with the monoclonal antibody IV.3 to 0.7-fold or greater (i.e. the proportion of procoagulant platelets is reduced by at least 30% in presence of IV.3, compared to the test sample without IV.3) indicates that the subject is suffering from classical VITT or heparin-enhancing VITT, and a decrease in the proportion of procoagulant platelets in the test sample further contacted with the monoclonal antibody IV.3 to less than 0.7-fold (i.e. the proportion of procoagulant platelets is reduced by less than 30% in presence of IV.3, compared to the test sample without IV.3) indicates that the subject is not suffering from VITT.

The above comparisons are summarised in Table 1 below.

TABLE 1

| | Fold- | | | |
|---|---|---|---|---|
| | increase in | Fold- | | |
| Fold- | proportion of | increase in | | |
| increase in | procoagulant | proportion of | Fold- | |
| proportion of | platelets in | procoagulant | change in | |
| procoagulant | therapeutic | platelets in | proportion of | |
| platelets in | heparin | high heparin | procoagulant | |
| test sample | sample | sample | platelets in | |
| compared to | compared to | compared to | test sample + | |
| healthy | healthy | healthy | IV.3 | |
| control | control | control | compared to | |
| sample | sample | sample | test sample | Conclusion |
| >1.7 | <1.5 | <1.5 | <0.7 | Classical |
| | | | | VITT |
| >1 | >1.7 | <1.5 | <0.7 | heparin- |
| | | | | enhancing |
| | | | | VITT |
| <1.5 | <1.5 | <1.5 | Any ratio | Negative |

### 1.2.2 Thrombin Agonist

Where the agonist is thrombin, the proportion of procoagulant platelets is determined in the test sample, and a healthy control sample, as previously defined. The proportion of procoagulant platelets is determined in the same manner as described above. Once the proportion of procoagulant platelets in the test sample and healthy control sample has been determined, these values can be compared to determine whether the subject is suffering from VITT. An increased proportion of procoagulant platelets in the test sample compared to the healthy control sample, indicates that the subject is suffering from VITT. When the at least one agonist includes thrombin, it is not possible to measure the heparin-dependent formation of procoagulant platelets due to the ex vivo inhibition of thrombin by heparin.

The increased proportion of procoagulant platelets in the test sample compared to the healthy sample which indicates that the subject is suffering from VITT may be an at least about 1.1-fold increase, or an at least about 1.15, 1.2, 1.25, 1.5, 2, 3, 4, 5, 6, 7, 8, 9, 10, 20, 30, 40, 50, 60, 70, 80, 90, or about 100-fold increase. The increase may be between about 1.1 to 100-fold, or between about 1.2, 1.25, 1.5, 2, 3, 4, 5, 6, 7, 8, 9, 10, 20, 30, 40, or 50 to 100-fold. The increase may be, for example, 2-fold. The amount by which the proportion of procoagulant platelets in the test sample, low heparin sample, and high heparin sample is increased in a subject suffering from VITT, compared to the proportion of procoagulant platelets in the healthy control sample, may vary depending on the agonist with which the combined sample is contacted. However, regardless of the agonist used, the increase which is indicative of the subject suffering from VITT is typically at least 1.1-fold.

### 1.2.3 Selectivity and Specificity

The method of the invention may provide a highly sensitive and specific test for whether a subject is suffering from VITT. Determination of whether a subject is suffering from VITT using the method according to the invention may yield a Receiver Operator Curve (ROC) area under the curve (AUC) of 0.9830, demonstrating 100% specificity and 81.48% sensitivity at a cut-off value of 3.595%, or 79.17% specificity and 100% sensitivity at a cut-off value of 6.065%. The method of the invention may yield a ROC AUC of at least about 0.8, or at least about 0.85, 0.9, 0.95, 0.98, or 0.99. The method of the invention may exhibit a sensitivity and specificity of at least about 80%, or at least about 85, 90, 95, 98, 99, or 100%.

### 1.3 Anti-Phospholipid Syndrome (APS)

In one embodiment of the invention, there is provided a method of diagnosing anti-phospholipid syndrome (APS) in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from APS, wherein an increased proportion of procoagulant platelets in the test sample compared to the at least one control sample indicates that the subject is suffering from APS.

In this embodiment, the at least one control sample comprises a healthy control sample, which comprises healthy donor whole blood contacted with the agonist, and does not comprise subject plasma. The healthy control sample may optionally comprise healthy donor plasma, i.e. autologous plasma.

When the prothrombotic condition is APS, the agonist may preferably be thrombin. Alternatively, the agonist may be CRP-xL, SFLLRN, ADP or CRP-xL combined with thrombin. The proportion of procoagulant platelets in the combined sample compared to the healthy control sample (with or without healthy donor autologous plasma) which indicates that the subject is suffering from anti-phospholipid syndrome may be an at least about 1.1-fold increase, or an increase of between about 1.1 and 15-fold, or an increase of between about 1.1-2, 1.1-3, 1.1-4, 1.1-5, 1.1-6, 1.1-7, 1.1-8, 1.1-9, 1-10, 1-11, 1-12, 1-13, 1-14,2-3, 2-4, 2-5, 2-6, 2-7, 2-8, 2-9, 2-10, 2-11-, 2-12, 2-13, 2-14, 2-15, 3-4, 3-5, 3-6, 3-7, 3-8, 3-9, 3-10, 3-11, 3-12, 3-13, 3-14, 3-15, 4-5, 4-6, 4-7, 4-8, 4-9, 4-10, 4-11, 4-12, 4-1, 4-14, 4-15, 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 12-15, 13-14, 13-15, or 14-15-fold.

1.4 Viral Pneumonia e.g. COVID-19

In one embodiment of the invention, there is provided a method of diagnosing viral pneumonia in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from viral pneumonia, wherein an increased proportion of procoagulant platelets in the test sample compared to the at least one control sample indicates that the subject is suffering from viral pneumonia.

Viral pneumonia, including COVID-19, is caused by an inflammatory response to infection with a virus, e.g. SARS-CoV-2, and can lead to an increased risk of thrombosis. For example, in severe SARS-CoV-2 infection (involving ICU admission and ventilatory support) platelet dynamics reveal a small but highly activated platelet subpopulation linked with shorter clot formation times, increased maximal clot firmness, and reduced maximal clot lysis, which correlates with neutrophil counts and illness severity.

In this embodiment, the at least one control sample comprises a healthy control sample, which comprises healthy donor whole blood contacted with the agonist, and does not comprise patient plasma.

When the prothrombotic condition is viral pneumonia, the agonist may preferably be CRP-xL combined with thrombin. Alternatively, the agonist may be CRP-xL, SFLLRN, ADP or CRP-xL combined with thrombin. The proportion of procoagulant platelets in the combined sample compared to the healthy control sample (with or without healthy donor autologous plasma) which indicates that the subject is suffering from viral pneumonia at risk of thrombosis may be an at least about 1.1-fold increase, or an increase of between about 1.1 and 10-fold, or an increase of between about 1.1-2, 1.1-3, 1.1-4, 1.1-5, 1.1-6, 1.1-7, 1.1-8, 1.1-9, 1-10, 1-11, 1-12, 1-13, 1-14,2-3, 2-4, 2-5, 2-6, 2-7, 2-8, 2-9, 2-10, 2-11-, 2-12, 2-13, 2-14, 2-15, 3-4, 3-5, 3-6, 3-7, 3-8, 3-9, 3-10, 3-11, 3-12, 3-13, 3-14, 3-15, 4-5, 4-6, 4-7, 4-8, 4-9, 4-10, 4-11, 4-12, 4-1, 4-14, 4-15, 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 12-15, 13-14, 13-15, or 14-15-fold.

### 1.5 Sepsis

In one embodiment of the invention, there is provided a method of diagnosing sepsis in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from sepsis, wherein an increased proportion of procoagulant platelets in the test sample compared to the at least one control sample indicates that the subject is suffering from sepsis.

Sepsis results in platelet activation and increased risk of thrombosis.

In this embodiment, the at least one control sample comprises a healthy control sample, which comprises healthy donor whole blood contacted with the agonist, and does not comprise patient plasma.

When the immune-mediated or inflammatory prothrombotic condition is sepsis, the agonist may preferably be CRP-xL combined with thrombin. Alternatively, the agonist may be CRP-xL, SFLLRN, ADP or CRP-xL combined with thrombin. The proportion of procoagulant platelets in the combined sample compared to the healthy control sample (with or without healthy donor autologous plasma) which indicates that the subject is suffering from sepsis at risk of thrombosis may be an at least about 1.1-fold increase, or an increase of between about 1.1 and 10-fold, or an increase of between about 1.1-2, 1.1-3, 1.1-4, 1.1-5, 1.1-6, 1.1-7, 1.1-8, 1.1-9, 1-10, 1-11, 1-12, 1-13, 1-14,2-3, 2-4, 2-5, 2-6, 2-7, 2-8, 2-9, 2-10, 2-11-, 2-12, 2-13, 2-14, 2-15, 3-4, 3-5, 3-6, 3-7, 3-8, 3-9, 3-10, 3-11, 3-12, 3-13, 3-14, 3-15, 4-5, 4-6, 4-7, 4-8, 4-9, 4-10, 4-11, 4-12, 4-1, 4-14, 4-15, 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 12-15, 13-14, 13-15, or 14-15-fold.

2. Treatment Etc. Of Immune-Mediated or Inflammatory Prothrombotic Conditions

It is important to apply the appropriate treatment for a prothrombotic condition in view of the risk of side effects, such as major bleeding. Thus also disclosed herein is a method of treatment of an immune-mediated or inflammatory prothrombotic condition in a subject, which comprises determining if the subject is suffering from the immune-mediated or inflammatory prothrombotic condition in accordance with the method of diagnosis described in sections 1 to 1.5 above, and administering treatment for the immune-mediated or inflammatory prothrombotic condition to the subject, only if the subject is determined to be suffering from the immune-mediated or inflammatory prothrombotic condition.

The inventors have also surprisingly discovered that the proportion of procoagulant platelets in a sample derived from a subject suffering from an immune-mediated or inflammatory prothrombotic condition is increased when the subject is at increased risk of experiencing a thrombotic event. Thus also disclosed herein is a method of treatment of an immune-mediated or inflammatory prothrombotic condition in a subject, which comprises determining if the subject is at increased risk of experiencing a thrombotic event in accordance with the method of diagnosis described in sections 1 to 1.5 above, and administering or escalating treatment for the immune-mediated or inflammatory prothrombotic condition to the subject, only if the subject is determined to be at increased risk of suffering a thrombotic event.

Individual subjects may react differently to particular therapies for immune-mediated or inflammatory prothrombotic conditions. Thus also disclosed herein is a method of selecting a therapy for treatment of an immune-mediated or inflammatory prothrombotic condition in a subject, which comprises determining if the therapy causes a reduction in the proportion of procoagulant platelets in the subject. Whether the therapy causes a reduction in the proportion of procoagulant platelets in the subject is determined by comparing the proportion of procoagulant platelets in the test sample and in a therapy test sample (obtained by contacting the test sample with the therapy), determined in accordance with the method described in sections 1 to 1.5 above, and selecting the therapy for treatment of the immune-mediated or inflammatory prothrombotic condition if the proportion of procoagulant platelets in the therapy test sample is lower than the proportion of procoagulant platelets in the test sample.

In the method of selecting a therapy for treatment of an immune-mediated or inflammatory prothrombotic condition, the proportion of procoagulant platelets in the therapy test sample is lower than the proportion of procoagulant platelets in the test sample means that the proportion of procoagulant platelets in the therapy test sample is reduced to at least 0.99-fold compared to the proportion of procoagulant platelets in the therapy test sample is reduced to at least 0.95, 0.9, 0.8, 0.7, 0.6, 0.5, 0.4, 0.3, 0.2, 0.1, or 0.05-fold of the proportion of procoagulant platelets in the test sample. Where the proportion of procoagulant platelets in the therapy test sample is lower than the proportion of procoagulant platelets in the test sample this is understood to mean that the therapy is an effective therapy for the subject.

The method of selecting a therapy for treatment of an immune-mediated or inflammatory prothrombotic condition can further comprise contacting the test sample with varying concentrations of the therapy to obtain multiple therapy test samples, such that if a concentration-dependent reduction in the procoagulant platelets is observed, this can provide further confirmation of the efficacy of the therapy.

It can be beneficial to monitor the efficacy of treatment for an immune-mediated or inflammatory prothrombotic condition to determine whether treatment should continue or cease. Thus also disclosed herein is a method of determining the duration of treatment of an immune-mediated or inflammatory prothrombotic condition in a subject, which comprises determining whether the subject exhibits an increased proportion of procoagulant platelets, wherein an increased proportion of procoagulant platelets in the test sample compared to the at least one control sample indicates that the subject exhibits an increased proportion of procoagulant platelets, determined in accordance with the method described in sections 1 to 1.5 above, and continuing to administer treatment of the immune-mediated or inflammatory prothrombotic condition only if the subject exhibits an increased proportion of procoagulant platelets, or ceasing administration of treatment of the immune-mediated or inflammatory prothrombotic condition if the subject does not exhibit an increased proportion of procoagulant platelets.

Also disclosed herein is a method of reducing side effects associated with a treatment of an immune-mediated or inflammatory prothrombotic condition in a subject, which comprises determining if the subject is at increased risk of experiencing a thrombotic event in accordance with the method of diagnosis described in sections 1 to 1.5 above, and administering or escalating treatment for the immune-mediated or inflammatory prothrombotic condition, only if the subject is determined to be at increased risk of suffering a thrombotic event, or not administering or not escalating the treatment for the immune-mediated or inflammatory prothrombotic condition to the subject, if the subject is not at risk of experiencing a thrombotic event.

2.1 Treatment Etc. Of HIT

In one aspect of the invention, there is provided a method of treatment of HIT in a subject, the method comprising:
a. Determining if the subject is suffering from HIT, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from HIT, wherein an increased proportion of procoagulant platelets in the test sample compared to the at least one control sample indicates that the subject is suffering from HIT,
b. Administering a treatment for HIT to the subject, only if the subject is determined to be suffering from HIT.

In one aspect of the invention, there is provided a method of treatment of HIT in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for HIT, only if the subject is at risk of experiencing a thrombotic event.

In one aspect of the invention, there is provided a method of selecting a therapy for treatment of HIT in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of HIT if the proportion of procoagulant platelets in the therapy test sample is lower than the proportion of procoagulant platelets in the test sample.

In one aspect of the invention, there is provided a method of determining the duration of treatment of HIT in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of HIT only if the subject exhibits an increased proportion of procoagulant platelets, or
c. Ceasing administration of treatment for HIT if the subject does not exhibit an increased proportion of procoagulant platelets.

In one aspect of the invention, there is provided a method of reducing side effects associated with a treatment of HIT in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for HIT to the subject, only if the subject is at risk of experiencing a thrombotic event, or
c. Not administering or not escalating the treatment for HIT to the subject, if the subject is not at risk of experiencing a thrombotic event.

Wherein the immune-mediated or inflammatory prothrombotic condition is HIT, and heparin has been administered to the subject, it is important to cease heparin administration and commence an alternative non-heparin anticoagulant. However, alternative anticoagulants are associated with an increased risk of bleeding complications in the subject and should be avoided if possible. It would therefore be useful to be able to distinguish between thrombocytopenia induced by heparin, and non-heparin induced thrombocytopenia.

Thus a method of treatment of HIT comprises determining if the subject is suffering from HIT in accordance with the method of diagnosis described above in sections 1 and 1.1, and ceasing heparin administration and commencing a non-heparin anticoagulant, only if the subject is determined to be suffering from HIT. Administration of heparin is continued only if the subject is determined to not be suffering from HIT.

Where the subject is commenced on a non-heparin anticoagulant, the non-heparin anticoagulant may be selected from the group consisting of argatroban, danaparoid, and fondaparinux, direct FXa inhibitors (such as rivaroxaban) and direct thrombin inhibitors.

2.2 Treatment Etc. Of VITT

In one aspect of the invention, there is provided a method of treatment of VITT in a subject, the method comprising:
a. Determining if the subject is suffering from VITT, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from VITT, wherein an increased proportion of procoagulant platelets in the test sample compared to the at least one control sample indicates that the subject is suffering from VITT,
b. Administering a treatment for VITT to the subject, only if the subject is determined to be suffering from VITT.

In one aspect of the invention, there is provided a method of treatment of VITT in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for VITT, only if the subject is at risk of experiencing a thrombotic event.

In one aspect of the invention, there is provided a method of determining the duration of treatment of VITT in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of VITT only if the subject exhibits an increased proportion of procoagulant platelets, or
c. Ceasing administration of treatment for VITT if the subject does not exhibit an increased proportion of procoagulant platelets.

In one aspect of the invention, there is provided a method of reducing side effects associated with a treatment of VITT in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for VITT to the subject, only if the subject is at risk of experiencing a thrombotic event, or
c. Not administering or not escalating the treatment for VITT to the subject, if the subject is not at risk of experiencing a thrombotic event.

Where a subject is suffering from VITT, and heparin has been administered to the subject, it is important to cease heparin administration and commence an alternative non-heparin anticoagulant. However, alternative anticoagulants are associated with an increased risk of bleeding complications in the subject and should be avoided if possible. It would therefore be useful to be able to distinguish between thrombocytopenia induced by VITT, and thrombocytopenia due to other causes.

Recommended treatments for VITT include immediate commencement of immune modulating therapy, including commencement of immunoglobulin therapy (IVIg). Immunosuppression (such as steroids, and chemotherapy, such as cyclophosphamide) or plasma exchange, can also be considered. Platelet transfusions are avoided. It is important to be able to identify when such treatment is required, but also when such treatment is not required, as the treatment may result in dangerous side effects such as increased bleeding and infection rates.

Thus a method of treatment of VITT comprises determining if the subject is suffering from VITT in accordance with the method of diagnosis described in sections 1 and 1.2 above, and if the subject is determined to be suffering from VITT, ceasing the administration of heparin if the subject has been administered heparin, and commencing administration of a non-heparin anticoagulant; commencement of immunoglobulin therapy (IVIg); and/or commencement of immunosuppressive therapy; and/or commencement of plasma exchange. Administration of heparin and other standard anti-thrombotic therapy is continued only if the subject is determined to not be suffering from VITT.

Where the subject is commenced on a non-heparin anticoagulant, the non-heparin anticoagulant may be selected from the group consisting of argatroban, danaparoid, and fondaparinux, direct FXa inhibitors (such as rivaroxaban) and direct thrombin inhibitors.

### 2.2.1 Selection of Therapy for VITT

In one aspect of the invention, there is provided a method of selecting a therapy for treatment of VITT in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of VITT if the proportion of procoagulant platelets in the therapy test sample is lower than the proportion of procoagulant platelets in the test sample.

In one embodiment there is provided a method of selecting a therapy for treatment of VITT in a subject, which comprises determining if the therapy causes a reduction in the proportion of procoagulant platelets in the subject. Whether the therapy causes a reduction in the proportion of procoagulant platelets in the subject is determined by comparing the proportion of procoagulant platelets in the test sample and in a therapy test sample, determined in accordance with the method described in sections 1 and 1.2 above, and selecting the therapy for treatment of VITT if the proportion of procoagulant platelets in the therapy test sample is lower than the proportion of procoagulant platelets in the test sample.

In this embodiment, the therapy may be selected from the group consisting of plasma exchange, non-heparin anticoagulants and immunoglobulin (IVIg). The anticoagulant may be fondaparinux.

The therapy test sample is obtained by contacting the test sample with the therapy. The concentration of the therapy is selected to reflect the therapeutic concentration. For example, where the therapy is fondaparinux, the therapy test sample may contain about 0.5-2 μg/mL of fondaparinux, or about 0.5, 1, 1.2, 1.5, or 2 μg/mL of fondaparinux.

In the method of selecting a therapy for treatment of VITT, the proportion of procoagulant platelets in the therapy test sample is lower than the proportion of procoagulant platelets in the test sample means that the proportion of procoagulant platelets in the therapy test sample is reduced to at least 0.99-fold compared to the proportion of procoagulant platelets in the therapy test sample is reduced to at least 0.95, 0.9, 0.8, 0.7, 0.6, 0.5, 0.4, 0.3, 0.2, 0.1, or 0.05-fold of the proportion of procoagulant platelets in the test sample. Where the proportion of procoagulant platelets in the therapy test sample is lower than the proportion of procoagulant platelets in the test sample this is understood to mean that the therapy is an effective therapy for the subject.

The method of selecting a therapy for treatment of VITT can further comprise contacting the test sample with varying concentrations of the therapy to obtain multiple therapy test samples, such that if a concentration-dependent reduction in the procoagulant platelets is observed, this can provide further confirmation of the efficacy of the therapy.

### 2.3 Treatment of Antiphospholipid Syndrome (APS)

In one aspect of the invention, there is provided a method of treatment of APS in a subject, the method comprising:
a. Determining if the subject is suffering from APS, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from APS, wherein an increased proportion of procoagulant platelets in the test sample compared to the at least one control sample indicates that the subject is suffering from APS,
b. Administering a treatment for APS to the subject, only if the subject is determined to be suffering from APS.

In one aspect of the invention, there is provided a method of treatment of APS in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for APS, only if the subject is at risk of experiencing a thrombotic event.

In one aspect of the invention, there is provided a method of selecting a therapy for treatment of APS in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of APS if the proportion of procoagulant platelets in the therapy test sample is lower than the proportion of procoagulant platelets in the test sample.

In one aspect of the invention, there is provided a method of determining the duration of treatment of APS in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of APS only if the subject exhibits an increased proportion of procoagulant platelets, or
c. Ceasing administration of treatment for APS if the subject does not exhibit an increased proportion of procoagulant platelets.

In one aspect of the invention, there is provided a method of reducing side effects associated with a treatment of APS in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for APS to the subject, only if the subject is at risk of experiencing a thrombotic event, or
c. Not administering or not escalating the treatment for APS to the subject, if the subject is not at risk of experiencing a thrombotic event.

Subjects suffering from APS may be generally symptom free, but periodically, or in certain circumstances, be at increased risk of suffering a thrombotic event. When a subject suffering from anti-phospholipid syndrome is at increased risk of suffering a thrombotic event, they require treatment for APS, such as anticoagulation treatment, anti-platelet agents, intravenous immunoglobulin, or hydroxychloroquine, and on occasion, immunosuppression (such as steroids and chemotherapy such as cyclophosphamide) or plasma exchange, to be administered or escalated. It is important to be able to identify when such treatment is required, but also when such treatment is not required, as the treatment may result in dangerous side effects such as increased bleeding and infection rates.

In one embodiment, there is provided a method of treating APS in a subject, the method comprising determining whether the subject is suffering from APS using the method of diagnosis described above in sections 1 and 1.3, and administering treatment for APS, only if the subject is determined to be suffering from APS.

In one embodiment, there is provided a method of reducing the side effects associated with administration of a treatment for APS in a subject, the method comprising determining if the subject is at risk of experiencing a thrombotic event using the method of diagnosis described above in sections 1 and 1.3. The method further comprises administering or escalating treatment for APS, only if the subject is at risk of experiencing a thrombotic event, or not administering or escalating treatment for APS, only if the subject is not at risk of experiencing a thrombotic event.

2.4 Treatment Etc. Of Viral Pneumonia e.g. COVID-19

In one aspect of the invention, there is provided a method of treatment of viral pneumonia in a subject, the method comprising:
a. Determining if the subject is suffering from viral pneumonia, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from viral pneumonia, wherein an increased proportion of procoagulant platelets in the test sample compared to the at least one control sample indicates that the subject is suffering from viral pneumonia,
b. Administering a treatment for viral pneumonia to the subject, only if the subject is determined to be suffering from viral pneumonia.

In one aspect of the invention, there is provided a method of treatment of viral pneumonia in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for viral pneumonia, only if the subject is at risk of experiencing a thrombotic event.

In one aspect of the invention, there is provided a method of selecting a therapy for treatment of viral pneumonia in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of viral pneumonia if the proportion of procoagulant platelets in the therapy test sample is lower than the proportion of procoagulant platelets in the test sample.

In one aspect of the invention, there is provided a method of determining the duration of treatment of viral pneumonia in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of viral pneumonia only if the subject exhibits an increased proportion of procoagulant platelets, or
c. Ceasing administration of treatment for viral pneumonia if the subject does not exhibit an increased proportion of procoagulant platelets.

In one aspect of the invention, there is provided a method of reducing side effects associated with a treatment of viral pneumonia in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for viral pneumonia to the subject, only if the subject is at risk of experiencing a thrombotic event, or
c. Not administering or not escalating the treatment for viral pneumonia to the subject, if the subject is not at risk of experiencing a thrombotic event.

Subjects suffering from viral pneumonia, e.g. COVID-19, are variably at risk of suffering a thrombotic event. When a subject suffering from viral pneumonia e.g. COVID-19 is at increased risk of suffering a thrombotic event, they are not protected by standard preventative (prophylactic) doses of anticoagulant and may require escalation to treatment or intermediate dose anticoagulation treatment. It is important to be able to identify when such treatment is required, but also when such treatment is not required, as the treatment may result in dangerous side effects such as increased bleeding.

Thus a method of treatment of viral pneumonia comprises determining if a subject suffering from viral pneumonia is at increased risk of suffering a thrombotic event in accordance with the method of diagnosis described above in sections 1 and 1.4, and escalating anticoagulant treatment, only if the subject is determined to be at increased risk of suffering a thrombotic event.

### Treatment of Sepsis

In one aspect of the invention, there is provided a method of treatment of sepsis in a subject, the method comprising:
a. Determining if the subject is suffering from sepsis, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from sepsis, wherein an increased proportion of procoagulant platelets in the test sample compared to the at least one control sample indicates that the subject is suffering from sepsis,
b. Administering a treatment for sepsis to the subject, only if the subject is determined to be suffering from sepsis.

In one aspect of the invention, there is provided a method of treatment of sepsis in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for sepsis, only if the subject is at risk of experiencing a thrombotic event.

In one aspect of the invention, there is provided a method of selecting a therapy for treatment of sepsis in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of sepsis if the proportion of procoagulant platelets in the therapy test sample is lower than the proportion of procoagulant platelets in the test sample.

In one aspect of the invention, there is provided a method of determining the duration of treatment of sepsis in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of sepsis only if the subject exhibits an increased proportion of procoagulant platelets, or
c. Ceasing administration of treatment for sepsis if the subject does not exhibit an increased proportion of procoagulant platelets.

In one aspect of the invention, there is provided a method of reducing side effects associated with a treatment of sepsis in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for sepsis to the subject, only if the subject is at risk of experiencing a thrombotic event, or
c. Not administering or not escalating the treatment for sepsis to the subject, if the subject is not at risk of experiencing a thrombotic event.

Subjects suffering from sepsis are variably at risk of suffering a thrombotic event. When a subject suffering from sepsis is at increased risk of suffering a thrombotic event, they may require escalation to treatment or intermediate dose anticoagulation treatment. It is important to be able to identify when such treatment is required, but also when such treatment is not required, as the treatment may result in dangerous side effects such as increased bleeding.

Thus a method of treatment of sepsis comprises determining if a subject suffering from sepsis is at increased risk of suffering a thrombotic event in accordance with the method of diagnosis described above in sections 1 and 1.5, and escalating anticoagulant treatment, only if the subject is determined to be at increased risk of suffering a thrombotic event.

The invention may be described according to the following numbered embodiments.

1. A method of diagnosing an immune-mediated or inflammatory prothrombotic condition in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
   i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition.

2. The method of form 1, wherein the assay control sample is obtained by contacting said test sample or said at least one control sample with GSCA and a control for the alpha granule detection agent.

3. The method of form 1 or form 2, wherein the alpha granule detection agent is an antibody and the control for the alpha granule detection agent is an antibody isotype control.

4. The method of any one of forms 1 to 4, wherein the increased proportion of procoagulant platelets in the test sample compared to the at least one control sample which indicates that the subject is suffering from the immune-mediated or inflammatory prothrombotic condition is an increase of at least about 1.1-fold.

5. The method of any one of forms 1 to 4, wherein determining the uptake of GSAO and surface expression of alpha granule protein of platelets in step c.i. is carried out by flow cytometry.

6. The method of any one of forms 1 to 5, wherein the alpha granule detection agent is selected from the group consisting of an anti-P-selectin antibody, and an anti-TLT-1 antibody.

7. The method of any one of forms 1 to 6, wherein the alpha granule detection agent is an anti-P-selectin antibody.

8. The method of any one of forms 1 to 7, wherein the healthy donor is a FcγRIIa high responder.

9. The method of any one of forms 1 to 8 wherein the test sample contains less than 100 μL of whole blood obtained from the healthy donor.

10. The method of any one of forms 1 to 9, wherein the test sample does not contain exogeneous PF4.

11. The method of any one of forms 1 to 10, wherein the at least one agonist is selected from the group consisting of CRP-xL, SFLLRN, ADP, thrombin, and combinations thereof.

12. The method of any one of forms 1 to 11, wherein the immune-mediated or inflammatory prothrombotic condition is selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers.

13. The method of any one of forms 1 to 12, wherein the at least one control sample comprises a healthy control sample, which comprises healthy donor whole blood contacted with the agonist, and does not comprise subject plasma.

14. The method of form 13, wherein the agonist is a combination of CRP-xL and thrombin.

15. The method of form 13 or form 14, wherein the increased proportion of procoagulant platelets in the test sample compared to the healthy control sample which indicates that the subject is suffering from the prothrombotic condition is an increase of at least about 1.1-fold.

16. The method of any one of forms 1 to 12, wherein the immune-mediated or inflammatory prothrombotic condition is heparin-induced thrombocytopenia (HIT).

17. The method of form 16, wherein step b. further comprises contacting the test sample with therapeutic concentration heparin.

18. The method of form 16 or form 17, wherein the at least one control sample comprises a no heparin control sample and a high heparin control sample, wherein the no heparin control sample comprises the combined sample, the agonist, and no heparin, and wherein the high heparin control sample comprises the combined sample, the agonist, and high concentration heparin.

19. The method of form 17 or form 18, wherein the therapeutic concentration of heparin is between about 0.1 and about 0.7 U/mL.

20. The method of form 18 or form 19, wherein the high concentration of heparin is about 50 to 200 U/mL.

21. The method of any one of forms 16 to 20, wherein the at least one agonist is selected from the group consisting of CRP-xL, SFLLRN, ADP, and combinations thereof.

22. The method of any one of forms 16 to 21, wherein the at least one agonist is SFLLRN.

23. The method of any one of forms 17 to 22, wherein the at least one agonist is SFLLRN and the proportion of procoagulant platelets in the test sample contacted with therapeutic concentration heparin compared to the no heparin control sample which indicates that the subject is suffering from HIT is an increase of between about 2 and 10-fold, and the proportion of procoagulant platelets in the test sample contacted with therapeutic concentration heparin compared to the high heparin control sample which indicates that the subject is suffering from HIT is an increase of between about 2 and 20-fold.

24. The method of any one of forms 16 to 23, wherein the method is capable of differentiating a subject suffering from HIT from a subject suffering from acute thrombocytopenia which is not heparin-induced.

25. The method of any one of forms 1 to 12, wherein the immune-mediated or inflammatory prothrombotic condition is vaccine-induced immune thrombotic thrombocytopenia (VITT).

26. The method of form 25, wherein the subject has been administered a vaccine.

27. The method of form 26, wherein the vaccine is a recombinant adenoviral vector vaccine.

28. The method of form 26 or form 27, wherein the vaccine is a recombinant adenoviral vector encoding the spike protein antigen of SARS-CoV-2.

29. The method of any one of forms 26 to 28, wherein the vaccine is selected from the AstraZeneca AZD1222 (Vaxzevria) vaccine, the Johnson & Johnson/Janssen JNJ-78436735 vaccine, the CanSino Biologics AD5-nCOV vaccine, and the Gamaleya Research Institute of Epidemiology and Microbiology Gam-COVID-Vac vaccine.

30. The method of any one of forms 25 to 29, wherein the at least one agonist is selected from the group consisting of SFLLRN and thrombin.

31. The method of any one of forms 25 to 30, wherein the at least one agonist is SFLLRN.

32. The method of form 31, wherein step b. further comprises contacting the test sample with therapeutic concentration heparin to obtain a therapeutic heparin sample, and further comprises contacting the test sample with high concentration heparin to obtain a high heparin sample, and wherein step d. further comprises determining the proportion of procoagulant platelets in the therapeutic heparin sample and the high heparin sample.

33. The method of form 31 or form 32, wherein the at least one control sample is a healthy control sample, comprising healthy donor whole blood contacted with the at least one agonist, and does not comprise subject plasma.

34. The method of form 32 or form 33, wherein the therapeutic concentration of heparin is between about 0.1 and about 0.7 U/mL.

35. The method of any one of forms 32 to 34, wherein the high concentration of heparin is about 50 to 200 U/mL.

36. The method of any one of forms 32 to 35, wherein, compared to the healthy control sample:
(i) an increase in the proportion of procoagulant platelets in the test sample of greater than 2-fold, a change in the proportion of procoagulant platelets in the therapeutic heparin sample of up to 1.5-fold, and a change in the proportion of procoagulant platelets in the high heparin sample of up to 1.5-fold, indicates that the subject is suffering from classical VITT;
(ii) an increase in the proportion of procoagulant platelets in the test sample of greater than 1-fold, an increase in the proportion of procoagulant platelets in the therapeutic heparin sample of greater than 2-fold, and a change in the proportion of procoagulant platelets in the high heparin sample of up to 1.5-fold, indicates that the subject is suffering from heparin-enhancing VITT; and
(iii) a change in the proportion of procoagulant platelets in the test sample of up to 1.5-fold, a change in the proportion of procoagulant platelets in the therapeutic heparin sample of up to 1.5-fold, and a change in the proportion of procoagulant platelets in the high heparin sample of up to 1.5-fold, indicates that the subject is not suffering from VITT,
wherein the healthy control sample comprises healthy donor whole blood contacted with the at least one agonist, and does not comprise subject plasma.

37. The method of form 36, wherein the test sample is further contacted with the monoclonal antibody IV.3.

38. The method of form 37, wherein, compared to the test sample:
(iv) a decrease in the proportion of procoagulant platelets in the test sample further contacted with the monoclonal antibody IV.3 to 0.7-fold or greater indicates that the subject is suffering from classical VITT or heparin-enhancing VITT.

39. The method of any one of forms 25 to 30, wherein the at least one agonist is thrombin.

40. The method of form 39, wherein the at least one control sample is a healthy control sample, comprising healthy donor whole blood contacted with the at least one agonist, and does not comprise subject plasma.

41. The method of form 40, wherein the increased proportion of procoagulant platelets in the test sample compared to the healthy control sample which indicates that the subject is suffering from VITT is an increase of at least about 2-fold.

42. The method of any one of forms 25 to 41, wherein the method is capable of differentiating a subject suffering from VITT from a subject suffering from acute thrombocytopenia which is not vaccine-induced.

43. The method of any one of forms 1 to 12, wherein the prothrombotic condition is antiphospholipid syndrome.

44. The method of form 43, wherein the at least one control sample comprises a healthy control sample, which comprises healthy donor whole blood contacted with the agonist, and does not comprise subject plasma.

45. The method of form 43 or form 44, wherein the agonist is thrombin.

46. The method of form 44 or form 45, wherein the increased proportion of procoagulant platelets in the test sample compared to the healthy control sample which indicates that the subject is suffering from the prothrombotic condition is an increase of at least about 1.1-fold.

47. A method of treatment of an immune-mediated or inflammatory prothrombotic condition in a subject, the method comprising:
a. Determining if the subject is suffering from the immune-mediated or inflammatory prothrombotic condition, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition,
b. Administering a treatment for the immune-mediated or inflammatory prothrombotic condition to the subject, only if the subject is determined to be suffering from the immune-mediated or inflammatory prothrombotic condition.

48. A method of treatment of an immune-mediated or inflammatory prothrombotic condition in a subject, the method comprising:
a. Determining if the subject if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating treatment for the immune-mediated or inflammatory prothrombotic condition, only if the subject is at risk of experiencing a thrombotic event.

49. A method of selecting a therapy for treatment of an immune-mediated or inflammatory prothrombotic condition in a subject, the method comprising:
a. Determining if the therapy causes a reduction in the proportion of procoagulant platelets in the subject, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Contacting the test sample with the therapy to obtain a therapy test sample,
   iii. Determining the proportion of procoagulant platelets in the test sample and in the therapy test sample, said determining comprising:
      1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of the immune-mediated or inflammatory prothrombotic condition if the proportion of procoagulant platelets in the therapy test sample is lower than the proportion of procoagulant platelets in the test sample.

50. A method of determining the duration of treatment of an immune-mediated or inflammatory prothrombotic condition in a subject, the method comprising:
a. Determining if the subject exhibits an increased proportion of procoagulant platelets, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject exhibits an increased proportion of procoagulant platelets, wherein an increased proportion of procoagulant platelets in the test sample compared to the at least one control sample indicates that the subject exhibits an increased proportion of procoagulant platelets,
b. Continuing to administer treatment of the immune-mediated or inflammatory prothrombotic condition only if the subject exhibits an increased proportion of procoagulant platelets, or
c. Ceasing administration of treatment of the immune-mediated or inflammatory prothrombotic condition if the subject does not exhibit an increased proportion of procoagulant platelets.

51. A method of reducing side effects associated with a treatment of an immune-mediated or inflammatory prothrombotic condition in a subject, the method comprising:
a. Determining if the subject is at risk of experiencing a thrombotic event, said determining comprising:
   i. Combining a sample of plasma obtained from the subject with a sample of whole blood obtained from a healthy donor to obtain a combined sample,
   ii. Contacting the combined sample with at least one agonist to obtain a test sample,
   iii. Determining the proportion of procoagulant platelets in the test sample and in at least one control sample, said determining comprising:
      1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
   iv. Determining whether the subject is at risk of experiencing a thrombotic event, wherein an increased proportion of procoagulant platelets in the test sample compared to the at least one control sample indicates that the subject is at risk of experiencing a thrombotic event,
b. Administering or escalating the treatment for the immune-mediated or inflammatory prothrombotic condition to the subject, only if the subject is at risk of experiencing a thrombotic event, or
c. Not administering or not escalating the treatment for the immune-mediated or inflammatory prothrombotic condition to the subject, if the subject is not at risk of experiencing a thrombotic event.

52. The method of any one of forms 47 to 51, wherein the assay control sample is obtained by contacting said test sample or said at least one control sample with GSCA and a control for the alpha granule detection agent.

53. The method of any one of forms 47 to 52, wherein the alpha granule detection agent is an antibody and the control for the alpha granule detection agent is an antibody isotype control.

54. The method of any one of forms 47 to 53, wherein the increased proportion of procoagulant platelets in the test sample compared to the at least one control sample which indicates that the subject is suffering from the prothrombotic condition is an increase of at least about 1.1-fold.

55. The method of any one of forms 47 to 54, wherein determining the uptake of GSAO and surface expression of alpha granule protein of platelets in step c.i. is carried out by flow cytometry.

56. The method of any one of forms 47 to 55, wherein the alpha granule detection agent is selected from the group consisting of an anti-P-selectin antibody, and an anti-TLT-1 antibody.

57. The method of any one of forms 47 to 56, wherein the alpha granule detection agent is an anti-P-selectin antibody.

58. The method of any one of forms 47 to 57, wherein the healthy donor is a FcγRIIa high responder.

59. The method of any one of forms 47 to 58 wherein the test sample contains less than 100 μL of whole blood obtained from the healthy donor.

60. The method of any one of forms 47 to 59, wherein the test sample does not contain exogeneous PF4.

61. The method of any one of forms 47 to 60, wherein the at least one agonist is selected from the group consisting of CRP-xL, SFLLRN, ADP, thrombin, TRAP, and combinations thereof.

62. The method of any one of forms 47 to 61, wherein the immune-mediated or inflammatory prothrombotic condition is selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers.

63. The method of form 62, wherein the at least one control sample comprises a healthy control sample, which comprises healthy donor whole blood contacted with the agonist, and does not comprise subject plasma.

64. The method of form 62 or form 63, wherein the agonist is a combination of CRP-xL and thrombin.

65. The method of form 63 or form 64, wherein the increased proportion of procoagulant platelets in the test sample compared to the healthy control sample which indicates that the subject is suffering from the prothrombotic condition is an increase of at least about 1.1-fold.

66. The method of any one of forms 47 to 62, wherein heparin has been administered to the subject and the immune-mediated or inflammatory prothrombotic condition is heparin-induced thrombocytopenia (HIT).

67. The method of form 66, wherein step a.ii. further comprises contacting the test sample with therapeutic concentration heparin.

68. The method of form 66 or form 67, wherein the at least one control sample comprises a no heparin control sample and a high heparin control sample, wherein the no heparin control sample comprises the combined sample, the agonist, and no heparin, and wherein the high heparin control sample comprises the combined sample, the agonist, and high concentration heparin.

69. The method of form 67 or form 68, wherein the therapeutic concentration of heparin is between about 0.1 and about 0.7 U/mL.

70. The method of form 68 or form 69, wherein the high concentration of heparin is about 50 to 200 U/mL.

71. The method of any one of forms 66 to 70, wherein the at least one agonist is selected from the group consisting of CRP-xL, SFLLRN, ADP, and combinations thereof.

72. The method of any one of forms 66 to 71, wherein the at least one agonist is SFLLRN.

73. The method of any one of forms 67 to 72, wherein the at least one agonist is SFLLRN and the proportion of procoagulant platelets in the test sample contacted with therapeutic concentration heparin compared to the no heparin control sample which indicates that the subject is suffering from HIT is an increase of between about 2 and 10-fold, and the proportion of procoagulant platelets in the test sample contacted with therapeutic concentration heparin compared to the high heparin control sample which indicates that the subject is suffering from HIT is an increase of between about 2 and 20-fold.

74. The method of any one of forms 67 to 73, wherein administering a treatment for HIT comprises ceasing the administration of heparin and commencing administration of a non-heparin anticoagulant.

75. The method of any one of forms 66 to 74, further comprising:
c. continuing the administration of heparin, only if the subject is determined to not be suffering from HIT.

76. The method of any one of forms 47 to 62, wherein the immune-mediated or inflammatory prothrombotic condition is vaccine-induced immune thrombotic thrombocytopenia (VITT).

77. The method of any one of form 76, wherein the subject has been administered a vaccine.

78. The method of form 77, wherein the vaccine is a recombinant adenoviral vector vaccine.

79. The method of form 77 or form 78, wherein the vaccine is a recombinant adenoviral vector encoding the spike protein antigen of SARS-CoV-2.

80. The method of any one of forms 77 to 79, wherein the vaccine is selected from the AstraZeneca AZD1222 (Vaxzevria) vaccine, the Johnson & Johnson/Janssen JNJ-78436735 vaccine, the CanSino Biologics AD5-nCOV vaccine, and the Gamaleya Research Institute of Epidemiology and Microbiology Gam-COVID-Vac vaccine.

81. The method of any one of forms 76 to 80, wherein the at least one agonist is selected from the group consisting of SFLLRN and thrombin.

82. The method of any one of forms 76 to 81, wherein the at least one agonist is SFLLRN.

83. The method of form 82, wherein step b. further comprises contacting the test sample with therapeutic concentration heparin to obtain a therapeutic heparin sample, and further comprises contacting the test sample with high concentration heparin to obtain a high heparin sample, and wherein step d. further comprises determining the proportion of procoagulant platelets in the therapeutic heparin sample and the high heparin sample.

84. The method of form 82 or form 83, wherein the at least one control sample is a healthy control sample, comprising healthy donor whole blood contacted with the at least one agonist, and does not comprise subject plasma.

85. The method of form 83 or form 84, wherein the therapeutic concentration of heparin is between about 0.1 and about 0.7 U/mL.

86. The method of any one of forms 83 to 85, wherein the high concentration of heparin is about 50 to 200 U/mL.

87. The method of any one of forms 83 to 86, wherein, compared to the healthy control sample:
(i) an increase in the proportion of procoagulant platelets in the test sample of greater than 2-fold, a change in the proportion of procoagulant platelets in the therapeutic heparin sample of up to 1.5-fold, and a change in the proportion of procoagulant platelets in the high heparin sample of up to 1.5-fold, indicates that the subject is suffering from classical VITT;
(ii) an increase in the proportion of procoagulant platelets in the test sample of greater than 1-fold, an increase in the proportion of procoagulant platelets in the therapeutic heparin sample of greater than 2-fold, and a change in the proportion of procoagulant platelets in the high heparin sample of up to 1.5-fold, indicates that the subject is suffering from heparin-enhancing VITT;
(iii) a change in the proportion of procoagulant platelets in the test sample of up to 1.5-fold, a change in the proportion of procoagulant platelets in the therapeutic heparin sample of up to 1.5-fold, and a change in the proportion of procoagulant platelets in the high heparin sample of up to 1.5-fold, indicates that the subject is not suffering from VITT, wherein the healthy control sample comprises healthy donor whole blood contacted with the at least one agonist, and does not comprise subject plasma.

88. The method of form 87, wherein the test sample is further contacted with the monoclonal antibody IV.3.

89. The method of form 88, wherein, compared to the test sample:
(iv) a decrease in the proportion of procoagulant platelets in the test sample further contacted with the monoclonal antibody IV.3 to 0.7-fold or greater indicates that the subject is suffering from classical VITT or heparin-enhancing VITT.

90. The method of any one of forms 76 to 81, wherein the at least one agonist is thrombin.

91. The method of form 90, wherein the at least one control sample is a healthy control sample, comprising healthy donor whole blood contacted with the at least one agonist, and does not comprise subject plasma.

92. The method of form 91, wherein the increased proportion of procoagulant platelets in the test sample compared to the healthy control sample which indicates that the subject is suffering from VITT is an increase of at least about 2-fold.

93. The method of any one of forms 76 to 92, wherein administering a treatment for VITT comprises:
(i) ceasing the administration of heparin if the subject has been administered heparin, and commencing administration of a non-heparin anticoagulant; and/or
(ii) commencement of immunoglobulin therapy (IVIg); and/or
(iii) commencement of immunosuppressive therapy; and/or
(iv) commencement of plasma exchange.

94. The method of any one of forms 76 to 93, further comprising:
c. administration of heparin, only if the subject is determined to not be suffering from VITT.

95. The method of form 49, wherein the prothrombotic condition is VITT and the therapy is selected from the group consisting of plasma exchange, non-heparin anticoagulants and immunoglobulin (IVIg).

96. The method of form 95, wherein the non-heparin anticoagulant is fondaparinux.

97. The method of any one of forms 47 to 62, wherein the immune-mediated or inflammatory prothrombotic condition is antiphospholipid syndrome.

98. The method of form 97, wherein the at least one control sample comprises a healthy control sample, which comprises healthy donor whole blood contacted with the agonist, and does not comprise subject plasma.

99. The method of form 97 or form 98, wherein the agonist is thrombin.

100. The method of form 98 or form 99, wherein the increased proportion of procoagulant platelets in the test sample compared to the healthy control sample which indicates that the subject is suffering from the prothrombotic condition is an increase of at least about 1.1-fold.

101. The method of any one of forms 1 to 100 wherein the method is carried out in vitro.

### EXAMPLES

### Heparin-Induced Thrombocytopenia

### Materials and Methods

### Study Approval

Human studies were approved by Concord Repatriation General Hospital (CRGH) Human Research Ethics Committee (HREC/18/CRGH/294), WSLHD Human Research Ethics Committee #1812-01). Blood donors gave written informed consent.

### Materials

GSAO (4-(N—((S-glutathionyl)acetyl)amino)phenylarsonous acid) and control compound GSCA (4-(N—(S-glutathionylacetyl)amino)benzoic acid) were synthesized and conjugated as previously described.⁴ Monomeric collagen-related peptide (CRP, Auspep) was cross-linked using N-succinimidyl 3-(2-pyridyldithio)propionate (Sigma Aldrich) to produce CRP-xL.

### Plasma Samples

Citrated platelet-poor plasma stored at −80° C. were from patients with suspected HIT, referred to diagnostic laboratories in three Australian hospitals (CRGH, Royal Prince Alfred and the ICPMR at Westmead Hospital) for subsequent investigation by SRA at a reference laboratory. SRA was performed following initial positive immunological screening (either AcuStar HIT-IgG_{(PF4-H)} chemiluminescence or STic Expert HIT lateral flow) on patients with intermediate or high pretest probability of HIT based on the 4 Ts scoring system⁴⁻⁸ or by request of treating physicians. SRA-positive samples that fulfilled clinical criteria for HIT were considered HIT+. Clinical notes were adjudicated by independent blinded observers to verify clinical HIT. HIT negative (HIT−) samples were from inpatients with thrombocytopenia or platelet count drop >25%, intermediate/high 4 Ts score with negative immunoassay testing.

### Screening for High Responders

Healthy donors recruited at the CRGH and ANZAC Research Institute (Sydney, Australia) were screened to identify FcγRIIa responders⁸ using light transmission aggregometry as described.⁹

### Blood Collection

Blood was collected from the antecubital fossa of healthy volunteers into 3.2% citrate tubes using a 21 G butterfly needle. The initial 3 mL of blood was discarded.

### Procoagulant Platelet Assay

The GSAO-based whole blood PP flow cytometry assay established by the inventors⁴ was modified to incorporate patient plasma. Citrated whole blood (13 μL) was incubated with and without agonists, unfractionated heparin (Pfizer) and plasma (5 μL) for 10 minutes with final concentrations of 2.5 mM Gly-Pro-Arg-Pro peptide (Sigma-Aldrich) and 2.5 mM calcium chloride. Agonists included CRP-xL, ADP (Helena Laboratories), thrombin receptor-activating peptide (SFLLRN, Auspep), and bovine thrombin (Sigma-Aldrich). In some experiments, blood was pre-treated with 10 μg/mL IV.3 (StemCell Technologies), a monoclonal antibody against FcγRIIa, for 15 minutes prior to the reaction. Samples were incubated with antibodies to CD45, CD41a, CD62P—or isotype control, and GSAO or GSCA, and fixed before analysis on a BD LSRFortessa X-20.

### Clinical Adjudication

Two independent clinicians reviewed electronic medical records to determine HIT diagnosis and thrombotic outcomes, while blinded to assay results.

### Statistical Analyses

Statistical analyses were performed using GraphPad Prism 9, with statistical significance set at P<0.05.

### Results

Plasma from HIT+ Patients Sensitizes Healthy Donor Platelets to Become Procoagulant in the Presence of Strong Platelet Agonists

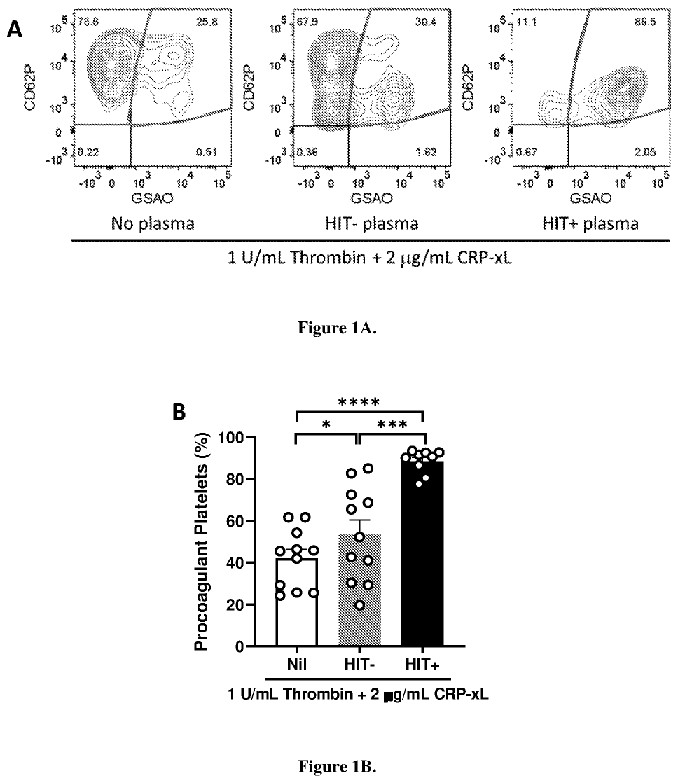

Procoagulant platelets were identified using a whole blood flow cytometry-based assay as CD41a+/CD45− events that were marked with both the cell necrosis marker GSAO-AF647, and the platelet activation marker P-selectin (GSAO+/CD62P+) as previously described.⁴ Pre-incubation with plasma from patients with activating HIT antibodies markedly increased the proportion of procoagulant platelets in the whole blood of healthy donors following combined thrombin and CRP-xL stimulation. Representative flow cytometry plots from a single donor comparing no plasma with HIT− or HIT+ plasma indicate that addition of HIT+ plasma altered the sub-population profile of the platelets with a marked increase in procoagulant (GSAO+/CD62P+) platelets (FIG. 1A). There was a significantly higher proportion of procoagulant platelets in the presence of HIT+ plasma compared with HIT− plasma (88.37±1.90 vs 53.63±6.82, P<0.001) and no plasma (88.37±1.90 vs 42.05±4.24, P<0.0001) (FIG. 1B). There was also a smaller but statistically significant increase seen with HIT− plasma compared to no plasma (53.63±6.82 vs 42.05±4.24, P<0.05) (FIG. 1B).

HIT+ Plasma does not Increase Procoagulant Platelets (PP) with GPCR Agonists Alone or Heparin Alone

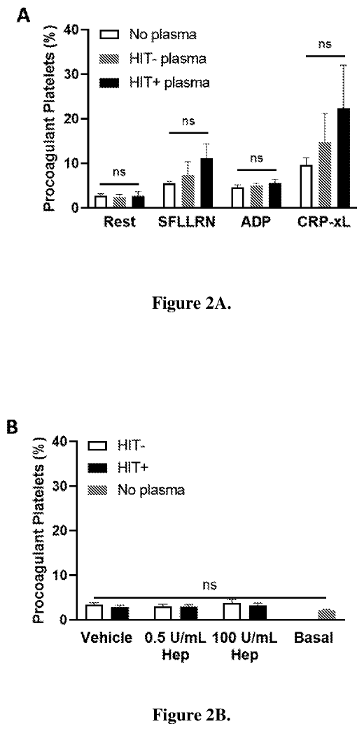

The inventors examined PP response to HIT+ plasma in presence and absence of agonist stimulation of GPCR ligands: PAR-1 agonist (SFLLRN) and P2Y₁₂ receptor agonist (ADP), and ILR ligand GPVI agonist (CRP-xL). In the absence of exogenous heparin, there was no difference in the proportion of PP in the presence of HIT+ plasma compared to either HIT−, no plasma or autologous plasma under resting conditions or following stimulation with SFLLRN, ADP, or CRP-xL (FIG. 2). There were no differences in PP proportion with either HIT+ or HIT− plasma, at either therapeutic-concentration (0.5 U/mL) or high-dose heparin (100 U/mL), compared to basal levels of PP without exogenous plasma (FIG. 2).

HIT+ Plasma Sensitizes Healthy Platelets to Become Procoagulant with GPCR Agonist Stimulation in a Heparin-Dependent Manner and Requires Active FcγRIIa

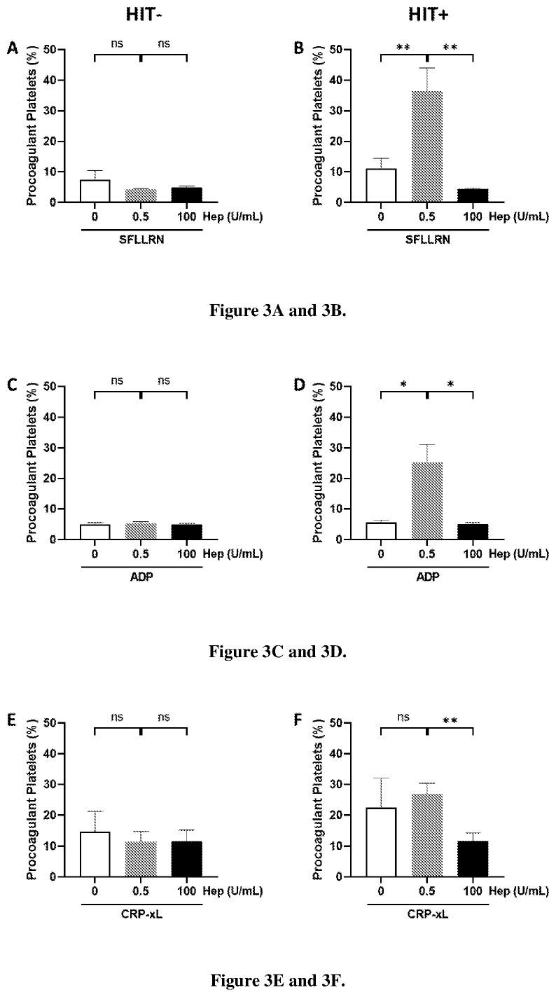

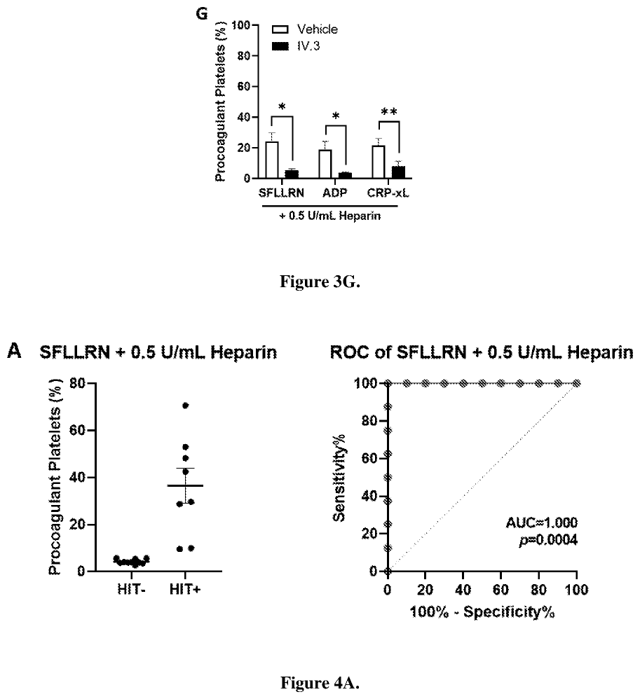

HIT antibodies recognize complexes of heparin with platelet alpha granule protein, PF4, and agonist stimulation is known to release PF4. SFLLRN, ADP or CRP-xL stimulation led to >90% alpha granule release measured by P-selectin. In presence of HIT− plasma, there was no difference in PP formation in response to SFLLRN, ADP or CRP-xL (FIGS. 3A, C and E), at either therapeutic or high heparin concentrations. However, in presence of HIT+ plasma, there was a significant difference in the proportion of PP at therapeutic-concentration heparin compared to no heparin for SFLLRN (36.53±7.50 vs 11.19±3.23, P<0.01) and ADP (25.22±5.94 vs 5.66±0.79, P<0.05) (FIGS. 3B and D). HIT has a well-established stoichiometric ratio of heparin and PF4 underscoring the formation of pathogenic immune complexes,¹⁰ and the heightened PP response at therapeutic-concentration heparin was fully abrogated at high-dose heparin for all agonists tested—SFLLRN (36.53±7.50 vs 4.39±0.35, P<0.01), ADP (25.22±5.94 vs 5.16±0.52, P<0.05) and CRP-xL (26.87±3.58 vs 11.70±2.63, P<0.01) (FIGS. 3B, D and F).

The inventors examined the requirement for FcγRIIa in the sensitization of PP by receptor-specific agonists together with therapeutic-concentration heparin. Pre-treatment of healthy platelets with IV.3 markedly reduced the proportion of PP stimulated with receptor-specific agonists in presence of therapeutic-concentration heparin and HIT+ plasma, with SFLLRN (24.24±5.59 vs 5.63±0.83, P<0.05), ADP (18.98±5.28 vs 3.85±0.34, P<0.05) and CRP-xL (21.63±4.82 vs 8.02±3.24, P<0.01) (FIG. 3G), indicating that active FcγRIIa is required for the HIT plasma-induced and heparin-dependent potentiation of PP formation.

PP Formation Induced by SFLLRN, Therapeutic-Concentration Heparin and Patient Plasma has Potential to Differentiate Thrombocytopenia Secondary to HIT from Other Etiologies

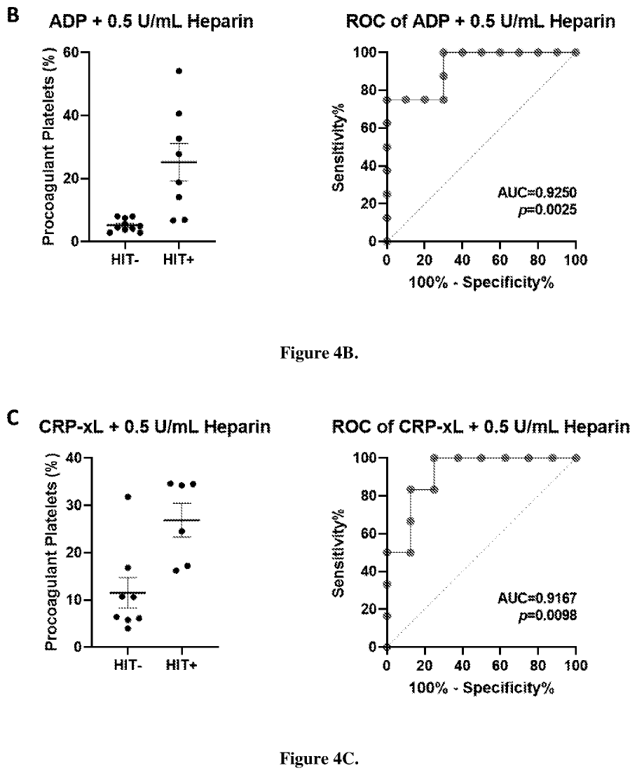

To assess the diagnostic value of a flow cytometry-based PP assay, the inventors compared PP formation using plasma from HIT+ and HIT− patients after SFLLRN stimulation in presence of therapeutic-concentration heparin. This yielded a Receiver Operating Characteristics (ROC) area under the curve (AUC) of 1.0±0, P=0.0004 with a procoagulant result of 7.6% demonstrating 100% sensitivity and specificity (FIG. 4A). ADP under the same conditions yielded a ROC AUC 0.93±0.06, P=0.0025 with a cut-off of 6.2% demonstrating 100% sensitivity and 70% specificity (FIG. 4B), and CRP-xL yielded a ROC AUC of 0.92±0.08, P=0.0098 with a cut-off of 13.5% demonstrating 100% sensitivity and 75% specificity (FIG. 4C).

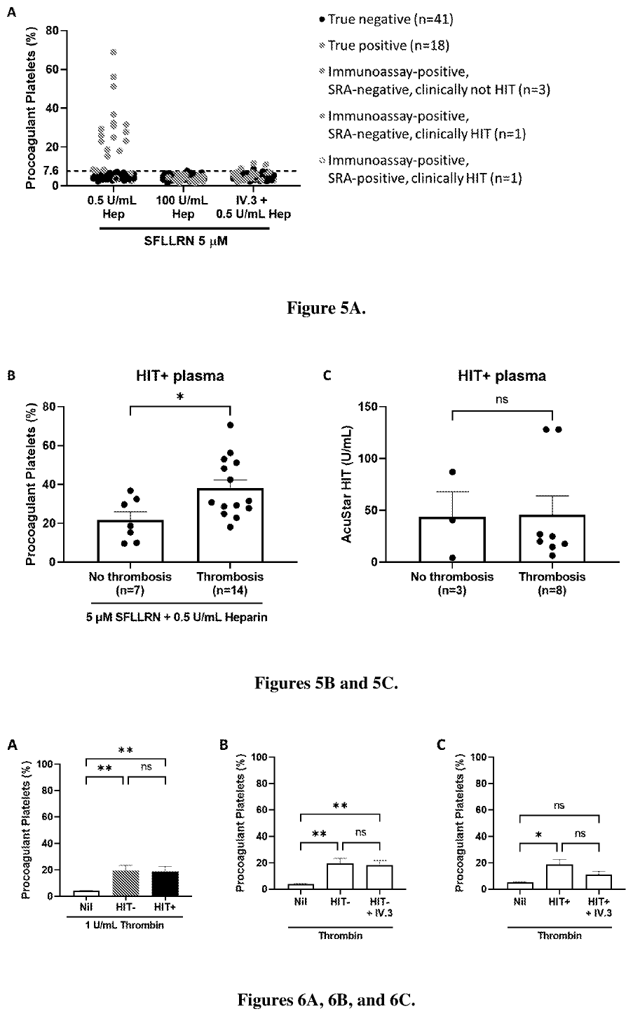

In a validation cohort of 64 patients with clinically suspected HIT, the PP assay with SFLLRN in presence of healthy whole blood, patient plasma and heparin, was performed while blinded to the diagnosis. SRA was again used as the gold standard for HIT+ samples. A PP result of greater than 7.6% correctly identified 43 of 44 immunoassay- or SRA-negative samples and 18 of 20 SRA-positive samples (FIG. 5A). This yielded a positive predictive value of 94.7%, a negative predictive value of 95.6% and an accuracy of 95.3%. Independent blinded clinical review of the HIT diagnosis demonstrated 98% concordance with assay results and final clinical HIT diagnosis. Notably, the assay correctly identified 3 of 3 samples that were false positive by immunoassay, but negative by SRA and negative by clinical review. Two of these were patients with a previous diagnosis of HIT. One of two samples that was SRA-positive but negative in the assay of the invention had a final diagnosis of immune thrombocytopenia while the other was clinically HIT. The assay of the invention correctly identified a false negative SRA sample that was clinically HIT (FIG. 5A). PP response induced by 13 HIT+ plasma did not correlate with anti-PF4/heparin antibody levels by chemiluminescence. Importantly, a higher PP response (P=0.0213, FIG. 5B), was observed in HIT+ patients with thrombotic outcomes compared to those without, whilst anti-PF4/heparin antibody levels by chemiluminescence immunoassay was not associated with thrombosis (P=0.9559, FIG. 5C).

HIT+ Plasma Hypersensitizes Healthy Platelets to Thrombin-Induced PP Response in a Heparin-Independent Manner that is Only Partially Dependent on FcγRIIa

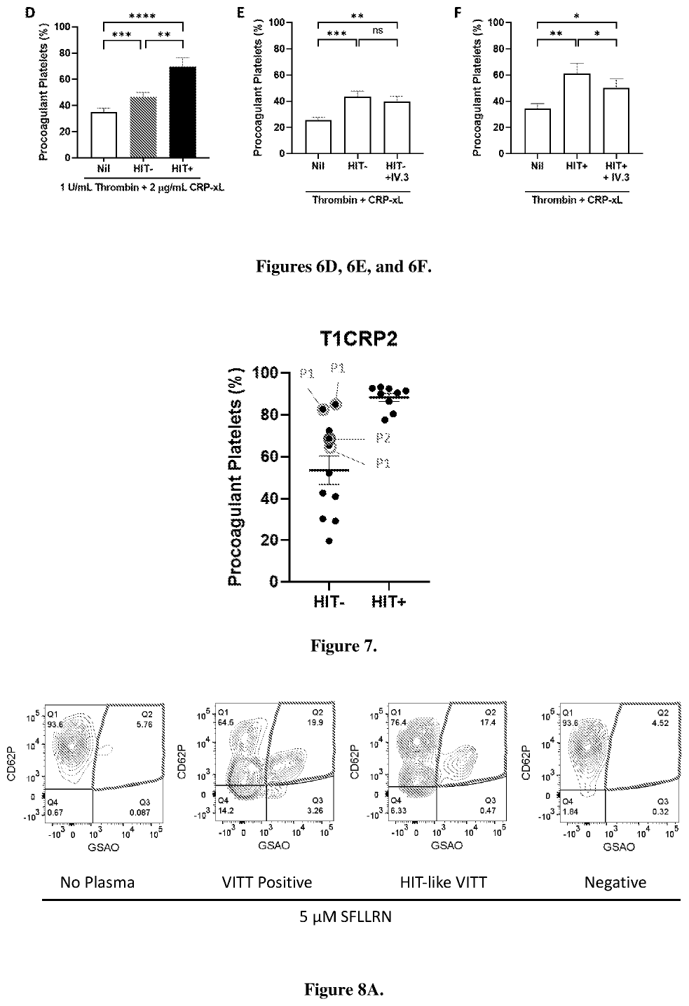

A paradox in HIT is the enhancement of thrombin generation in presence of heparin.¹¹ Thrombosis can occur even after cessation of heparin and initiation of alternative anticoagulation including direct thrombin inhibitors. Hence, the inventors explored the PP response induced by HIT plasma on donor platelets stimulated with thrombin±ILR agonist in the absence of heparin. Pre-incubation with HIT+ or HIT− plasma increased the proportion of PP in healthy donors following thrombin stimulation compared to no plasma (HIT−: 19.55±4.16 vs 4.16±0.24, P=0.0021; HIT+: 18.86±3.94 vs 4.16±0.24, P=0.0039; FIG. 6A). There was no difference in the PP response between HIT+ and HIT− plasma. Blockade of FcγRIIa did not affect the thrombin-induced PP response (FIGS. 6B and C. Combined thrombin+CRP-xL stimulation increased the PP proportion generated by HIT+ plasma (69.57±6.85) compared to no plasma (28.52±2.17, P<0.0001) and HIT− plasma (46.54±3.53, P=0.0020) (FIG. 6D). This hypersensitivity in donor platelets was not observed with autologous plasma (29.45±5.08). A partial inhibition of the PP response by IV.3 was evident with HIT+ (50.16±6.82 vs 60.97±8.04, P=0.0161, FIG. 6F) but not HIT− plasma (39.91±3.95 vs 43.53±4.20, P=0.2259, FIG. 6E), suggesting a partial role for FcγRIIa in the thrombin-driven PP response generated by HIT plasma in the absence of exogenous heparin.

### Procoagulant Platelet Formation in the Presence of T1CRP2 is Increased in Patients Having a Variety of Immune-Mediated or Inflammatory Prothrombotic Conditions

Analysis of the above results identified patients who exhibited an increased proportion of procoagulant platelets, who were suffering from prothrombotic conditions other than HIT (FIG. 7). For example, a patient suffering from anti-phospholipid syndrome demonstrated an increased proportion of procoagulant platelets with T1CRP2 stimulation. A second patient suffering from toxic epidermal necrolysis secondary to chemotherapy also demonstrated an increased proportion of procoagulant platelets with T1CRP2 stimulation. This demonstrates the broader applicability of the method of the invention to prothrombotic conditions in general.

### Analysis of Increases in the Proportion of Procoagulant Platelet in Patient Samples

The average (of different patients combined with different donors), minimum and maximum fold-change in procoagulant platelets observed with HIT+ plasma are as follows. Healthy control samples do not contain healthy donor autologous plasma unless stated otherwise.

TABLE 2

| | Average | | |
|---|---|---|---|
| Conditions | fold-change | Min | Max |
| CRP-xL | | | |
| Combined sample + agonist + therapeutic | 1.9 | 0.5 | 3.1 |
| concentration heparin/no heparin control | | | |
| sample | | | |
| Combined sample + agonist + therapeutic | 2.7 | 1.6 | 4.6 |
| concentration heparin/high heparin control | | | |
| sample | | | |
| SFLLRN | | | |
| Combined sample + agonist + therapeutic | 3.7 | 2.1 | 6.5 |
| concentration heparin/no heparin control | | | |
| sample | | | |
| Combined sample + therapeutic | 8.5 | 1.9 | 16.1 |
| concentration heparin/high heparin control | | | |
| sample | | | |
| ADP | | | |
| Combined sample + agonist + therapeutic | 4.4 | 1.6 | 10.4 |
| concentration heparin/no heparin control | | | |
| sample | | | |
| Combined sample + agonist + therapeutic | 5.5 | 1.2 | 12.6 |
| concentration heparin/high heparin control | | | |
| sample | | | |
| T1CRP2 | | | |
| Combined sample + T1CRP2/healthy control | 2.76 | 1.71 | 5.39 |
| sample | | | |

The average (of different patients combined with different donors), minimum and maximum fold-change in procoagulant platelets observed with plasma from a patient suffering from anti-phospholipid syndrome are as follows.

TABLE 3

| | Average | | |
|---|---|---|---|
| Conditions | fold-change | Min | Max |
| T1CRP2 | | | |
| Combined sample/healthy control sample + | 1.3 | 1.1 | 1.6 |
| autologous plasma | | | |
| Combined sample/healthy control sample | 7.6 | 3.8 | 16.7 |
| Thrombin | | | |
| Combined sample/healthy control sample + | 2.1 | 1.7 | 2.5 |
| autologous plasma | | | |
| Combined sample/healthy control sample | 5.7 | 3.9 | 8.0 |

### Discussion

Early and accurate detection of HIT is critical for improved patient outcomes, yet this remains a clinical challenge. An increased understanding of the mechanisms underlying HIT is helpful for improved diagnostic platforms and therapeutics. PP play an important role in hemostasis by providing a surface for the assembly and propagation of coagulation factors, enabling thrombin generation and subsequent clot stabilization through fibrin formation. A well-known paradox in HIT is that thrombin generation, demonstrable on calibrated thrombography, is enhanced instead of reduced in the presence of heparin.^{11,12} Expanding on the work of Tutwiler and colleagues on coated platelets and HIT,¹ the inventors confirm the marked expansion of the PP surface by HIT+ patient plasma in presence of heparin as a potential mechanism for this paradox. The inventors demonstrate that low-dose GPCR agonist sensitizes platelets to the mechanism of heparin-dependent HIT plasma induction of a PP surface, in a process that is fully dependent on active FcγRIIa. The increase in PP seen at therapeutic-concentration heparin reduced to baseline levels when the stoichiometric ratio between PF4 and heparin was disrupted by high-dose heparin. Furthermore, measurement of SFLLRN-induced PP formation under these conditions can accurately differentiate HIT from non-HIT thrombocytopenia. These results provide important insights into the mechanisms underlying thrombosis in HIT and suggest that our flow cytometry-based PP assay may be a viable strategy for rapid yet accurate diagnosis of HIT.

Using a flow cytometry assay that identifies PP using combined detection of a cell death marker GSAO and platelet activation marker P-selectin to examine the effect of SRA-confirmed HIT+ plasma on the procoagulant profile of healthy donor platelets, the inventors showed that HIT+ plasma-sensitized platelets become markedly procoagulant following agonist stimulation. To gain insight into the pathophysiology of PP in HIT patients, the inventors performed these studies in whole blood rather than washed platelets. In contrast to the classical SRA, whereby low-dose heparin induces serotonin release from washed donor platelets mixed with HIT+ plasma, the inventors found that agonist stimulation was required in this whole blood assay. The inventors speculate that this requirement relates to agonist-induced platelet PF4 release which allows generation of the pathogenic complex without requirement for exogenous PF4. The increase in PP seen at therapeutic-concentration heparin (0.5 U/mL) was completely abrogated when excess heparin offset the required PF4:heparin (or PF4:other polyanion) molar stoichiometry to form a stable HIT immune complex, resulting in the characteristic abrogated response.^{2,10,13,14} Importantly, the sensitizing heparin concentration in the inventors' experiments is within the clinical therapeutic range (0.3-0.7 U/mL),¹⁵ suggesting this heparin-dependent sensitization plausibly contributes to the thrombotic complications seen in patients with HIT in vivo.

Using the KKO and PF4 HIT model, Tutwiler and colleagues previously speculated that the combination of PAR stimulation and ITAM signaling via FcγRIIa was required for annexin V+ “coated” platelet formation in HIT.¹ Here, the increased PP proportion in the presence of HIT plasma and therapeutic-concentration heparin was seen with SFLLRN or ADP stimulation, indicating that while PAR signaling appears to be a potent co-factor, stimulation of alternative GPCR pathways such as P2Y₁₂ also induce PP in presence of HIT antibody-mediated ITAM signaling. This is in agreement with previous findings that ADP and its receptor P2Y₁₂ can induce platelet aggregation with HIT sera,¹⁶ and that P2Y₁₂ blockade inhibits formation of pathological coated platelets and PP.¹⁷⁻²¹ In contrast, sensitization using CRP-xL, an ILR ligand, did not produce a synergistic PP expansion.

The inventors' finding that SFLLRN priming of healthy whole blood allows measurement of heparin-dependent FcγRIIa-mediated PP response, resulted in design of a HIT diagnostic assay with high sensitivity and specificity and a ROC curve of 1.0. Considering the limitations of current diagnostic tests, a new rapid functional assay that maintains the specificity and sensitivity of SRA without use of radioactivity is potentially useful.⁶ A flow cytometry donor platelet, annexin V-based assay for diagnosis of HIT was first proposed by Tomer and colleagues,^{3,22} and PF4-dependent P-selectin expression, was proposed by Padmanabhan and colleagues.²³ Functional platforms like the Multiplate aggregation and immunoassays like the chemiluminescence AcuStar HIT-IgG_{(PF4-H)} offer alternatives.^{6,7,9} The inventors' assay has advantages such as: the use of whole blood rather than washed platelets, minimal volume of plasma required (5 uL per patient), low-dose agonist stimulation rather than PF4, use of a standard diagnostic flow cytometer rather than dedicated platform all increase practicality. Furthermore, higher PP levels in the inventors' assay were seen in HIT patients with thrombosis rather than HIT patients with thrombocytopenia alone, whilst this relationship was not seen with chemiluminescence immunoassay titres.

Importantly, this platform distinguished clinically adjudicated, SRA-confirmed true HIT patients from hospitalized non-HIT, acute thrombocytopenic patients or patients with >25% fall in platelets, referred for HIT testing after 4 Ts screening. HIT− included patients with sepsis, non-heparin drug-induced thrombocytopenia, consumptive thrombocytopenia and anti-phospholipid syndrome. These HIT− thrombocytopenic patients often also have a procoagulant phenotype, but were able to be differentiated by the low- and high-dose heparin conditions and response to IV.3. Plasmas used for the validation cohort were well-characterized, previously used in comparison of chemiluminescence, ELISA and SRA platforms.^{6,7} The high sensitivity and specificity in the development cohort were confirmed in the validation cohort with an overall performance of this assay achieving 98% accuracy when compared to clinical adjudication of final HIT diagnosis. Furthermore, it identified the chemiluminescence immunoassay false positives. This platform could potentially be established in any diagnostic flow cytometry laboratory, shortening time to definitive diagnosis, thus allowing timely cessation of alternative anticoagulants in patients who do not have activating HIT antibodies. Up to 44% of patients eventually demonstrated to be HIT negative, experience major bleeding when commenced on alternative anticoagulation whilst awaiting confirmatory studies.²⁴ This platform has recently been adapted to investigate other FcγRIIa-mediated thrombotic conditions including vaccine-induced immune thrombotic thrombocytopenia.

The inventors' findings provide mechanistic insight into the pathogenesis of HIT. The increased PP response to SFLLRN and heparin from HIT patients with thrombosis compared with HIT with thrombocytopenia alone, suggests that the extent of procoagulant surface may play a pathogenic role.⁴ The inventors' results suggest that in HIT patients treated with therapeutic heparin, small physiological increases in agonist concentrations, plausibly caused by presence of atherosclerotic plaque, stenotic vasculature or surgical trauma, could have a large contribution to PP formation and consequent risk of thrombosis. Indeed, surgical patients are three to four times more likely to develop HIT than medical patients.²⁵⁻²⁸ Targeting FcγRIIa signaling at this stage of HIT may be viable.¹ FcγRIIa does not play a major role in platelet adhesion, thus a therapeutic inhibitor is less likely to cause the increased bleeding seen with non-heparin anticoagulants. The inventors' experiments, performed in whole blood, do not directly investigate the contribution of platelet FcγRIIa signaling compared with signaling via leukocytes. It is therefore possible that the PP expansion observed here is augmented by leukocyte FcγRIIa signaling since platelet transactivation by monocytes is implicated in thrombotic complications in HIT.¹ A humanized version of IV.3, VIB9600, has been developed and assessed in preclinical studies for treatment of immune-mediated proinflammatory conditions like sepsis,²⁹ and appears to have an acceptable safety profile in primates. Early and direct targeting of the pathogenic pathway of HIT to abrogate the thrombin feed-forward loop may reduce incidence of treatment failure.

In conclusion, the inventors show plasma from HIT patients, in presence of platelet agonists together with therapeutic-concentration heparin, markedly increased the PP surface mediated through FcγRIIa. This mechanism may contribute to the hypercoagulability that causes the high thrombotic morbidity and mortality associated with HIT. Moreover, the inventors show that the flow cytometry-based PP assay of the invention can accurately differentiate HIT from thrombocytopenia due to other causes, laying the foundations for a novel, rapid yet accurate HIT diagnostic assay that can be adapted for diagnosis of other immune-thrombotic conditions.

### Vaccine-Induced Immune Thrombotic Thrombocytopenia

### Materials and Methods

### Study Approval

Human studies were approved by Concord Repatriation General Hospital (CRGH) Human Research Ethics Committee (HREC/18/CRGH/294) and (X21-0160, 2021/ETH00945). Healthy donors gave written informed consent.

### Study Cohort

Citrated plasma from 47 Australian patients referred for confirmatory VITT testing for suspected VITT after ChAdOx1 nCov-19 vaccination formed the development cohort. Data collected included time to onset of symptoms, platelet count, D-dimer, site of thrombosis and treatment initiation. All patients underwent anti-PF4 testing with PF4/heparin ELISA (Asserachrom HPIA IgG Assay, Stago Diagnostics) and VITT functional testing via standard (no exogenous PF4) serotonin-release assay (SRA). Patients were grouped as (1) confirmed VITT if fulfilling all 5 criteria of (i) vaccine within 4-42 days of symptom onset, (ii) thrombocytopenia, (iii) D-dimer >5×upper limit normal (ULN), (iv) anti-PF4/polyanion ELISA+ve and (v) SRA+ve; (2) VITT negative if (i) ELISA-ve, (ii) SRA-ve and (iii) clinically adjudicated to not be VITT; (3) “ELISA false positive” if (i) ELISA+ve, (ii) SRA or Multiplate multiple electrode aggregometry negative and (iii) normal platelet count. Comparison was made with plasma from 32 healthy individuals. Performance of the flow cytometry assay was assessed in a validation cohort of 99 VITT cases clinically adjudicated by members of the Thrombosis and Haemostasis Society of Australia and New Zealand (THANZ) advisory group based on clinicopathological criteria.

### Healthy Volunteers

Healthy donors recruited at CRGH and ANZAC Research Institute (Sydney, Australia) were screened to identify FcγRIIa responders using light transmission aggregometry as described.⁴⁷ Blood was collected from the antecubital fossa into 3.2% citrate tubes using a 21 G butterfly needle. The initial 3 mL of blood was discarded.

### Procoagulant Platelet Assay

Citrated whole blood (13 μL) was incubated with and without 5 μM SFLLRN (Auspep), unfractionated heparin (0.5-100 U/mL, Pfizer), ChAdOx1 nCoV-19 (1:2000 (v/v), AstraZeneca). In some experiments, blood was pre-treated with 10 μg/mL monoclonal antibody against FcγRIIa IV.3 (StemCell Technologies) or 10 mg/mL IVIg (Privigen) for 15 minutes prior to the reaction. In some experiments, fondaparinux (1.2-100 μg/mL, Arixtra), SARS-CoV-2 spike protein (20 μg/mL HexaPro), or 25 μg/mL native human PF4 purified from expired platelet concentrate⁴⁸ was added. 5 μL citrated plasma was added to reaction mix for 10 minutes, with 2.5 mM Gly-Pro-Arg-Pro peptide (Sigma Aldrich) and 2.5 mM calcium chloride in Hanks' balanced salt solution (HBSS, pH 7.35) in a 50 μL reaction. The reaction was stopped by further dilution with HBSS, followed by staining with antibodies to CD45 (HI30) (StemCell Technologies), CD41a (HIP8) (BD Biosciences), CD62P (Psel.KO2.3) (eBioscience)—or isotype control (eBioscience), and GSAO or control compound GSCA (4-(N—(S-glutathionylacetyl)amino)benzoic acid). Then, samples were fixed with PAMFix (Platelet Solutions Ltd), centrifuged and resuspended, prior to analysis on a BD LSRFortessa X-20 or BD FACSCanto II cytometer with acquisition of 3000-7000 platelet events. Plasma from HIT patients and healthy individuals were tested in parallel as positive and negative controls, respectively.

### Serotonin Release Assay

Serotonin release assay (SRA) was performed at NSW Health Pathology Haematology Randwick, Sydney according to the original method with minor modifications⁴⁹ including preincubation with IV.3 (10 μg/mL), and in some cases, addition of PF4 (18 μg/mL) purified from expired human platelets.⁴⁸

### Statistical Analyses

Statistical analyses were performed using GraphPad Prism 9.2 (La Jolla, USA), with statistical significance set at p<0.05. Experimental data are presented as mean±standard deviation. Details of analyses are included in figure legends.

### Results

Plasma from VITT Patients Sensitises Healthy Donor Platelets to Become Procoagulant in the Presence of Platelet Agonists

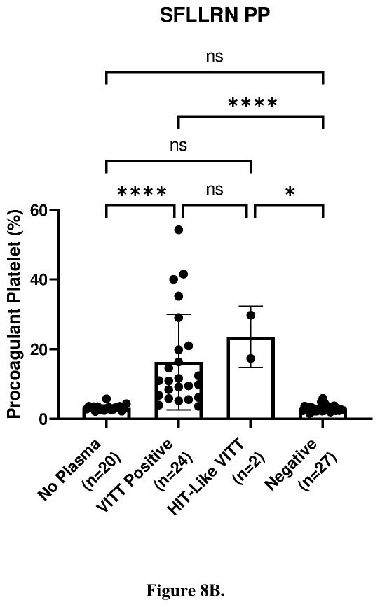

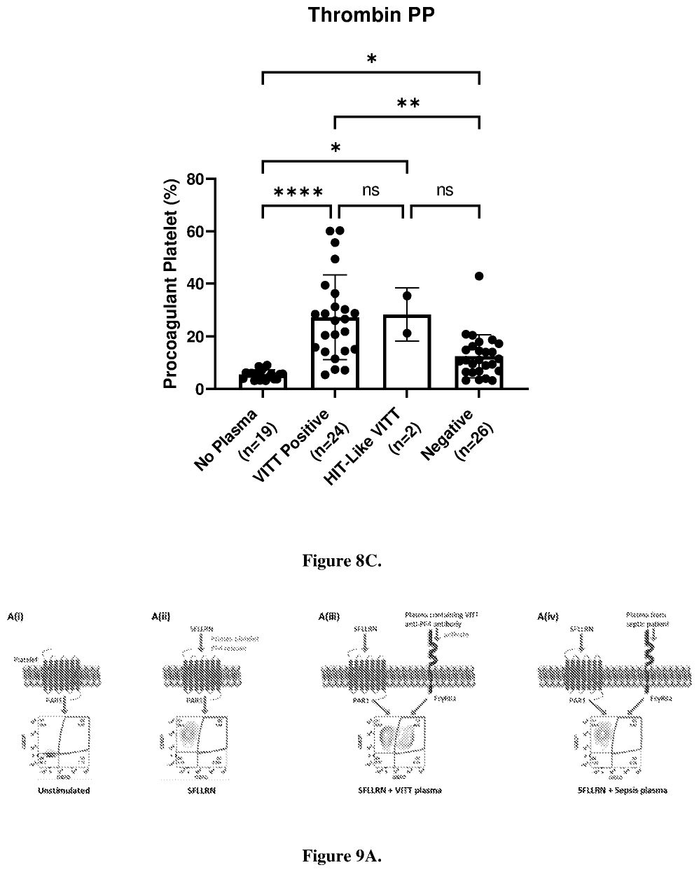

Procoagulant platelets were identified using a whole blood flow cytometry-based assay as CD41a+/CD45− events that were marked with both the cell necrosis marker GSAO-AF647, and the platelet activation marker P-selectin (GSAO+/CD62P+) as previously described. Pre-incubation with plasma from patients with VITT markedly increased the proportion of procoagulant platelets in the whole blood of healthy donors following stimulation with a weak platelet agonist SFLLRN (5 μM). Representative flow cytometry plots from a healthy donor comparing no plasma with VITT positive, HIT-like VITT (heparin-enhancing VITT) or negative plasma indicate that addition of VITT plasma altered the sub-population profile of the platelets with a marked increase in procoagulant (GSAO+/CD62P+) platelets (FIG. 8A). There was a significantly higher proportion of procoagulant platelets in the presence of VITT plasma compared with negative plasma (16.34±13.74 vs 2.988±0.9564, P<0.0001) and no plasma (16.34±13.74 vs 3.194±0.8185, P<0.0001) (FIG. 8B). Plasma from patients with HIT-like VITT (heparin-enhancing VITT) induced a significantly higher procoagulant platelet response than negative plasma (23.60±8.768 vs 2.988±0.9564, P=0.0244) although it did not reach statistical significance when compared with no plasma (23.60±8.768 vs 3.194±0.8185, P=0.0679) (FIG. 8B). Importantly, plasma from VITT-negative patients did not increase the baseline procoagulant platelet response on donor platelets in the absence of exogenous plasma (2.988±0.9564 vs 3.194±0.8185, P>0.9999) (FIG. 8B).

### Characteristics of Patients Suspected of VITT: Development Cohort

Forty-seven patients (19 female, 28 male) with a median age of 70 years (range 34 to 95) referred for confirmatory VITT testing with thrombosis after ChAdOx1 nCoV-19 vaccination were analyzed for the development cohort. Demographic data are summarized in Table 4. Patients with confirmed VITT were significantly younger (65±12.6 vs 74±14.0 years, p=0.017) with lower platelet count (45±32.0 vs 130.5±72.3×10⁹/L, p<0.0001) and higher D-dimer levels (40±50.5 vs 14.4±21.6 fold-increase above ULN, p=0.006) compared to VITT negative patients. No significant differences in gender distribution, timing of presentation relative to vaccination or incidence of thrombosis was observed between confirmed VITT and VITT negative patients. All 23 confirmed VITT patients had thrombosis including cerebral venous sinus thrombosis (4 patients), splanchnic vein thrombosis (4 patients), pulmonary embolism (12 patients) and deep vein thrombosis (10 patients). Twenty-one of 23 VITT patients received IVIg. Forty-six of 47 samples were collected for VITT testing prior to treatment initiation.

TABLE 4
Laboratory and clinical characteristics of patients
suspected of VITT in the development cohort.

| | | VITT | VITT | ELISA | |
|---|---|---|---|---|---|
| Variables | All | Positive | Negative | False Positive | p value* |
| n | 47 | 23 | 20 | 4 | |
| Gender | | | | | 0.350 |
| Female | 19 | 11 | 6 | 2 | |
| Male | 28 | 12 | 14 | 2 | |
| Age (years) | 70 ± 14.04 | 65 ± 12.63 | 74 ± 14.03 | 69 ± 17.91 | 0.017 |
| | [34-95] | [44-87] | [34-95] | [45-88] | |
| Days post-vaccine | 10 ± 8.94 | 9 ± 7.49 | 12 ± 10.96 | 8.5 ± 4.72 | 0.912 |
| | [1-39] | [4-31] | [1-39] | [7-17] | |
| Platelet count | 85 ± 76.13 | 45 ± 31.98 | 130.5 ± 72.30 | 232 ± 32.53 | <0.0001 |
| (10⁹/L) | [7-337] | [7-140] | [34-337] | [209-255] | |
| D-dimer (fold- | 33.76 ± 43.76 | 40 ± 50.53 | 14.4 ± 21.62 | <5x ULN in 3 | 0.006 |
| change over ULN) | [2-228] | [3.7-228] | [2.0-75.0] | patients | |
| Thrombosis | 45 | 23 | 19 | 3 | 0.465 |

*Fisher's exact test (categorical variables) or Mann-Whitney U test (continuous variables) between VITT positive and VITT negative groups.
Continuous variables are expressed as median ± SD [range];
ULN, upper limit of normal

Plasma from VITT Patients Sensitizes Healthy Donor Platelets to Become Procoagulant

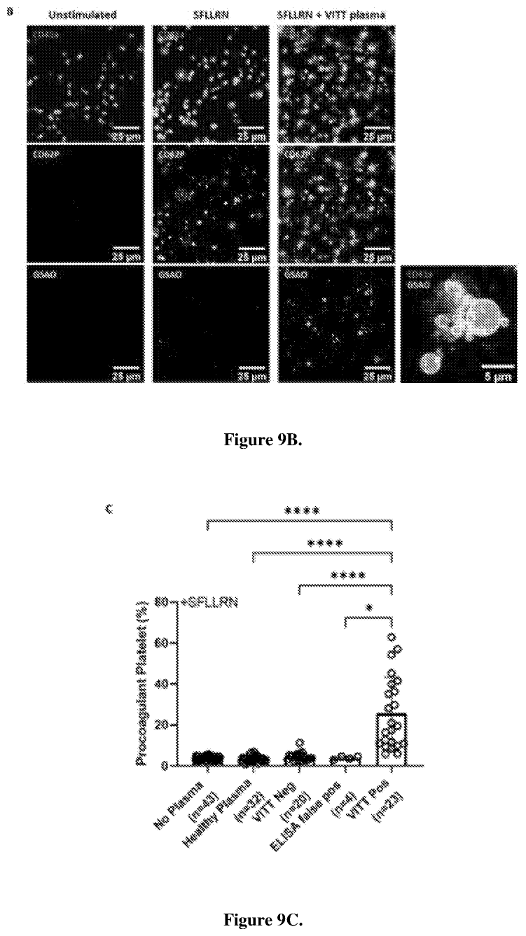

PP in whole blood reactions were detected by flow cytometry and defined as CD41a-positive (platelet-specific marker) events expressing both GSAO and P-selectin. In proof of principle experiments, donor blood treated with PAR1 agonist SFLLRN causes release of platelet alpha granules represented by P-selectin expression, with minimal PP formed (FIGS. 9A and B). Exposing healthy blood to both SFLLRN and plasma containing VITT anti-PF4 antibodies, but not plasma from a septic patient, results in a synergistic effect whereby a dramatic increase in the PP proportion is observed by flow cytometry (FIG. 9A) and evidence of ballooning with GSAO uptake by microscopy (FIG. 9B). In the development cohort of 47 patients, minimal PP (3.86±0.97%) were generated by healthy donor blood treated with SFLLRN (FIG. 9C). Addition of plasma from healthy individuals (3.57±1.36%), or ChAdOx-1 nCoV-19 vaccinated patients with thrombocytopenia and thrombosis but without detectable anti-PF4 antibodies (VITT-negative, 4.50±2.09%), or ELISA false positive, (3.93±0.78%), did not increase the procoagulant response in donor platelets. However, the addition of plasma from confirmed VITT patients significantly increase the PP response (25.67±17.68%) when compared to no plasma (p<0.0001), healthy (p<0.0001), VITT-negative (p<0.0001) or ELISA false positive (p=0.0381) samples (FIG. 9C). The PP proportions induced by VITT plasma were variable between patients (range: 5.74% to 62.9%).

### PP Response Induced by VITT Plasma is Suppressed by Heparin

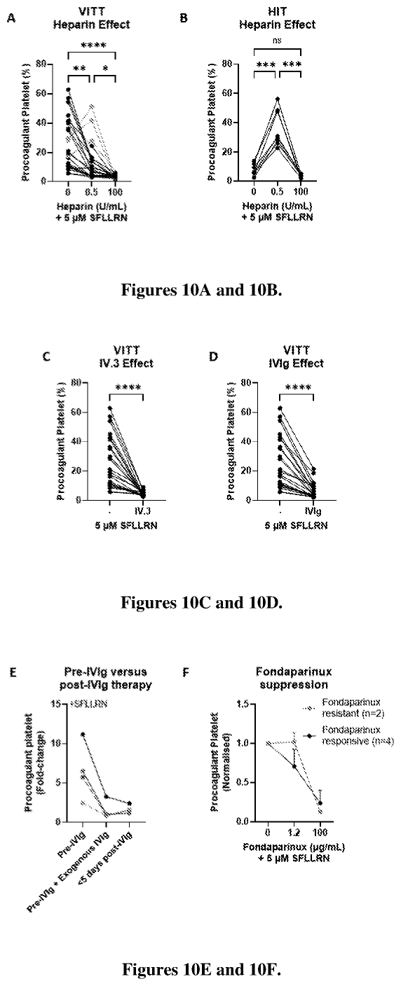

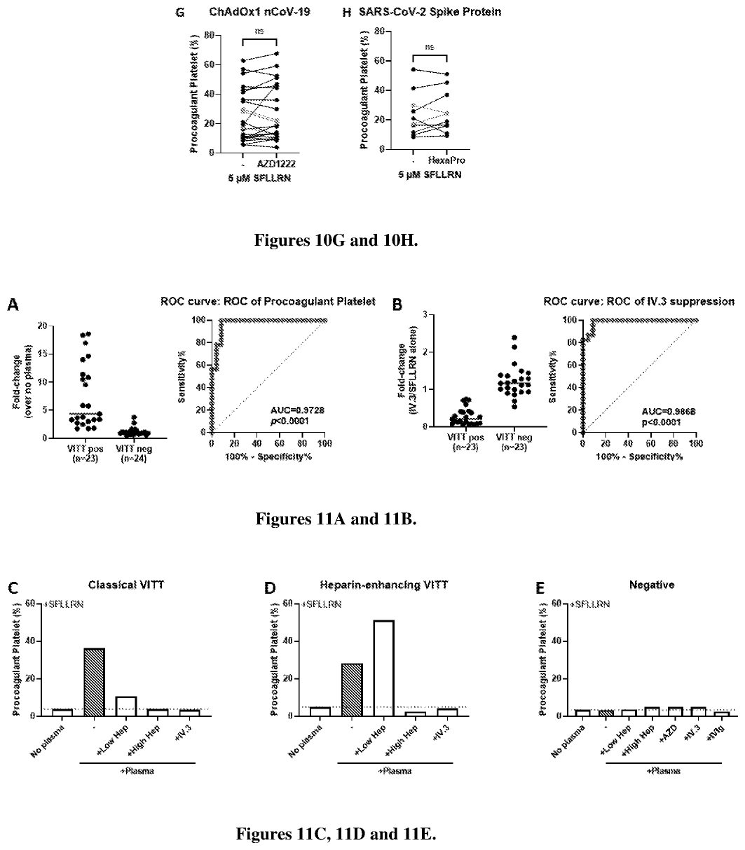

To explore the effect of charged molecule on VITT-induced PP response, low- (0.5 U/mL) and high-dose (100 U/mL) heparin was added to the reaction. Low-dose heparin significantly reduced the PP response induced by the majority of VITT plasma (12.48±12.84% vs 25.67±17.68%, p=0.0096) (FIG. 10A). The inhibitory effect of low-dose heparin on VITT plasma-induced PP response was variable, 0.14-1.02-fold (0.39±0.21-fold) change from plasma without heparin, in presence of low-dose heparin. Interestingly, three of 23 patients showed enhanced PP response with low-dose heparin (FIG. 10A) which was reminiscent of the procoagulant response induced by HIT plasma (FIG. 10B). In all cases, the PP response was completely abolished in presence of high-dose heparin (VITT: 3.54±1.41% vs 25.67±17.68%, p<0.0001, HIT: 3.48±1.02% vs 42.80±12.10%, p<0.0001).

### VITT-Induced PP Response Requires FcγRIIa and Inhibitable by IVIg

Donor platelets were pre-treated with FcγRIIa function-blocking monoclonal antibody IV.3 or IVIg before exposure to patient plasma to demonstrate that the PP response is mediated through FcγRIIa and induced by antibodies. Regardless of the extent of PP response, the PP proportion was significantly reduced to baseline levels in presence of IV.3 (4.49±1.74 vs 25.79±17.79, p<0.0001, FIG. 10C). Although IVIg significantly decreased the PP proportions induced by VITT plasma (6.43±5.23 vs 25.79±17.79, p<0.0001, FIG. 10D), the extent of inhibition varies between patients whereby partial inhibition was observed in some patients.

### In Vitro PP Response to Clinical Therapies

Addition of exogenous IVIg variably reduced the PP response induced by VITT plasma. As IVIg is recommended for treating VITT^{33,34} the inventors evaluated whether the in vitro response to IVIg could inform on the in vivo clinical response to IVIg therapy in four confirmed cases collected prior to and within five days of IVIg infusion (FIG. 10E). The post-IVIg samples showed a procoagulant response similar to the pre-IVIg sample with addition of exogenous IVIg. Due to the similarities between VITT and HIT, non-heparin anticoagulants such as synthetic factor Xa inhibitor (modeled after the heparin pentasaccharide sequence), fondaparinux are used in VITT.^{33,34} Treatment of SRA-confirmed HIT with fondaparinux resulted in mixed outcomes⁵⁰ and similar “resistance” may also occur in VITT. The inventors compared the effect of exogenous fondaparinux on the PP response induced by pre-treatment VITT plasma from six patients treated with fondaparinux. At therapeutic concentration of fondaparinux (1.2 μg/mL), the inventors observed a decreasing trend in the PP response induced by plasma from four patients who clinically responded to fondaparinux therapy (FIG. 10F). In contrast, no change in PP response was seen with fondaparinux when tested with plasma samples from patients who did not respond clinically. Maximum inhibition of the PP response was achieved in all patients at 100 μg/mL fondaparinux.

Addition of ChAdOx1 nCoV-19 and SARS-CoV-2 Spike Protein Resulted in Inconsistent Effect on Procoagulant Response

Exogenous ChAdOx1 nCoV-19 did not induce a procoagulant response in absence of VITT plasma. Addition of ChAdOx1 nCoV-19 enhanced the PP response in some VITT patients but had no effect in others (FIG. 10G). The procoagulant response generated by three patients with heparin-enhancing VITT was reduced in presence of the vaccine. Overall, there was no significant change in the PP response induced by 23 VITT plasma in presence of vaccine (27.12±19.05 vs 25.80±17.81, p=0.6273, FIG. 10G).

HexaPro is a recombinant form of the SARS-CoV-2 spike protein containing six amino acid substitutions for stable expression.⁵¹ Inclusion of the spike protein did not significantly change the PP response generated by 10 VITT patient plasma (25.48±14.41 vs 23.64±14.77, p=0.3223, FIG. 10H) although some increase in the procoagulant response was observed in certain patients. A positive but non-significant trend (Pearson r=0.538, p=0.109) was observed between the effect of ChAdOx1 nCoV-19 and HexaPro on VITT plasma-induced PP response.

### Diagnostic Utility of PP Assay

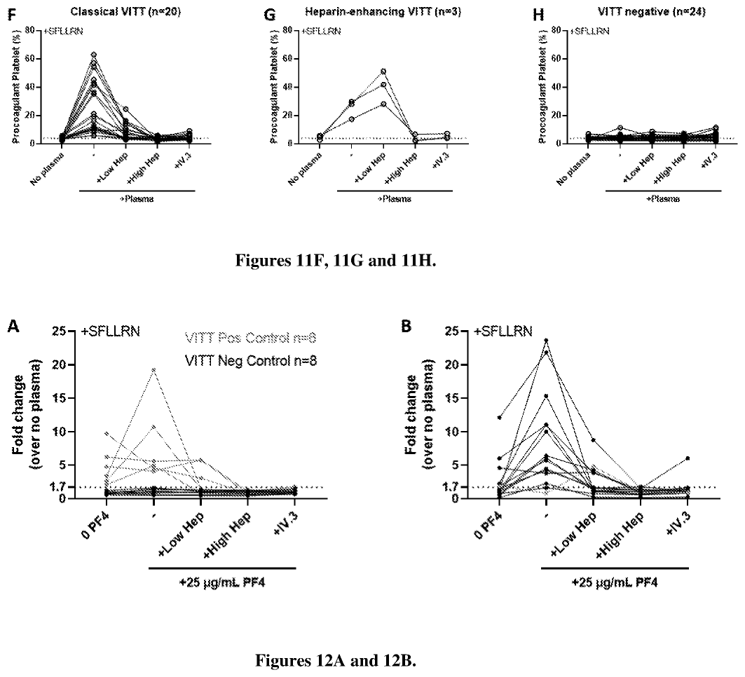

To evaluate the diagnostic potential, a receiver operating characteristics curve (ROC) was generated using plasma from confirmed VITT patients (n=23) who tested positive on both ELISA and SRA, and VITT-negative patients (n=24) comprising of ELISA-negative and SRA-negative patients and ELISA false positive patients who are non-thrombocytopenic and SRA-negative. The area under the curve (AUC) was 0.97±0.02, p<0.0001 with a cut-off of 1.7-fold increase in plasma-induced PP formation demonstrating 100% sensitivity and 91.7% specificity (FIG. 11A). Suppression of the PP response by IV.3 mediated blockade of FcγRIIa may add additional discrimination between antibody (VITT) and non-antibody (non-VITT) mediated procoagulant response. A<0.72-fold change achieved an adequate sensitivity and specificity of 91.3% with an AUC of 0.99±0.01, p<0.0001 (FIG. 11B) and identified individuals with non-antibody mediated procoagulant platelet enhancement.

Pattern of PP Response Inpatients with VITT or Controls

FIGS. 11C-H demonstrates the patterns of PP response used in reporting. A pattern that the inventors termed “classical VITT” demonstrates 1.7-fold increase in PP compared with baseline, with abrogation in presence of 100 U/mL heparin, at least a 0.72-fold decrease in presence of monoclonal antibody IV.3 and reduction in the presence of 0.5 U/mL heparin. The pattern associated with a sub-type the inventors termed “heparin-enhancing VITT” shows the same as classical VITT but with an increased PP response in presence of 0.5 U/mL heparin. An equivocal response demonstrates >1.7-fold increase induced by plasma but >0.72-fold reduction after preincubation with IV.3 regardless of other reactivity.

### Addition of PF4 to Assay in High Probability Cases Negative in Standard Flow Cytometry

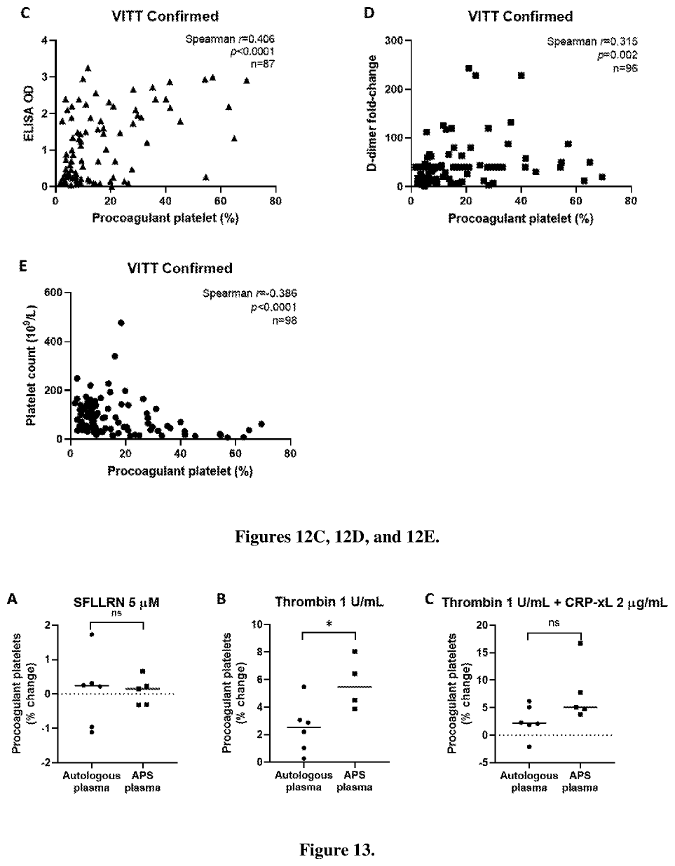

The addition of exogenous PF4 has been demonstrated to improve the sensitivity of functional platforms to detect platelet-activating antibodies in VITT.^{30,41,52,53} The inventors investigated the effect of purified human PF4 on the performance of the flow cytometry assay of the invention using plasma from confirmed VITT positive and negative cases. In positive cases, inclusion of exogenous PF4 resulted in persistent plasma-induced procoagulant response inhibited by high-dose heparin and in presence of IV.3. Exogenous PF4 did not alter the results of VITT positive (n=6) and negative (n=8) cases reported by the standard flow cytometry assay without PF4 (FIG. 12A). Plasma samples that were inconclusive or negative by the standard assay, but positive via SRA or PF4-SRA were tested with exogenous PF4 addition to flow cytometry. Two of three patients who were equivocal or inconclusive on the standard flow cytometry assay became positive while one remained equivocal with the addition of PF4 (Table 5). Three of five patients who were thought to be false negative on standard flow cytometry-(SRA-positive), became positive with PF4-enhanced flow cytometry.

TABLE 5
Laboratory and clinical data on serologically confirmed VITT patients with discrepant results on VITT testing platforms.

| | | D- | | | | | | | | | |
|---|---|---|---|---|---|---|---|---|---|---|---|
| | Platelet | dimer | | | | | | | | | Serologically |
| | count | (fold- | | | ELISA | | | | | | confirmed |
| Code | (×10⁹/L) | change) | Thrombosis | ELISA | OD | Flow Cytometry | | SRA | | Multiplate | VITT |
| P1 | 41 | 25 | Renal artery | pos | 2.4 | Negative | Classical | HIT | NT | Positive | Yes |
| | | | occlusion | | | | VITT | | | | |
| P2 | 249 | 7.2 | PE | pos | 1.8 | Negative | Heparin- | Positive | NT | Negative | Yes |
| | | | | | | | enhancing | | | | |
| | | | | | | | VITT | | | | |
| P3 | 53 | 40 | CVST, bilat | neg | 0.11 | Negative | Negative | HIT | NT | Negative | Yes |
| | | | DVT | | | | | | | | |
| P4 | 124 | 14.5 | PE, DVT | neg | 0.064 | Negative | Negative | Weak | Weak | NT | Yes |
| | | | | | | | | positive | positive | | |
| P5 | 45 | 17 | Carotid artery, | pos | 0.36 | Negative | Classical | Positive | NT | Negative | Yes |
| | | | CVST, PE, LL | | | | VITT | | | | |
| | | | arterial | | | | | | | | |
| P6 | 153 | 5 | CVST | pos | 1.34 | Equivocal | Equivocal | Weak | Equivocal | Inconclusive | Yes |
| | | | | | | | | positive | | | |
| P7 | 108 | 5.8 | MCA stroke | pos | 0.88 | Equivocal | Classical | Negative | NT | Negative | Yes |
| | | | | | | | VITT | | | | |
| P8 | 110 | 26.6 | PE | pos | 0.87 | Inconclusive | Classical | Negative | Equivocal | NT | Yes |
| | | | | | | | VITT | | (neg) | | |
| P9 | 100 | 22 | DVT | pos | 0.74 | Negative | Classical | NT | Inconclusive | NT | Yes |
| | | | | | | | VITT | | | | |

| | Platelet | D-dimer | | | | Flow Cytometry | | SRA | | | Serologically |
|---|---|---|---|---|---|---|---|---|---|---|---|
| | count | (fold- | | | ELISA | | PF4 | | PF4 | | confirmed |
| Code | (×10⁹/L) | change) | Thrombosis | ELISA | OD | Standard | Enhanced | Standard | Enhanced | Multiplate | VITT |
| P10 | 198 | NT | PE | neg | 0.14 | Classical | NT | Positive | NT | Positive | Yes |
| | | | | | | VITT | | | | | |
| P11 | 89 | 28.75 | PE, DVT | neg | 0.16 | Classical | Classical | Positive | NT | Negative | Yes |
| | | | | | | VITT | VITT | | | | |
| P12 | 16 | 44 | ICA | neg | 0.15 | Classical | NT | Positive | NT | NT | Yes |
| | | | | | | VITT | | | | | |
| P13 | 46 | 31 | Popliteal | neg | 0.18 | Classical | NT | Positive | NT | NT | Yes |
| | | | | | | VITT | | | | | |
| P14 | 125 | 16 | DVT | pos | 0.6 | Classical | NT | Negative | Positive | Positive | Yes |
| | | | | | | VITT | | | | | |
| P15 | 220 | 32.5 | PE, DVT | weak | 0.38 | Classical | NT | Negative | Negative | Positive | Yes |
| | | | | pos | | VITT | | | | | |
| P16 | 138 | 16 | MCA stroke | pos | 0.215 | Classical | NT | Negative | Negative | Negative | Yes |
| | | | | | | VITT | | | | | |
| P17 | 42 | 119.8 | PE | pos | 2.56 | Classical | NT | Negative | Negative | NT | Yes |
| | | | | | | VITT | | | | | |
| P18 | 38 | 40 | Portal vein | pos | 2.1 | Classical | NT | Negative | Positive | NT | Yes |
| | | | thrombosis | | | VITT | | | | | |
| P19 | 14 | 40 | CVST, bilat | pos | 1.21 | Classical | NT | Negative | Positive | NT | Yes |
| | | | ICA | | | VITT | | | | | |
| P20 | 161 | 62 | Proximal DVT | weak | 0.29 | Classical | NT | Negative | Negative | Negative | Yes |
| | | | | pos | | VITT | | | | | |
| P21 | 25 | 40 | CVST, bilat | pos | 1.46 | Classical | NT | Negative | Negative | Positive | Yes |
| | | | ICA, PE, portal | | | VITT | | | | | |
| | | | vein thrombosis | | | | | | | | |
| P22 | 86 | 120 | Splanchnic vein | pos | 1.73 | Classical | NT | Negative | NT | NT | Yes |
| | | | thrombosis | | | VITT | | | | | |
| P23 | 95 | 40 | Soleal vein | pos | 1.24 | Classical | NT | Negative | Positive | Positive | Yes |
| | | | thrombosis | | | VITT | | | | | |
| P24 | 128 | 15.2 | Proximal DVT | neg | NA | Classical | NT | Negative | Negative | Negative | Yes |
| | | | | | | VITT | | | | | |
| P25 | 62 | 19.8 | PE | pos | 2.9 | Classical | NT | Negative | Positive | Negative | Yes |
| | | | | | | VITT | | | | | |
| P26 | 77 | 14.8 | Bilat PE | pos | 2.39 | Classical | NT | Negative | NT | NT | Yes |
| | | | | | | VITT | | | | | |
| P27 | 125 | 66 | PE | neg | 0.07 | Classical | NT | Negative | Positive | NT | Yes |
| | | | | | | VITT | | | | | |

### Evaluation of Utility in Case Ascertainment

Analysis of 108 cases referred for functional testing within the first 2.5 months of VITT in Australia (Table 5), 41 cases were concordant between ELISA, SRA and the flow cytometry assay of the invention. Four cases who tested positive on both standard SRA and flow cytometry platforms were ELISA-negative and were classified as VITT by Australian VITT advisory that would have been missed using ELISA criteria alone. 14 patients were positive by the flow cytometry assay of the invention that were missed by initial standard SRA testing. 13 of the 14 were VITT serologically supported by either ELISA, PF4-enhanced SRA or multiplate as functional assays. One of 14 was clinically adjudicated to be VITT without additional serological support indicating they were likely to be true VITT cases. The flow cytometry assay of the invention enabled 7.4% cases in this cohort that would be missed by ELISA and 14.8% cases missed by standard SRA, to be classified as VITT.

### Performance of Test in Cases Referred for VITT Testing

Of 99 cases adjudicated as VITT by THANZ VITT advisory group members (Table 6), 98 underwent flow cytometry testing according to the invention. 4 patients had no serological support by either ELISA or any functional test platform. 76 of the remaining 94 were positive on flow cytometry without PF4 enhancement. 15 of the 18 individuals classified as negative, inconclusive or equivocal returned a positive result with PF4 enhancement (FIG. 12B). Three remained equivocal (increased procoagulant response to plasma without IV.3 suppression). Overall, 93% of patients with clinic-pathological adjudication of VITT were detectable by performance of SFLLRN flow cytometry assay of the invention with sequential testing of negative and equivocal samples with addition of exogenous PF4. PP formation demonstrated a positive correlation with anti-PF4 antibody (Spearman r=0.406, p<0.0001, FIG. 12C) and D-dimer (Spearman r=0.315, p=0.002, FIG. 12D) levels but a negative correlation against platelet count (Spearman r=−0.386, p<0.0001, FIG. 12E).

TABLE 6
Laboratory and clinical characteristics of adjudication
confirmed VITT patients referred for VITT testing.

| Variables | Adjudication confirmed VITT |
|---|---|
| n | 99 |
| Female | 55 |
| Male | 44 |
| Age (years) | 63 ± 14.89 [19-91] |
| Days post-vaccine | 10 ± 8.60 [2-43] |
| Platelet count (10⁹/L) | 70 ± 73.12 [7-477] |
| D-dimer (fold-change over ULN) | 40 ± 44.69 [1-243] |
| Thrombosis | 99 |
| Cerebral venous sinus thrombosis | 28 |
| Splanchnic thrombosis | 19 |
| Pulmonary embolism | 40 |
| Deep vein thrombosis | 26 |
| Other | 21 |
| Mortality | 7 |

Continuous variables are expressed as median ± SD [range]; ULN, upper limit of normal.

### Discussion

Considering the pathophysiological similarities between HIT and VITT, the inventors adapted the method for diagnosing HITT discussed above to ascertain its diagnostic utility in a development cohort of 47 patients suspected of VITT and confirmed this within sequential patients referred for VITT functional testing within Australia.

As hypothesized, the level of PP formation with low-dose SFLLRN stimulation was minimal in healthy donor whole blood, despite near complete alpha granule release. There was no increase in procoagulant response with addition of negative control plasmas that lack ITAM signaling capacity, while plasma with HIT or VITT antibodies capable of activating ITAM signaling demonstrated a procoagulant response consistent with the known synergistic effect of combined GPCR and ITAM signaling on formation of this platelet sub-population.

Evaluation of this assay using plasma samples from ELISA-positive and SRA-positive confirmed VITT patients confirmed that the assay of the invention detected a plasma-induced FcγRIIa dependent PP response in donor platelets that is suppressible by high-dose heparin and intravenous immunoglobulin. The procoagulant response was not observed in vaccinated patients who did not have the anti-PF4 antibodies and are SRA-negative, or those with detectable non-pathological anti-PF4 antibodies (ELISA-positive, SRA-negative, non-thrombocytopenic patients), signifying specificity. Inclusion of a criteria for suppression of plasma induced procoagulant activity by monoclonal antibody IV.3 increased the specificity. Two other flow cytometry assays have recently been described that detect VITT antibodies—PIFPA by Handtke and colleagues⁵⁴ performed on hirudinated donor whole blood with addition of exogenous PF4 with P-selectin MFI as an output; and a washed platelet PP assay, by Althaus and colleagues,⁴¹ again supplemented with exogenous PF4 with combination annexin V and P-selectin as the readout. The assay of the invention, of PP response induced by patient plasma in citrated whole blood from high responsive donors represents an additional functional platform to HIPA, SRA, PIFPA and multiplate for detection of platelet-activating VITT antibodies.

Increasingly, data suggests that VITT is a spectrum disorder. There is variation in anti-PF4 antibody levels, and the first NEQAS QAP exercise demonstrated significant variability in functional assay detection of VITT antibodies demonstrating need for ongoing refinement of VITT diagnostic assays.⁵⁵ Application functional assays to a large cohort of patients referred for VITT functional testing in Australia demonstrates heterogeneity. Some patients that had platelet-activating antibodies that were detectable by flow cytometry were not detected by SRA and vice versa suggesting heterogeneity in the pathophysiology of VITT antibodies.

Unlike HIT, inclusion of low-dose heparin diminished the PP response induced by VITT plasma except for three patients with a heparin-dependent exacerbation of procoagulant response. Huynh and colleagues demonstrated VITT antibodies share similar binding sites as heparin on PF4.³⁵ Addition of heparin presumably competes with VITT antibodies for PF4 binding, resulting in an abrogated platelet-activating effect with complete abrogation at high-dose heparin. The variability in the effect of low-dose heparin whereby partial suppression was observed in some patients while full suppression was achieved in others is consistent with published reports. For instance, 5 of 241 and 3 of 812 VITT sera remained positive in platelet-activation assays in presence of 0.2 U/mL heparin.

Through molecular simulations, Baker and colleagues revealed that the negatively charged surface of the ChAdOx1 viral capsid interacts with the positively charged PF4, presumably leading to exposure of neo-epitopes on PF4, thereby triggering an immune response against PF4.⁵⁶ Consistent with this observation, Greinacher and colleagues demonstrated the interactions between PF4 and ChAdOx1 nCoV-19 via super-resolution microscopy.⁵⁷ Taken together, the vaccine components may be a co-factor that mimics the action of heparin in altering the conformation of PF4 causing exposure of neo-epitopes on PF4 which then triggers an anti-PF4 immune response. However, at the concentration tested here, the inventors observed an inconsistent effect of ChAdOx1 nCoV-19 on the PP response induced by VITT plasma.

The inventors' data suggests that measurement of PP response induced by VITT plasma may offer personalized predictors for patients. The heightened PP response generated by VITT plasma was positively associated with anti-PF4 antibody levels and trended with decreasing platelet count at time of diagnosis. Extent of thrombocytopenia has been associated with significant mortality risk whereby for every 50% reduction in baseline platelet count there is a 1.7-fold increased risk of death related to VITT.⁵⁸ The inventors speculate that the individuals with a “heparin enhancing” response would be at risk of thrombus progression in presence of heparin, similar to HIT patients and should avoid future heparin exposure. The concordance observed between the in vitro effect of IVIg and fondaparinux on VITT-induced PP response, and the clinical response of these patients to IVIg and fondaparinux therapy, may help identify individuals who would benefit from change in anticoagulant or additional immune-suppression therapy. Additionally, it can be used to monitor persistence or resolution of the platelet-activating properties of VITT antibodies which may be discordant to the persistence in the ELISA, to guide duration of anticoagulation.⁵⁹

The assay of the invention offers advantages over other platelet-activating assays. The use of standard citrated whole blood provides a more physiological environment for the reaction to take place and requires minimal manipulation of donor platelets. A small volume of plasma (5 μL per test condition) and the use of standard flow cytometers add to its practicality. Notably, the assay of the invention was able to detect the majority of VITT cases without the requirement for exogenous PF4. This is an important consideration due to potential variability introduced by addition of exogenous PF4 as its oligomerization state varies with preparation methods,^{61,62} in addition to the cost associated with purchase or extraction of PF4. The nature of the PP being a subset of the whole platelet population rather than an “all-or-nothing” response characteristic of alpha and dense granule release detection methods enables detection of heparin enhancement in the plasma with strongly activating heparin-independent antibodies. In assays with 100% dense granule release with serum alone, detection of further increase in presence of low-dose heparin is not possible.

In conclusion, induction of FcγRIIa dependent PP response by patient plasma that is suppressible by heparin and IVIg is highly indicative of VITT in the correct clinical circumstance. The assay of the invention involves priming platelets from known FcγRIIa responsive donors with a GPCR agonist to potentiate the ITAM signaling from platelet-activating immune complexes, which results in a sensitive and specific assay. This represents a functional platform with proven translational capacity into diagnostic laboratories to identify patients with platelet-activating antibodies and potentially predict treatment responses.

### Antiphospholipid Syndrome

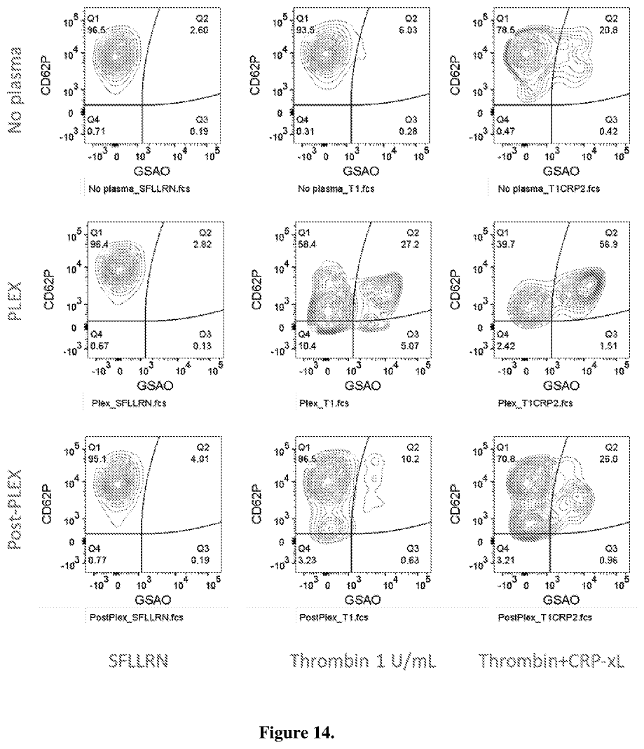

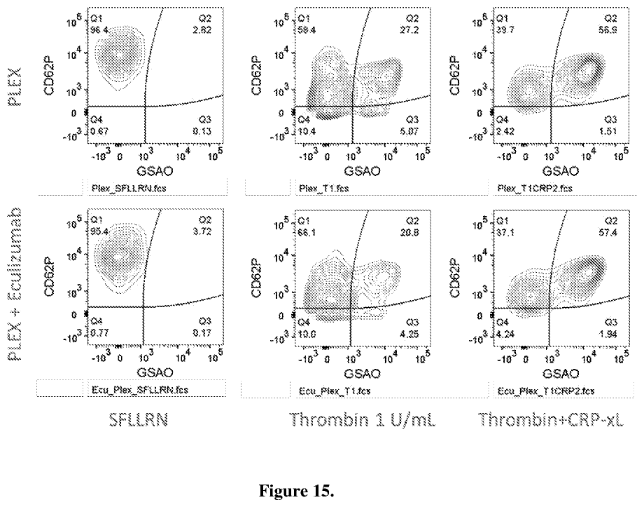

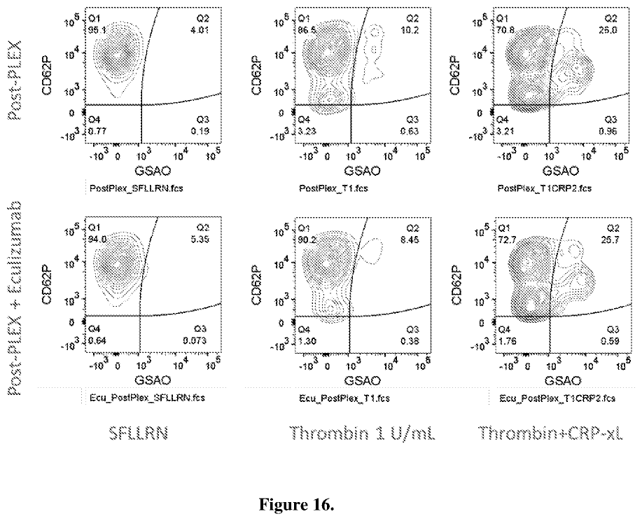

Citrated whole blood (13 μL) was incubated with platelet agonists and plasma (5 μL) from healthy donors or patients, for 10 minutes with final concentrations of 2.5 mM Gly-Pro-Arg-Pro peptide (Sigma-Aldrich) and 2.5 mM calcium chloride in a 50 μL reaction. Agonists included CRP-xL, ADP (Helena Laboratories), thrombin receptor-activating peptide (SFLLRN, Auspep), and bovine thrombin (Sigma-Aldrich). In some experiments, blood was pre-treated with 50 μg/mL eculizumab (monoclonal antibody against complement protein C5 (Soliris)) for 15 minutes prior to the reaction. The reaction was stopped by further dilution with HBSS, followed by staining with antibodies to CD45 (HI30) (StemCell Technologies), CD41a (HIP8) (BD Biosciences), CD62P (Psel.KO2.3) (eBioscience)—or isotype control (eBioscience), and GSAO or control compound GSCA (4-(N—(S-glutathionylacetyl)amino)benzoic acid). Then, samples were fixed with PAMFix (Platelet Solutions Ltd), centrifuged and resuspended, prior to analysis on a BD LSRFortessa X-20 or BD FACSCanto II cytometer with acquisition of 10000 platelet events. Statistical analyses were performed using GraphPad Prism 9, with statistical significance set at p<0.05. See FIGS. 13 to 16.

### REFERENCES

1. Tutwiler V, Madeeva D, Ahn H S, et al. Platelet transactivation by monocytes promotes thrombosis in heparin-induced thrombocytopenia. Blood. 2016; 127(4):464-472.
2. Suvama S, Espinasse B, Qi R, et al. Determinants of PF4/heparin immunogenicity. Blood. 2007; 110(13):4253-4260.
3. Tomer A, Masalunga C, Abshire T C. Determination of heparin-induced thrombocytopenia: a rapid flow cytometric assay for direct demonstration of antibody-mediated platelet activation. Am J Hematol. 1999; 61(1):53-61.
4. Pasalic L, Wing-Lun E, Lau J K, et al. Novel assay demonstrates that coronary artery disease patients have heightened procoagulant platelet response. J Thromb Haemost. 2018; 16(6):1198-1210.
5. Lo G K, Juhl D, Warkentin T E, Sigouin C S, Eichler P, Greinacher A. Evaluation of pretest clinical score (4 T's) for the diagnosis of heparin-induced thrombocytopenia in two clinical settings. J Thromb Haemost. 2006; 4(4):759-765.
6. Favaloro E J, McCaughan G, Mohammed S, et al. HIT or miss? A comprehensive contemporary investigation of laboratory tests for heparin induced thrombocytopenia. Pathology. 2018; 50(4):426-436.
7. Favaloro E J, Mohammed S, Donikian D, et al. A multicentre assessment of contemporary laboratory assays for heparin induced thrombocytopenia. Pathology. 2021; 53(2):247-256.
8. Worthington R E, Carroll R C, Boucheix C. Platelet activation by CD9 monoclonal antibodies is mediated by the Fc gamma II receptor. Br J Haematol. 1990; 74(2):216-222.
9. Morel-Kopp M-C, Mullier F, Gkalea V, et al. Heparin-induced multi-electrode aggregometry method for heparin-induced thrombocytopenia testing: communication from the SSC of the ISTH. J Thromb Haemost. 2016; 14(12):2548-2552.
10. Rauova L, Poncz M, McKenzie S E, et al. Ultralarge complexes of PF4 and heparin are central to the pathogenesis of heparin-induced thrombocytopenia. Blood. 2005; 105(1):131-138.
11. Tardy-Poncet B, Piot M, Chapelle C, et al. Thrombin generation and heparin-induced thrombocytopenia. J Thromb Haemost. 2009; 7(9):1474-1481.
12. Chilver-Stainer L, Lämmle B, Alberio L. Titre of anti-heparin/PF4-antibodies and extent of in vivo activation of the coagulation and fibrinolytic systems. Thromb Haemost. 2004; 91(2):276-282.
13. Cines D B, Yarovoi S V, Zaitsev S V, et al. Polyphosphate/platelet factor 4 complexes can mediate heparin-independent platelet activation in heparin-induced thrombocytopenia. Blood Adv. 2016; 1(1):62-74.
14. Cai Z, Zhu Z, Greene M I, Cines D B. Atomic features of an autoantigen in heparin-induced thrombocytopenia (HIT). Autoimmun Rev. 2016; 15(7):752-755.
15. Baluwala I, Favaloro E J, Pasalic L. Therapeutic monitoring of unfractionated heparin-trials and tribulations. Expert Rev Hematol. 2017; 10(7):595-605.
16. Polgár J, Eichler P, Greinacher A, Clemetson K J. Adenosine diphosphate (ADP) and ADP receptor play a major role in platelet activation/aggregation induced by sera from heparin-induced thrombocytopenia patients. Blood. 1998; 91(2):549-554.
17. Norgard N B, Saya S, Hann C L, Hennebry T A, Schechter E, Dale G L. Clopidogrel attenuates coated-platelet production in patients undergoing elective coronary catheterization. J Cardiovasc Pharmacol. 2008; 52(6):536-539.
18. Norgard N B, Hann C L, Dale G L. Cangrelor attenuates coated-platelet formation. Clin Appl Thromb Hemost. 2009; 15(2):177-182.
19. Kirkpatrick A C, Vincent A S, Dale G L, Prodan C I. Clopidogrel use and smoking cessation result in lower coated-platelet levels after stroke. Platelets. 2020; 31(2):236-241.
20. Judge H M, Buckland R J, Sugidachi A, Jakubowski J A, Storey R F. The active metabolite of prasugrel effectively blocks the platelet P2Y12 receptor and inhibits procoagulant and pro-inflammatory platelet responses. Platelets. 2008; 19(2):125-133.
21. Kotova Y N, Ataullakhanov F I, Panteleev M A. Formation of coated platelets is regulated by the dense granule secretion of adenosine 5′diphosphate acting via the P2Y12 receptor. J Thromb Haemost. 2008; 6(9):1603-1605.
22. Tomer A. A sensitive and specific functional flow cytometric assay for the diagnosis of heparin-induced thrombocytopenia. Br J Haematol. 1997; 98(3):648-656.
23. Padmanabhan A, Jones C G, Bougie D W, et al. A modified PF4-dependent, CD62p expression assay selectively detects serotonin-releasing antibodies in patients suspected of HIT. Thromb Haemost. 2015; 114(6):1322-1323.
24. Pishko A M, Lefler D S, Gimotty P, et al. The risk of major bleeding in patients with suspected heparin-induced thrombocytopenia. J Thromb Haemost. 2019; 17(11):1956-1965.
25. Linkins L A, Warkentin T E. Heparin-induced thrombocytopenia: real-world issues. Semin Thromb Hemost. 2011; 37(6):653-663.
26. Smythe M A, Koerber J M, Mattson J C. The incidence of recognized heparin-induced thrombocytopenia in a large, tertiary care teaching hospital. Chest. 2007; 131(6):1644-1649.
27. Warkentin T E, Eikelboom J W. Who is (still) getting HIT?Chest. 2007; 131(6):1620-1622.
28. Cuker A, Arepally G M, Chong B H, et al. American Society of Hematology 2018 guidelines for management of venous thromboembolism: heparin-induced thrombocytopenia. Blood Adv. 2018; 2(22):3360-3392.
29. Chen B, Vousden K A, Naiman B, et al. Humanised effector-null FcgammaRIIA antibody inhibits immune complex-mediated proinflammatory responses. Ann Rheum Dis. 2019; 78(2):228-237.
30. Greinacher A, Farner B, Kroll H, Kohlmann T, Warkentin T E, Eichler P. Clinical features of heparin-induced thrombocytopenia including risk factors for thrombosis. A retrospective analysis of 408 patients. Thromb Haemost. 2005; 94(1):132-135.
31. Martel N, Lee J, Wells P S. Risk for heparin-induced thrombocytopenia with unfractionated and low-molecular-weight heparin thromboprophylaxis: a meta-analysis. Blood. 2005; 106(8):2710-2715.
32. Giddings J C, Brookes L R, Piovella F, Bloom A L. Immunohistological comparison of platelet factor 4 (PF4), fibronectin (Fn) and factor VIII related antigen (VIIIR:Ag) in human platelet granules. Br J Haematol. 1982; 52(1):79-88.
33. Amiral J, Bridey F, Dreyfus M, et al. Platelet factor 4 complexed to heparin is the target for antibodies generated in heparin-induced thrombocytopenia. Thromb Haemost. 1992; 68(1):95-96.
34. Kelton J G, Smith J W, Warkentin T E, Hayward C P, Denomme G A, Horsewood P.

Immunoglobulin G from patients with heparin-induced thrombocytopenia binds to a complex of heparin and platelet factor 4. Blood. 1994; 83(11):3232-3239.
35. Kelton J G, Sheridan D, Santos A, et al. Heparin-induced thrombocytopenia: laboratory studies. Blood. 1988; 72(3):925-930.
36. Warkentin T E, Hayward C P, Boshkov L K, et al. Sera from patients with heparin-induced thrombocytopenia generate platelet-derived microparticles with procoagulant activity: an explanation for the thrombotic complications of heparin-induced thrombocytopenia. Blood. 1994; 84(11):3691-3699.
37. Tutwiler V, Madeeva D, Ahn H S, et al. Platelet transactivation by monocytes promotes thrombosis in heparin-induced thrombocytopenia. Blood. 2016; 127(4):464-472.
38. Suvama S, Espinasse B, Qi R, et al. Determinants of PF4/heparin immunogenicity. Blood. 2007; 110(13):4253-4260.
39. Arepally G M. Heparin-induced thrombocytopenia. Blood. 2017; 129(21):2864-2872.
40. Marchetti M, Barelli S, Zermatten M G, et al. Rapid and accurate Bayesian diagnosis of heparin-induced thrombocytopenia. Blood. 2020; 135(14):1171-1184.
41. Nellen V, Sulzer I, Barizzi G, Lämmle B, Alberio L. Rapid exclusion or confirmation of heparin-induced thrombocytopenia: a single-center experience with 1,291 patients. Haematologica. 2012; 97(1):89-97.
42. Tomer A, Masalunga C, Abshire T C. Determination of heparin-induced thrombocytopenia: a rapid flow cytometric assay for direct demonstration of antibody-mediated platelet activation. Am J Hematol. 1999; 61(1):53-61.
43. Mordakhanova E, Nevzorova T, Synbulatova G, Rauova L, Weisel J, Litvinov R. Platelet Activation in Heparin-Induced Thrombocytopenia is Followed by Platelet Death via Complex Apoptotic and Non-Apoptotic Pathways. Int J Mol Sci. 2020; 21(7):2556.
44. Agbani E O, Poole A W. Procoagulant platelets: generation, function, and therapeutic targeting in thrombosis. Blood. 2017; 130(20):2171-2179.
45. Tohidi-Esfahani I, Lee C S M, Liang H P H, Chen V M Y. Procoagulant platelets: Laboratory detection and clinical significance. Int J Lab Hematol. 2020; 42(Suppl1):59-67.
46. Pasalic L, Wing-Lun E, Lau J K, et al. Novel assay demonstrates that coronary artery disease patients have heightened procoagulant platelet response. J Thromb Haemost. 2018; 16(6):1198-1210.
47. Denorme F, Manne B K, Portier I, et al. Platelet necrosis mediates ischemic stroke outcome in mice. Blood. 2020; 135(6):429-440.
48. Prodan C I, Dale G L. Coated-platelets in ischemic stroke—potential insight into the etiology of stroke subtypes. International journal of stroke: official journal of the International Stroke Society. 2008; 3(4):249-250.
49. Kirkpatrick A C, Vincent A S, Dale G L, Prodan C I. Increased platelet procoagulant potential predicts recurrent stroke and TIA after lacunar infarction. J Thromb Haemost. 2020; 18(3):660-668.
50. Kirkpatrick A C, Stoner J A, Dale G L, Rabadi M, Prodan C I. Higher Coated-Platelet Levels in Acute Stroke are Associated with Lower Cognitive Scores at Three Months Post Infarction. J Stroke Cerebrovasc Dis. 2019; 28(9):2398-2406.
51. Batar P, Dale G L. Simultaneous engagement of thrombin and Fc gamma RIIA receptors results in platelets expressing high levels of procoagulant proteins. J Lab Clin Med. 2001; 138(6):393-402.
52. Fernández DI, Kuijpers M J E, Heemskerk J W M. Platelet calcium signaling by G-protein coupled and ITAM-linked receptors regulating anoctamin-6 and procoagulant activity. Platelets. 2020; 1-9.
53. Lo G K, Juhl D, Warkentin T E, Sigouin C S, Eichler P, Greinacher A. Evaluation of pretest clinical score (4 T's) for the diagnosis of heparin-induced thrombocytopenia in two clinical settings. J Thromb Haemost. 2006; 4(4):759-765.
54. Favaloro E J, McCaughan G, Mohammed S, et al. HIT or miss? A comprehensive contemporary investigation of laboratory tests for heparin induced thrombocytopenia. Pathology. 2018; 50(4):426-436.
55. Favaloro E J, Mohammed S, Donikian D, et al. A multicentre assessment of contemporary laboratory assays for heparin induced thrombocytopenia. Pathology. 2021; 53(2):247-256.
56. Worthington R E, Carroll R C, Boucheix C. Platelet activation by CD9 monoclonal antibodies is mediated by the Fc gamma II receptor. Br J Haematol. 1990; 74(2):216-222.
57. Morel-Kopp M-C, Mullier F, Gkalea V, et al. Heparin-induced multi-electrode aggregometry method for heparin-induced thrombocytopenia testing: communication from the SSC of the ISTH. J Thromb Haemost. 2016; 14(12):2548-2552.
58. Rauova L, Poncz M, McKenzie S E, et al. Ultralarge complexes of PF4 and heparin are central to the pathogenesis of heparin-induced thrombocytopenia. Blood. 2005; 105(1):131-138.
59. Tardy-Poncet B, Piot M, Chapelle C, et al. Thrombin generation and heparin-induced thrombocytopenia. J Thromb Haemost. 2009; 7(9):1474-1481.
60. Chilver-Stainer L, Lämmle B, Alberio L. Titre of anti-heparin/PF4-antibodies and extent of in vivo activation of the coagulation and fibrinolytic systems. Thromb Haemost. 2004; 91(2):276-282.
61. Cines D B, Yarovoi S V, Zaitsev S V, et al. Polyphosphate/platelet factor 4 complexes can mediate heparin-independent platelet activation in heparin-induced thrombocytopenia. Blood Adv. 2016; 1(1):62-74.

1. A method of diagnosing an immune-mediated or inflammatory prothrombotic condition in a subject, the method comprising:
a. Combining a sample of plasma obtained from the subject with a sample of whole blood obtained from a healthy donor to obtain a combined sample,
b. Contacting the combined sample with at least one agonist to obtain a test sample,
c. Determining the proportion of procoagulant platelets in the test sample and in at least one control sample, said determining comprising:
i. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
d. Determining whether the subject is suffering from the immune-mediated or inflammatory prothrombotic condition, wherein an increased proportion of procoagulant platelets in the test sample compared to the at least one control sample indicates that the subject is suffering from the immune-mediated or inflammatory prothrombotic condition.

2. The method of claim 1, wherein the assay control sample is obtained by contacting said test sample or said at least one control sample with GSCA and a control for the alpha granule detection agent.

3. The method of claim 1, wherein the increased proportion of procoagulant platelets in the test sample compared to the at least one control sample which indicates that the subject is suffering from the prothrombotic condition is an increase of at least about 1.1-fold.

4. The method of claim 1, wherein determining the uptake of GSAO and surface expression of alpha granule protein of platelets in step c.i. is carried out by flow cytometry.

5. The method of claim 1, wherein the healthy donor is a FcγRIIa high responder.

6. The method of claim 1, wherein the at least one agonist is selected from the group consisting of CRP-xL, SFLLRN, ADP, thrombin, TRAP, and combinations thereof.

7. The method of claim 1, wherein the immune-mediated or inflammatory prothrombotic condition is selected from the group consisting of heparin-induced thrombocytopenia (HIT), sepsis, anti-phospholipid syndrome (APS), vaccine-induced immune thrombotic thrombocytopenia (VITT), thrombotic thrombocytopenic purpura (TTP), atypical hemolytic uremic syndrome (HUS), systemic lupus erythematosus, thrombosis secondary to therapeutic monoclonal antibodies, viral pneumonia, human immunodeficiency viruses, dengue, and viral haemorrhagic fevers.

8. The method of claim 1, wherein the at least one control sample comprises a healthy control sample, which comprises healthy donor whole blood contacted with the agonist, and does not comprise subject plasma.

9. The method of claim 8, wherein the agonist is a combination of CRP-xL and thrombin, and optionally, wherein the increased proportion of procoagulant platelets in the test sample compared to the healthy control sample which indicates that the subject is suffering from the prothrombotic condition is an increase of at least about 1.1-fold.

10. (canceled)

11. The method of claim 1, wherein the immune-mediated or inflammatory prothrombotic condition is heparin-induced thrombocytopenia (HIT), and wherein step b. further comprises contacting the test sample with therapeutic concentration heparin, and wherein the at least one control sample comprises a no heparin control sample and a high heparin control sample, wherein the no heparin control sample comprises the combined sample, the agonist, and no heparin, and wherein the high heparin control sample comprises the combined sample, the agonist, and high concentration heparin.

12. (canceled)

13. The method of claim 11, wherein the therapeutic concentration of heparin is between about 0.1 and about 0.7 U/mL and wherein the high concentration of heparin is about 50 to 200 U/mL.

14. The method of claim 1, wherein the immune-mediated or inflammatory prothrombotic condition is heparin-induced thrombocytopenia (HIT) and wherein the at least one agonist is SFLLRN, and optionally wherein the proportion of procoagulant platelets in the test sample contacted with therapeutic concentration heparin compared to the no heparin control sample which indicates that the subject is suffering from HIT is an increase of between about 2 and 10-fold, and the proportion of procoagulant platelets in the test sample contacted with therapeutic concentration heparin compared to the high heparin control sample which indicates that the subject is suffering from HIT is an increase of between about 2 and 20-fold.

15. (canceled)

16. The method of claim 1, wherein the immune-mediated or inflammatory prothrombotic condition is vaccine-induced immune thrombotic thrombocytopenia (VITT) and the subject has been administered a vaccine.

17. The method of claim 16, wherein the vaccine is a recombinant adenoviral vector vaccine encoding the spike protein antigen of SARS-CoV-2.

18. (canceled)

19. The method of claim 16, wherein the at least one agonist is SFLLRN and wherein step b. further comprises contacting the test sample with therapeutic concentration heparin to obtain a therapeutic heparin sample, and further comprises contacting the test sample with high concentration heparin to obtain a high heparin sample, and
wherein step d. further comprises determining the proportion of procoagulant platelets in the therapeutic heparin sample and the high heparin sample, and
wherein the at least one control sample is a healthy control sample, comprising healthy donor whole blood contacted with the at least one agonist, and does not comprise subject plasma.

20. The method of claim 19, wherein the therapeutic concentration of heparin is between about 0.1 and about 0.7 U/mL and wherein the high concentration of heparin is about 50 to 200 U/mL.

21. The method of claim 19, wherein, compared to the healthy control sample:
(i) an increase in the proportion of procoagulant platelets in the test sample of greater than 2-fold, a change in the proportion of procoagulant platelets in the therapeutic heparin sample of up to 1.5-fold, and a change in the proportion of procoagulant platelets in the high heparin sample of up to 1.5-fold, indicates that the subject is suffering from classical VITT;
(ii) an increase in the proportion of procoagulant platelets in the test sample of greater than 1-fold, an increase in the proportion of procoagulant platelets in the therapeutic heparin sample of greater than 2-fold, and a change in the proportion of procoagulant platelets in the high heparin sample of up to 1.5-fold, indicates that the subject is suffering from heparin-enhancing VITT; and
(iii) a change in the proportion of procoagulant platelets in the test sample of up to 1.5-fold, a change in the proportion of procoagulant platelets in the therapeutic heparin sample of up to 1.5-fold, and a change in the proportion of procoagulant platelets in the high heparin sample of up to 1.5-fold, indicates that the subject is not suffering from VITT.

22. The method of claim 21, wherein the test sample is further contacted with the monoclonal antibody IV.3 and wherein, compared to the test sample:
(iv) a decrease in the proportion of procoagulant platelets in the test sample further contacted with the monoclonal antibody IV.3 to 0.7-fold or greater indicates that the subject is suffering from classical VITT or heparin-enhancing VITT.

23. The method of claim 16, wherein the at least one agonist is thrombin, wherein the at least one control sample is a healthy control sample, comprising healthy donor whole blood contacted with the at least one agonist, and does not comprise subject plasma, and optionally wherein the increased proportion of procoagulant platelets in the test sample compared to the healthy control sample which indicates that the subject is suffering from VITT is an increase of at least about 2-fold.

24. (canceled)

25. (canceled)

26. The method of claim 1, wherein the immune-mediated or inflammatory prothrombotic condition is antiphospholipid syndrome.

27. The method of claim 26, wherein the at least one control sample comprises a healthy control sample, which comprises healthy donor whole blood contacted with the agonist, and does not comprise subject plasma.

28. The method of claim 26, wherein the agonist is thrombin, and optionally wherein the increased proportion of procoagulant platelets in the test sample compared to the healthy control sample which indicates that the subject is suffering from the prothrombotic condition is an increase of at least about 1.1-fold.

29. (canceled)

30. A method of treatment of an immune-mediated or inflammatory prothrombotic condition in a subject or reducing side effects associated with a treatment of an immune-mediated or inflammatory prothrombotic condition in a subject, the method comprising:
a. Determining if the subject is suffering from the immune-mediated or inflammatory prothrombotic condition or is at risk of experiencing a thrombotic event, said determining comprising:
i. Combining a sample of plasma obtained from the subject with a sample of whole blood obtained from a healthy donor to obtain a combined sample,
ii. Contacting the combined sample with at least one agonist to obtain a test sample,
iii. Determining the proportion of procoagulant platelets in the test sample and in at least one control sample, said determining comprising:
1. Contacting the test sample and the at least one control sample with GSAO and an alpha granule detection agent, wherein platelets in the test sample and the at least one control sample which have both increased uptake of GSAO and increased surface expression of alpha granule protein compared to an assay control sample are identified as procoagulant platelets, and
iv. Determining whether the subject is suffering from the prothrombotic condition or is at risk of experiencing a thrombotic event, wherein an increased proportion of procoagulant platelets in the test sample compared to the at least one control sample indicates that the subject is suffering from the prothrombotic condition or is at risk of experiencing a thrombotic event,
b. Administering a treatment or escalating the treatment for the immune-mediated or inflammatory prothrombotic condition to the subject, only if the subject is determined to be suffering from the immune-mediated or inflammatory prothrombotic condition, or is at risk of experiencing a thrombotic event, respectively, and optionally,
c. Not administering or not escalating the treatment for the immune-mediated or inflammatory prothrombotic condition to the subject, if the subject is not at risk of experiencing a thrombotic event.

31. A method of selecting a therapy for treatment of an immune-mediated or inflammatory prothrombotic condition in a subject, the method comprising:
a. Determining if the therapy causes a reduction in the proportion of procoagulant platelets in the subject, said determining comprising:
i. Combining a sample of plasma obtained from the subject with a sample of whole blood obtained from a healthy donor to obtain a combined sample,
ii. Contacting the combined sample with at least one agonist to obtain a test sample,
iii. Contacting the test sample with the therapy to obtain a therapy test sample,
iii. Determining the proportion of procoagulant platelets in the test sample and in the therapy test sample, said determining comprising:
1. Contacting the test sample and the therapy test sample with GSAO and an alpha granule detection agent, wherein platelets in the test sample and the therapy test sample which have both increased uptake of GSAO and increased surface expression of alpha granule protein compared to an assay control sample are identified as procoagulant platelets,
b. Selecting the therapy for treatment of the immune-mediated or inflammatory prothrombotic condition if the proportion of procoagulant platelets in the therapy test sample is lower than the proportion of procoagulant platelets in the test sample.

32. (canceled)
